SEQUENCE LISTING (1 sequence)

SEQ ID NO: 1 - DNA, 56 bp, synthetic construct
  source 1..56: mol_type = other DNA; note = compaction oligonucleotide
  variation 54..56: note = 2'-O-methyl RNA bases may or may not be present
  misc_feature 1..53: note = DNA
  misc_feature 54..56: note = RNA
catgtaatgcacgtactttcagggtaaacatgtaatgcacgtactttcagggtnnn

(19) United States
(12) Patent Application Publication
(10) Pub. No.: US 2025/0349138 A1
(43) Pub. Date: Nov. 13, 2025

(54) THREE-DIMENSIONAL BASE CALLING IN NEXT GENERATION SEQUENCING ANALYSIS

(71) Applicant: Element Biosciences, Inc., San Diego, CA (US)

(72) Inventors: Connor THOMPSON, San Diego, CA (US); Tsung-li LIU, San Diego, CA (US); Semyon KRUGLYAK, San Diego, CA (US)

(21) Appl. No.: 19/170,863

(22) Filed: Apr. 4, 2025

Related U.S. Application Data
(63) Continuation of application No. PCT/US2023/076125, filed on Oct. 5, 2023.
(60) Provisional application No. 63/413,864, filed on Oct. 6, 2022.

(51) Int. Cl. G06V 20/69; G06T 5/20; G06T 5/50; G06T 5/70; G06T 7/30; G06V 10/60; G16H 30/40

(52) U.S. Cl. CPC G06V 20/695; G06T 5/20; G06T 5/50; G06T 5/70; G06T 7/30; G06V 10/60; G16H 30/40; G06T 2207/20224

(57) ABSTRACT

Disclosed herein are system, apparatus, method, and/or computer program product embodiments, and/or combinations and sub-combinations thereof which enables 3D base calling using flow cell images of samples such as in situ cells or tissue to ensure accurate base calling and sequencing analysis of 3D samples. Embodiments of the methods, systems, and media for 3D base calling of flow cell images includes image intensity, location, size, and/or of clusters or polonies to be relied on for accurate base calling.

### CROSS REFERENCE TO RELATED APPLICATIONS

This application is a continuation of PCT/US2023/076125 filed Oct. 5, 2023, which claims the benefit of U.S. Provisional Patent Application No. 63/413,864, filed Oct. 6, 2022, which is hereby incorporated by reference in its entirety.

### REFERENCE TO SEQUENCE LISTING SUBMITTED ELECTRONICALLY

The content of the electronically submitted Seqeunce Listing SML, File Name: 4585_0090002_SequenceListing_ST26, Size: 3.3 kilobytes, and Date of Creation: Apr. 4, 2025, submitted herewith, is hereby incorporated by reference in its entirety.

### TECHNICAL FIELD

This disclosure relates generally to base calling in DNA sequencing data analysis, and particularly to three-dimensional (3D) base calling.

### BACKGROUND

In next-generation sequencing (NGS) or NGS-like applications such as sequencing by synthesis, sequencing by binding, or sequencing by avidity, in order to identify the sequence of a target nucleic acid, a new strand is synthesized one nucleotide base at a time. During each sequencing cycle, one base attaches to any given strand. At the imaging step of each cycle, image(s) are recorded. A base-calling algorithm is applied to the image(s) to “read” the successive signals from each cluster or polony and convert the optical signals into an identification of the nucleotide base sequence added to each DNA fragment. Traditional base calling relies on two-dimensional (2D) flow cell images. When it comes to sequencing analysis of in situ samples such as cells or tissue, the sample has a thickness along the z direction orthogonal to the image plane. As such, flow cell images at a selected z level can include signals from out-of-focus polonies located at adjacent z levels and other undesired signals, e.g., from the cell membrane. There is a need for three-dimensional (3D) base calling to ensure accurate base calling and sequencing analysis of 3D samples such as cells and tissue.

### BRIEF SUMMARY

Provided herein are system, apparatus, method, and/or computer program product embodiments, and/or combinations and sub-combinations thereof which enables 3D base calling using flow cell images of samples such as in situ cells or tissue. The flow cell images can come from different sequencing cycles and/or different channels. The flow cell images may come from traditional two-dimensional samples or in situ samples. The flow cell image may come from sample of unbalanced nucleotide diversity.

As a particular application of such, embodiments of methods, systems, and media for 3D base calling of flow cell images, so that the image intensity, location, size, and/or of clusters or polonies can be relied on for accurate base calling.

Other embodiments of these aspects include corresponding computer systems, apparatus, and computer program product recorded on computer storage device(s), which, alone or in combination, configured to perform the actions of the methods. For a computer system configured or to be configured to perform operations or actions, the computer system has installed on it software, firmware, hardware, or their combinations that in operation cause the computer system to perform the operations or actions. For a computer program product configured or to be configured to perform operations or actions, the computer program product includes instructions that, when executed, by a hardware processor, cause the hardware processor to perform the operations or actions.

Further embodiments, features, and advantages of the present disclosure, as well as the structure and operation of the various embodiments of the present disclosure, are described in detail below with reference to the accompanying drawings.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the art(s) to make and use the embodiments.

FIG. 1 illustrates a block diagram of a system for performing 3D base calling of flow cell images, according to some embodiments.

FIGS. 2A-2C show exemplary flow cell images, processed images, and filtered images for 3D base calling, according to some embodiments.

FIGS. 3A-3C show exemplary flow cell images, processed images, and filtered images for 3D base calling, according to some embodiments.

FIGS. 3D-3E show an exemplary flow cell image and its corresponding filtered image for 3D base calling, according to some embodiments.

FIG. 4 illustrates a block diagram of a computer system for performing sequencing analysis and/or base calling, according to some embodiments.

FIG. 5 shows an exemplary projection image of flow cell images taken at different axial locations of a 3D sample, according to some embodiments.

FIG. 6A is a flow chart of an exemplary method of performing 3D base calling of flow cell images, according to some embodiments.

FIG. 6B is a flow chart of an exemplary method of performing 3D base calling of flow cell images, according to some embodiments.

FIG. 7A-7B show exemplary registration of a sequencing image of polonies with a cell staining image.

FIG. 8A shows a schematic diagram of flow cell images, subtiles, and regions of the flow cell images with polonies, according to some embodiments.

FIG. 8B shows a schematic diagram of a portion of a flow cell with multiple tiles, according to some embodiments.

FIG. 9 illustrates a flow chart of a method for performing image registration of flow cell images, according to some embodiments.

FIG. 10A-10B show a schematic diagram of an image transformation and corresponding 2D shifts, according to some embodiments.

FIG. 11 is a schematic showing of an exemplary linear single stranded library molecule (1100) which comprises: a surface pinning primer binding site (1120); an optional left unique identification sequence (1180); a left index sequence (1160); a forward sequencing primer binding site (1140); an insert region having a sequence of interest (1110); reverse sequencing primer binding site (1150); a right index sequence (1170); and a surface capture primer binding site (1130).

FIG. 12 is a schematic showing an exemplary linear single stranded library molecule (1100) which comprises: a surface pinning primer binding site (1120); a left index sequence (1160); a forward sequencing primer binding site (1140); an insert region having a sequence of interest (1110); a reverse sequencing primer binding site (1150); a right index sequence (1170); an optional right unique identification sequence (1190); and a surface capture primer binding site (1130).

FIG. 13 is a schematic showing exemplary embodiments of padlock probes.

FIG. 14 is a schematic showing a workflow for generating inside a cell circularized padlock probes, comprising generating first and second cDNAs from first and second target RNA molecules (respectively), hybridizing first and second padlock probes to the first and second cDNA molecules (respectively) to generate first and second circularized padlock probes (respectively). The first padlock probe comprises (i) a first target barcode sequence (target BC-1) that uniquely identifies the first target RNA or the first target cDNA, (ii) a first sequencing primer binding site (or a complementary sequence thereof), (iii) a universal binding site for an amplification primer (universal RCA) (or a complementary sequence thereof), and (iv) a universal binding site for a compaction oligonucleotide (or a complementary sequence thereof). The second padlock probe comprises (i) a second target barcode sequence (target BC-2) that uniquely identifies the second target RNA or the second target cDNA, (ii) a second sequencing primer binding site (or a complementary sequence thereof), (iii) a universal binding site for an amplification primer (universal RCA) (or a complementary sequence thereof), and (iv) a universal binding site for a compaction oligonucleotide (or a complementary sequence thereof).

FIG. 15 is a schematic showing a rolling circle and sequencing workflow inside a cell, comprising generating first and second concatemers by conducting rolling circle amplification using first and second covalently closed circular molecules (respectively). The first and second concatemers are subjected to a sequencing workflow using universal sequencing primers, sequencing polymerases, and a plurality of nucleotide reagents.

FIG. 16 is a schematic showing an exemplary workflow for sequencing a concatemer that is generated inside the cell. The concatemer includes tandem repeat units where each unit comprises: (i) a universal sequencing primer binding site (Seq), (ii) universal compaction oligonucleotide binding site (CO), (iii) an insert sequence that corresponds to a given target cDNA, and (iv) a target barcode sequence that corresponds to the given target cDNA (BC).

FIG. 17 is a schematic showing an exemplary workflow for sequencing a concatemer that is generated inside the cell. The concatemer includes tandem repeat units where each unit comprises: (i) a universal sequencing primer binding site (Seq), (ii) universal compaction oligonucleotide binding site (CO), (iii) an insert sequence that corresponds to a given target cDNA, and (iv) a target barcode sequence that corresponds to the given target cDNA (BC).

FIG. 18 is a schematic showing an exemplary workflow for sequencing a concatemer that is generated inside the cell. The concatemer includes tandem repeat units where each unit comprises: (i) a universal sequencing primer binding site (Seq), (ii) universal compaction oligonucleotide binding site (CO), and (iii) an insert sequence that corresponds to a given target cDNA.

FIG. 19 is a schematic showing an exemplary workflow for sequencing a concatemer that is generated inside the cell. The concatemer includes tandem repeat units where each unit comprises: (i) a universal sequencing primer binding site (Seq) and (ii) an insert sequence that corresponds to a given target cDNA.

FIG. 20 is a schematic showing a workflow for generating circularized padlock probes, comprising generating first and second cDNAs from first and second target RNA molecules (respectively), hybridizing first and second padlock probes to the first and second cDNA molecules (respectively) to generate first and second circularized padlock probes (respectively). The first padlock probe comprises (i) a first target barcode sequence (target BC-1) that uniquely identifies the first target RNA, (ii) a first batch-specific sequencing primer binding site (Batch Seq-1) (or a complementary sequence thereof), (iii) a universal binding site for an amplification primer (universal RCA) (or a complementary sequence thereof), and (iv) a universal binding site for a compaction oligonucleotide (or a complementary sequence thereof). The second padlock probe comprises (i) a second target barcode sequence (target BC-2) that uniquely identifies the second target RNA, (ii) a second batch-specific sequencing primer binding site (Batch Seq-2) (or a complementary sequence thereof), (iii) a universal binding site for an amplification primer (universal RCA) (or a complementary sequence thereof), and (iv) a universal binding site for a compaction oligonucleotide (or a complementary sequence thereof).

FIG. 21 is a schematic showing a rolling circle and sequencing workflow comprising generating first and second concatemers by conducting rolling circle amplification using first and second covalently closed circular molecules (respectively). The first and second concatemers are subjected to a first sequencing workflow using first batch-specific sequencing primers, sequencing polymerases, and a plurality of nucleotide reagents. The first concatemers undergo reiterative sequencing but the second concatemers do not. The first and second concatemers are subjected to a second sequencing workflow using second batch-specific sequencing primers, sequencing polymerases, and a plurality of nucleotide reagents. The second concatemers undergo reiterative sequencing but the first concatemers do not.

FIG. 22 is a schematic of an exemplary low binding support comprising a glass substrate and alternating layers of hydrophilic coatings which are covalently or non-covalently adhered to the glass, and which further comprises chemically-reactive functional groups that serve as attachment sites for oligonucleotide primers (e.g., capture oligonucleotides). In an alternative embodiment, the support can be made of any material such as glass, plastic or a polymer material.

FIG. 23 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 24 is a schematic of an exemplary multivalent molecule comprising a generic core attached to a plurality of nucleotide-arms.

FIG. 25 is a schematic of an exemplary multivalent molecule comprising a dendrimer core attached to a plurality of nucleotide-arms.

FIG. 26 shows a schematic of an exemplary multivalent molecule comprising a core attached to a plurality of nucleotide-arms, where the nucleotide arms comprise biotin, spacer, linker and a nucleotide unit.

FIG. 27 is a schematic of an exemplary nucleotide-arm comprising a core attachment moiety, spacer, linker and nucleotide unit.

FIG. 28 shows the chemical structure of an exemplary spacer (TOP), and the chemical structures of various exemplary linkers, including an 11-atom Linker, 16-atom Linker, 23-atom Linker and an N3 Linker (BOTTOM).

FIG. 29 shows the chemical structures of various exemplary linkers, including Linkers 1-9.

FIG. 30A shows the chemical structures of various exemplary linkers joined/attached to nucleotide units.

FIG. 30B shows the chemical structures of various exemplary linkers joined/attached to nucleotide units.

FIG. 30C shows the chemical structures of various exemplary linkers joined/attached to nucleotide units.

FIG. 31 shows the chemical structure of an exemplary biotinylated nucleotide-arm. In this example, the nucleotide unit is connected to the linker via a propargyl amine attachment at the 5 position of a pyrimidine base or the 7 position of a purine base.

FIG. 32 is a schematic of a guanine tetrad (e.g., G-tetrad).

FIG. 33 is a schematic of an exemplary intramolecular G-quadruplex structure.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, method, and/or computer program product embodiments, and/or combinations and sub-combinations thereof which enables base calling using flow cell images acquired from three-dimensional (3D) samples, such as in situ cells or tissues. The 3D base calling techniques herein can be used on flow cell images obtained from various imaging and/or sequencing techniques. The techniques disclosed herein are useful for base calling in next generation sequencing, and base-calling will be used as the primary example herein for describing the application of these techniques. However, such image analysis techniques may also be useful in other applications where spot-detection and/or CCD imaging is used.

With traditional DNA sequencing, the optical system can be tuned to be in-focus on the clusters or polonies of two dimensional (2D) samples. The flow cell images may show clusters or polonies as bright spots in 2D. Base callings can be performed using corresponding image intensities of the bright spots. However, in situ samples such as cells or tissue can have a thickness along the axial axis, i.e., z direction, that cannot be in-focus within a single 2D image. Thus, a stack of multiple 2D flow cell images at different axial locations may be acquired to cover clusters or polonies of the in situ samples. Interferences may occur in the stack of flow cell images, such as from out-of-focus polonies and background signals of cellular components like cell membrane. For example, a polony that locates at a first axial location can appear in a first flow cell image and it may also generate a blob of signal in a second 2D flow cell image taken at its adjacent axial location where it is out-of-focus. The blob of signal may interfere with intensities of polonies at or near the same x-y location in the second flow cell image, thus deteriorating the accuracy and reliability of base callings. The techniques disclosed herein can be configured for processing the stack of flow cell images of a 3D sample and generating accurate and reliable image intensities for polonies or clusters, thus accurate and reliable base callings of 3D samples. Existing algorithms for processing image intensity from a volumetric 3D sample can suffer from various shortcomings. For example, flattening the stack of images without removing signal interferences from large background components like membrane or cytosol may result in unreliable image intensities and inaccuracy in base calling. Additionally, the out-of-focus polonies or clusters may remain after certain 3D to 2D flattening methods and contribute to erroneous base calling. In some embodiments, flattening the stack of 2D flow cell images into a single 2D image, e.g., via projection, may cause loss of polonies or clusters that are in-focus but whose intensities blend into the out-of-focus polonies. Further, existing sequencing and analysis methods for 3D samples may fail to provide sufficient resolution along z axis and may fail to enabling sequencing and analysis when the 3D sample is of high density (e.g., 2×, 4×, 5×, or more than what the traditional sequencing method can possibly handle with a predetermined quality, e.g., Q30, Q35, or Q40) and/or unbalanced nucleotide diversity. Thus, there is a need for generating accurate and reliable image intensities for polonies or clusters from 3D volumetric samples so that such image intensities can be used for accurate and reliable 3D base callings.

In some embodiments, the techniques disclosed herein advantageously filters the flow cell images before flattening the axial stack of 2D images to a single 2D image so that the out-of-focus polonies or clusters can be removed efficiently without effecting in-focus polonies or clusters. The flattening of the stack of flow cell images herein advantageous finds the intensity of each polony or cluster where its in-focus. The techniques disclosed herein advantageously utilize images that retain background information for accurate and efficient registration of the polonies or clusters relative to cellular components, e.g., nucleus, in the cells.

In some embodiments, the techniques disclosed herein advantageously generate a 3D polony map. The techniques herein may efficiently filter out-of-focus polonies and background objects without effecting in-focus polonies or clusters in the 3D polony map. The 3D polony map can be used for extracting polony intensities for base callings. Comparing with a single flattened 2D image, the 3D polony map advantageously retains information of polonies and clusters that may be removed in the flattened image. The 3D polony map can be generated in a few early flow cycles and used in subsequent flow cycles so that the additional computational load of recalculating new polony maps and the storage space for saving them can be minimized. The techniques disclosed herein advantageously utilize images that retain background information for accurate and efficient registration of the polonies or clusters to cells and such background information may facilitate sequencing analysis by providing spatial information of the polonies or clusters relative to the cellular components. Further, the techniques disclosed herein advantageously remove duplicate polonies and decompose polonies that may partially overlap with each other that may cause errors for accurate and reliable base calling in 3D

In DNA sequencing, identifying the centers of clusters or polonies is sometimes referred to as part of primary analysis. Primary analysis can include some or all of operations and/or steps needed to perform base calling and compute quality score of the base callings. Primary analysis can involve the formation of a template image for at least part of the flow cell. The template image can include the estimated locations of all detected clusters or polonies in a common coordinate system. Template images are generated by identifying cluster or polony locations in all images in the first few cycles of the sequencing process.

### Sequencing Systems

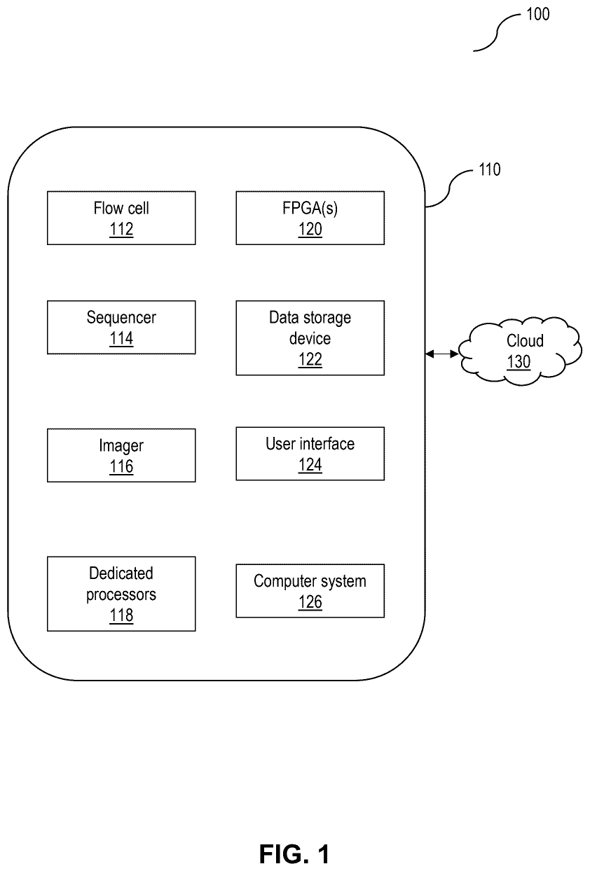

In some embodiments, sequencing and sequencing analysis of samples are performed using a computer implemented system here. FIG. 1 illustrates a block diagram of a computer-implemented system 100, according to one or more embodiments disclosed herein. The system 100 has a sequencing system 110 that includes a flow cell 112, a sequencer 114, an imager 116, data storage 122, and user interface 124. The sequencing system 110 may be connected to a cloud 130. The sequencing system 110 may include one or more of dedicated processors 118, Field-Programmable Gate Array(s) (FPGAs) 120, and a computer system 126.

In some embodiments, the flow cell 112 is configured to capture DNA fragments and form DNA sequences for base-calling on the flow cell. The flow cell 112 can include a support as disclosed herein. The support can be a solid support. The support can include a surface coating thereon as disclosed herein. The surface coating can be a polymer coating as disclosed herein.

A flow cell 112 can include multiple tiles or imaging areas thereon, and each tile may be separated into a grid of subtiles. Each subtile can include a plurality of clusters or polonies thereon. As a nonlimiting example, a flow cell can have 424 tiles, and each tile can be divided into a 6×9 grid, therefore 54 subtiles. The flow cell image as disclosed herein can be an image including signals of a plurality of clusters or polonies. The flow cell image can include one or more tiles of signals or one or more subtiles of signals. In some embodiments, a flow cell image can be an image that includes all the tiles and approximately all signals thereon. The flow cell image can be acquired from a channel during an imaging or sequencing cycle using the imager 116. In some embodiments, each tile may include millions of polonies or clusters. As a nonlimiting example, a tile can include about 1 to 10 million of clusters or polonies. Each polony can be a collection of many copies of DNA fragments.

In embodiments where three-dimensional (3D) samples, e.g., cells or tissues are immobilized on the flow cell, are sequenced, the flow cell images may be acquired at multiple z levels which are orthogonal to the image plane of the flow cell images to cover the volume of the 3D sample. The z axis can extend from the objective lens of the optical system disclosed herein to the support, e.g., flow cell device. Each z level of flow cell images may be parallel to and separated from the adjacent z level(s) for a predetermined distance, for example, for about 0.1 um to about 15 ums. Each z level may include a predetermined thickness. The thickness may be in the range from 0.01 um to 5 um. In some embodiments, the thickness may be determined so that a pixel has isotropic size in x, y, and z direction. In other words, the pixel or voxel is a cube. Each flow cell image may include a thickness, e.g., in-focus depth, of 0.01 um to 0.9 um. In some embodiments, each flow cell image may include a thickness in the range from 0.05 um to 0.5 um. In some embodiments, each flow cell image may include a thickness in the range from 0.1 um to 0.3 um.

Flow cell images at each z-level may be separated from the adjacent level(s) for 0.01 um to 10 ums, e.g., between the centers of flow cell images at the adjacent levels. Each z level of flow cell images at its center may be separated from the center of the adjacent level(s) for 0.1 um to 5 ums. In some embodiments, a number of z levels is predetermined to allow coverage of some or all of the 3D volume of the sample expanding along the z axis. For example, for a sample of 10 um thickness, 10, 11, 12, or more z levels that are about 1 um thick and 1 um apart from each other may be used to cover the sample along z axis without overlapped coverage along z axis. There may be no gap in between the thicknesses of flow cell images at adjacent z levels. As another example, for a sample of 10 ums thickness, 20, 21, or 22 z levels that are 0.5 um thick and 0.6 um apart from each other may be used to cover the sample along z axis with a gap of 0.1 um between flow cell images at adjacent z levels.

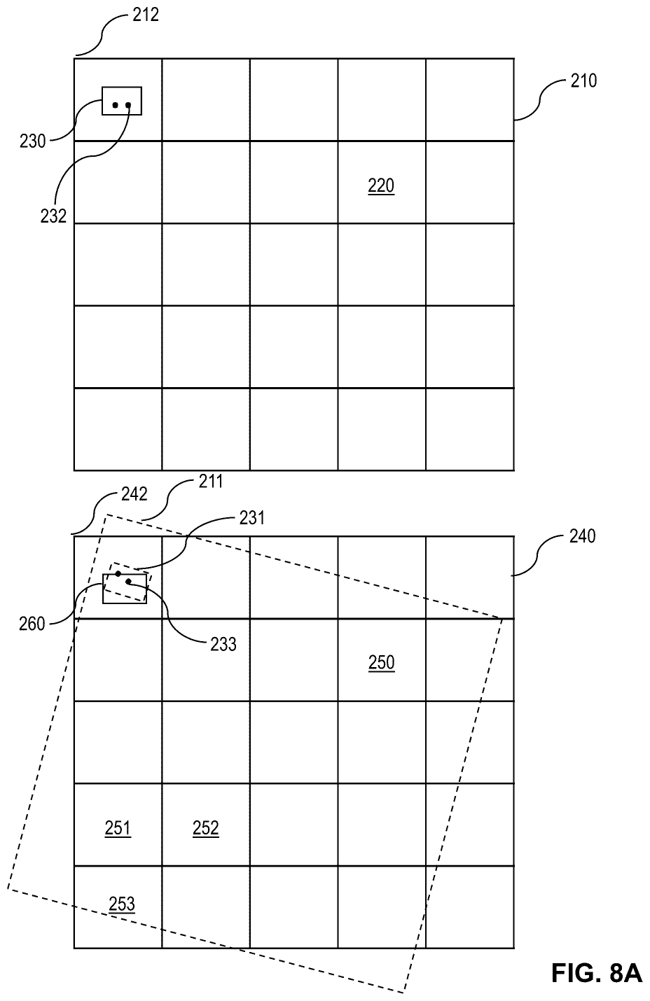

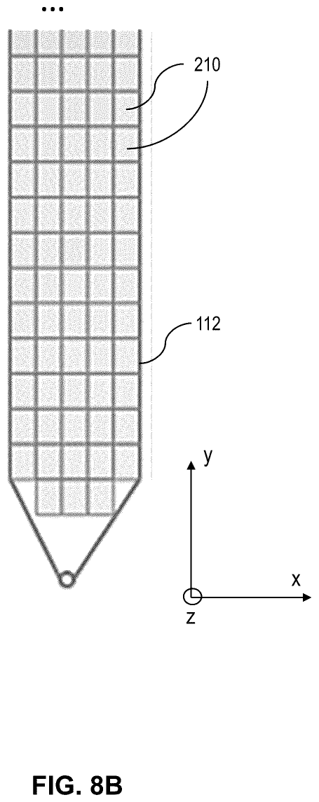

At each z-level, flow cell image(s) can be acquired from one or more sequencing cycles and/or one or more channels. Each flow cell image may include in its field of view at least part of one or more tiles or subtiles of the flow cell. FIG. 8B shows a portion of a flow cell 112 with multiple tiles 210. The image plane is defined by the x and y axis. And the z axis is orthogonal to the x-y plane. Although the flow cell images, samples, and the z axis are described in a Cartesian coordinate system, any other coordinate systems can be used to define spatial locations and relationships of the polonies or clusters and their images herein. Other coordinate systems can include but are not limited to the polar coordinate system, cylindrical, or spherical coordinate systems.

The sequencer 114 may be configured to flow a nucleotide mixture onto the flow cell 112, cleave blockers from the nucleotides in between flowing steps, and perform other steps for the formation of the DNA sequences on the flow cell 112. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths. In some embodiments, the sequencer 114 and the flow cell 112 may be configured to performing various sequencing methods disclosed herein, for example, sequencing-by-avidite.

For example, each nucleotide base may be assigned a color. Different types of nucleotides can have different colors. Adenine (A) may be red, cytosine (C) may be blue, guanine (G) may be green, and thymine (T) may be yellow, for example. The color or wavelength of the fluorescent element for each nucleotide may be selected so that the nucleotides are distinguishable from one another based on the wavelengths of light emitted by the fluorescent elements.

The imager 116 may be configured to capture images of the flow cell 112 after each flowing step. In an embodiment, the imager 116 is a camera configured to capture digital images, such as a CMOS or a CCD camera. The camera may be configured to capture images at the wavelengths of the fluorescent elements bound to the nucleotides. The images can be called flow cell images.

In some embodiments, the imager 116 can include one or more optical systems disclose herein. The optical system(s) can be configured to capture optical signals from the flow cell and generate corresponding digital images thereof. The digital images can then be used for base calling.

In an embodiment, the images of the flow cell may be captured in groups, where each image in the group is taken at a wavelength or in a spectrum that matches or includes only one of the fluorescent elements. In another embodiment, the images may be captured as single images that captures all of the wavelengths of the fluorescent elements.

The resolution of the imager 116 can control the level of detail in the flow cell images, including pixel size. In existing systems, this resolution is very important, as it controls the accuracy with which a spot-finding algorithm identifies the polony centers. In some embodiments, the image resolution of flow cell images disclosed herein can be about 10 nanometers (nms) to a couple of hundreds of nms or greater. In some embodiments, the image resolution of flow cell images disclosed herein can be about 10 nanometers (nms) to a couple of microns or greater. One way to increase the accuracy of spot finding is to improve the resolution of the imager 116, or improve the processing performed on images taken by imager 116. Detecting polony centers in pixels other than those detected by a spot-finding algorithm can be performed. These methods can allow for improved accuracy in detection of polony centers without increasing the resolution of the imager 116. The resolution of the imager may even be less than existing systems with comparable performance, which may reduce the cost of the sequencing system 110.

The image quality of the flow cell images can control the base calling quality. One way to increase the accuracy of base calling is to improve the imager 116, or improve the processing performed on images taken by imager 116 to result in a better image quality.

The methods described herein are configured to register the flow cell images to a common coordinate system so that the base calling with respect to a cluster or polony can be more accurate than without such registration. These methods can allow for accurate and efficient base calling.

The methods herein can be advantageously performed in parallel in the computer-implemented system 100, without interference with or delay of existing sequencing workflow of the system 100. After flow cell images are acquired in a particular cycle, image registration and other processing of such flow cell images can be performed while sequencing of the currently cycle or the subsequent cycle(s) is in progress. Such image processing and base calling operations performed in parallel may advantageously speed up the sequencing analysis process and reduce total time of sequencing and corresponding time. Base calling may also be performed while sequencing of the current cycle or the subsequent cycle(s) is in progress. Further, some or all of the operations disclosed herein can be advantageously performed by the FPGA(s) and/or NPU(s) and data can be communicated between the CPU(s) and FPGA(s) and/or NPU(s) to reduce the total operational time from methods operating without the FPGA(s).

The methods herein can be advantageously performed with less storage space needed than traditional sequencing analysis methods where the flow cell images are stored. The image processing and base calling in parallel with sequencing reactions may advantageously allow storage of the flow cell images only until base calling is performed in parallel thereby eliminating the need to store flow cell images until the end of the sequencing run and free-up storage space of the system. Instead of directly storing multiple flow cell images before and/or after image processing, e.g., image registration, image intensities, and corresponding locations of selected polonies are saved for base calling. Thus, the methods disclosed here are computationally less intensive than traditional methods so that the heat dissipation by the computer/processors can be easier to manage and less likely to cause undesired disturbance to the chemistry of sequencing reactions disclosed herein. In addition, transformation matrixes instead of flow cell images can be saved, which can save memory space needed and improve efficiency of the operations for performing 3D base calling.

The sequencing system 110 may be configured to perform image processing of the flow cell images across different cycles and/or channels. The operations or actions disclosed herein may be performed by the dedicated processors 118, the FPGA(s) 120, the computing system 126, or a combination thereof. One or more operations or actions in methods 600 disclosed herein may be performed by the dedicated processors 118, the FPGA(s) 120, the computing system 126, or a combination thereof. In some embodiments, which operations or actions are to be performed by performed by the dedicated processors 118, the FPGA(s) 120, the computing system 126, or their combinations can be determined based on one or more of: a computation time for the specific operation(s), the complexity of computation in the specific operation(s), the need for data transmission between the hardware devices, or their combinations. Image processing such as image registration disclosed herein can be performed after the flow cell images are acquired but before base calling of the flow cell images is performed in a cycle.

The computing system 126 can include one or more general purpose computers that provide interfaces to run a variety of program in an operating system, such as Windows™ or Linux™ Such an operating system typically provides great flexibility to a user.

In some embodiments, the dedicated processors 118 may be configured to perform operations in the methods disclosed herein. They may not be general-purpose processors, but instead custom processors with specific hardware or instructions for performing those steps. Dedicated processors directly run specific software without an operating system. The lack of an operating system reduces overhead, at the cost of the flexibility in what the processor may perform. A dedicated processor may make use of a custom programming language, which may be designed to operate more efficiently than the software run on general-purpose computers. This may increase the speed at which the steps are performed and allow for real time processing.

In some embodiments, the dedicated processors 1180 or the computing system 1260 may comprise reconfigurable logic devices, such as artificial intelligence (AI) chips, neural processing units (NPUs), application specific integrated circuits (ASICs), or a combination there of. The reconfigurable logic devices may be configured to perform one or more operations herein. The reconfigurable logic devices may be configured to perform one or more operations herein and accelerate the operations by allowing parallel data processing in comparison to CPUs.

In some embodiments, the FPGA(s) 120 may be configured to perform some or all of operations in the methods herein. An FPGA is programmed as hardware that will only perform a specific task. A special programming language may be used to transform software steps into hardware componentry. Once an FPGA is programmed, the hardware directly processes digital data that is provided to it without running software. The FPGA instead may use logic gates and registers to process the digital data. Because there is no overhead required for an operating system, an FPGA generally processes data faster than a general-purpose computer. Similar to dedicated processors, this is at the cost of flexibility.

The lack of software overhead may also allow an FPGA to operate faster than a dedicated processor, although this will depend on the exact processing to be performed and the specific FPGA and dedicated processor.

A group of FPGA(s) 120 may be configured to perform the steps in parallel. For example, a number of FPGA(s) 120 may be configured to perform a processing step for an image, a set of images, a subtile, or a select region in one or more images. Each FPGA(s) 120 may perform its own part of the processing step at the same time, reducing the time needed to process data. This may allow the processing steps to be completed in real time. Further discussion of the use of FPGAs is provided below.

Performing the processing steps in real time may allow the system to use less memory, as the data may be processed as it is received. This improves over conventional systems may need to store the data before it may be processed, which may require more memory or accessing a computer system located in the cloud 130.

In some embodiments, the data storage 122 is used to store information used in the methods herein. This information may include the flow cell images themselves or information and/or images derived from the flow images captured by the imager 116. The DNA sequences determined from the base-calling may be stored in the data storage 122. Parameters identifying polony locations may also be stored in the data storage 122. Raw and/or processed image intensities of each polony may be stored in the data storage. The region and/or subtile that each polony corresponds to may also be stored in the data storage 122. The transformation matrix of each region and/or subtile for different cycle(s) and/or channel(s) may also be stored in the data storage 122. Cell images may be stored in the data storage. The flow cell images, the processed images, and/or the filtered images may be stored in the data storage. Other information or images that can facilitate 3D base calling of the sample can be saved in the data storage.

The user interface 124 may be used by a user to operate the sequencing system or access data stored in the data storage 122 or the computer system 126.

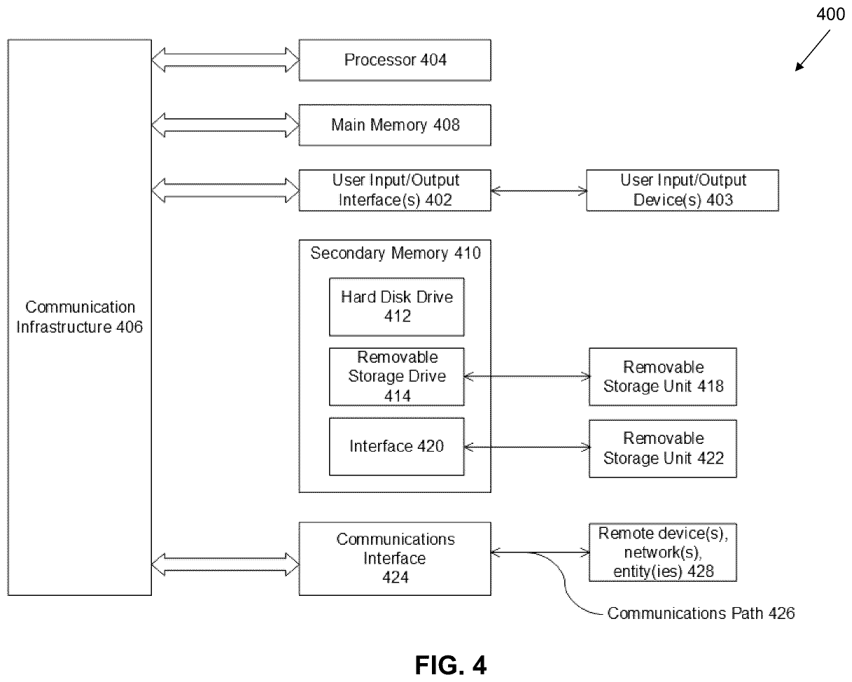

The computer system 126 may control the general operation of the sequencing system and may be coupled to the user interface 124. It may also perform steps in image processing, base calling, their preceding operations, and/or subsequent operations including but not limited to image registration. In some embodiments, the computer system 126 is a computer system 400, as described in more detail in FIG. 4. The computer system 126 may store information regarding the operation of the sequencing system 110, such as configuration information, instructions for operating the sequencing system 110, or user information. The computer system 126 may be configured to pass information between the sequencing system 110 and the cloud 130.

As discussed above, the sequencing system 110 may have dedicated processors 118, FPGA(s) 120, or the computer system 126. The sequencing system may use one, two, or all of these elements to accomplish necessary processing described above. In some embodiments, when these elements are present together, the processing tasks are split between them. For example, the FPGA(s) 120 may be used to perform some or all of: the preprocessing operations, image processing, image registration, base calling, and any subsequent operations, while the computer system 126 may perform other processing functions for the sequencing system 110 such as registering images for base calling with cell staining image(s). Those skilled in the art will understand that various combinations of these elements will allow various system embodiments that balance efficiency and speed of processing with cost of processing elements.

The cloud 130 may be a network, remote storage, or some other remote computing system separate from the sequencing system 110. The connection to cloud 130 may allow access to data stored externally to the sequencing system 110 or allow for updating of software in the sequencing system 110.

### 3D Base Callings Based on Flattened 2D Images

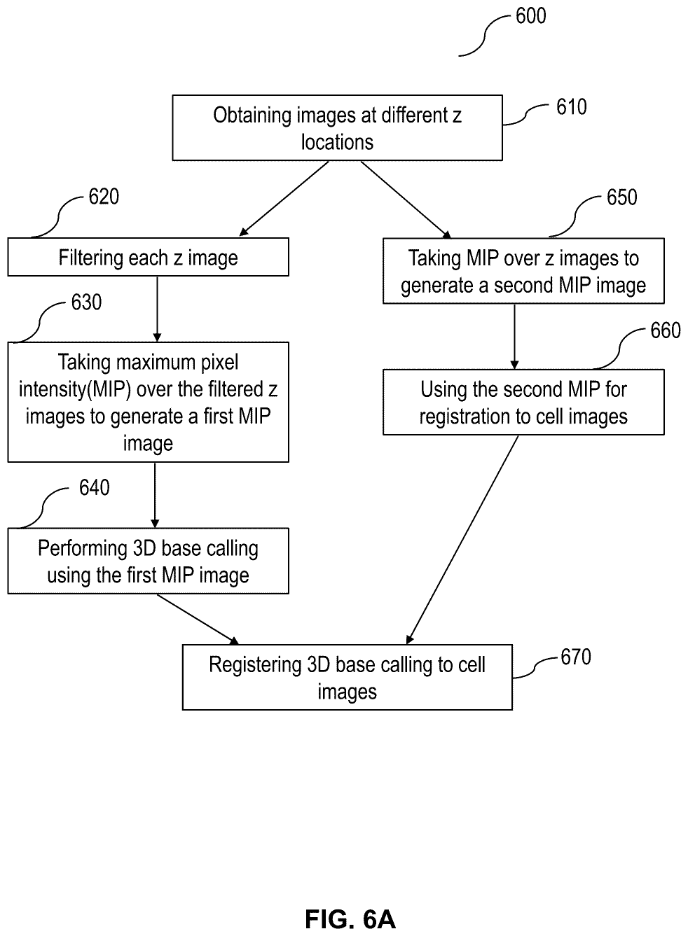

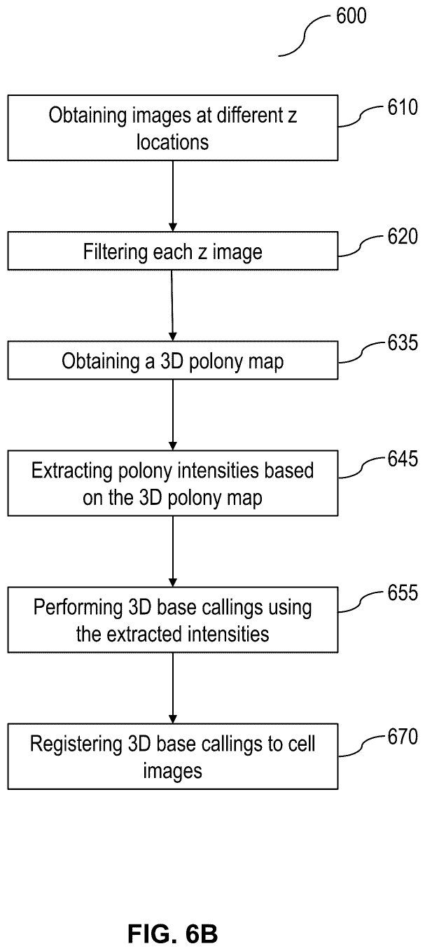

FIG. 6A shows a flow chart of an exemplary embodiment of the computer-implemented method 600 for performing 3D base calling based on the flow cell images. The method 600 can include some or all of the operations disclosed herein. The operations may be performed in but is not limited to the order that is described herein.

The method 600 can be performed by one or more processors disclosed herein. In some embodiments, the processor can include one or more of: a processing unit, an integrated circuit, or their combinations. For example, the processing unit can include a central processing unit (CPU, an artificial intelligence (AI) chip, a neural processing unit (NPU), and/or a graphic processing unit (GPU)). The integrated circuit can include a chip such as a field-programmable gate array (FPGA). In some embodiments, the processor can include the computing system 400. In some embodiments, some of the operations in method 600 can be performed by FPGA(s) and some other operations in method 600 are performed by AI chips or NPUs to improve energy consumption, heat dissipation, and/or computational time needed for sequencing analysis.

In some embodiments, some or all operations in method 600 can be performed by the FPGA(s). In embodiments when some operations are performed by FPGA(s), the data after an operation performed by the FPGA(s) can be communicated by the FPGA(s) to the CPU(s) so that CPU(s) can perform subsequent operation(s) in method 600 using such data. Similarly, data can also be communicated from the CPU(s) to the FPGA(s) for processing by the FPGA(s). In some embodiments, all the operations in method 600 can be performed by CPU(s). Alternatively, the operations performed by CPU(s) can be performed by other processors such as the dedicated processors, or NPU(s). In some embodiments, all the operations in method 600 can be performed by FPGA(s) and/or NPU(s).

The method 600 can comprise an operation 610 of obtaining multiple flow cell images of one or more samples. The flow cell images can be acquired at different z levels along an axial axis, i.e., the z axis. In some embodiments, the operation 610 comprises actively retrieving or passively receiving multiple flow cell images of a sample to be processed. In some embodiments, the operation 610 comprises acquiring the flow cell images using the imager 116 of the sequencing system.

In some embodiments, the operation 610 comprises: detecting and the fluorescent signal and color emitted by polonies and clusters by one or more image sensors of the optical system. The one or more sensors correspond to 2, 3, 4, or more color channels of the sequencing system. In some embodiments, a single image sensor may correspond to a single channel or more than one color channel of the sequencing system.

The sample can be in situ. The sample can be a 3D sample. The sample can be a volumetric sample that may contain different biological information at the same x-y location but different z location. The sample can include multiple cells, tissue, or their combination. The 3D sample can be any biological sample that has a thickness that is greater than a predetermined threshold along the axial axis. For example, the thickness can be greater than 2 um, 3 um, 4 um, 5 um, 10 um, 20 um, or more. The z axis (e.g., axial axis) is orthogonal to the image plane defined by x and y axes, as show in FIG. 8B.

The plurality of flow cell images herein may be acquired using the optical system disclosed herein, from 1, 2, 3, 4, or more channels of the imager 116. In some embodiments, the plurality of flow cell images are acquired in a single flow cycle or multiple flow cycles of a sequence run. Each flow cell image can include one or more tiles 210 (imaging areas), and each tile can be divided into multiple subtiles. Each subtile can include a plurality of polonies or clusters. Each subtile can include multiple regions with each region including a number of polonies. For example, the polonies can be extracted or otherwise identified from corresponding regions of flow cell images from 4 different channels in a cycle. As another example, the polonies can be extracted from flow cell images from a single channel. The flow cell image as disclosed herein can be images that are acquired from imaging sample(s) immobilized on the flow cell 112 as shown in FIG. 8B.

The flow cell 112 may include sample(s) immobilized thereon. The sample(s) may include a plurality of nucleic acid template molecules. The sample(s) may include a three-dimensional (3D) volumetric sample(s) or two dimensional (2D) sample(s). The nucleic acid template molecules may be distributed randomly or in various patterns on the flow cell 112. In some embodiments, the plurality of polonies or clusters herein may be extracted from specific regions of a tile, e.g., each subtile. With each subtile, the polonies may be extracted with a predetermined pattern or randomly.

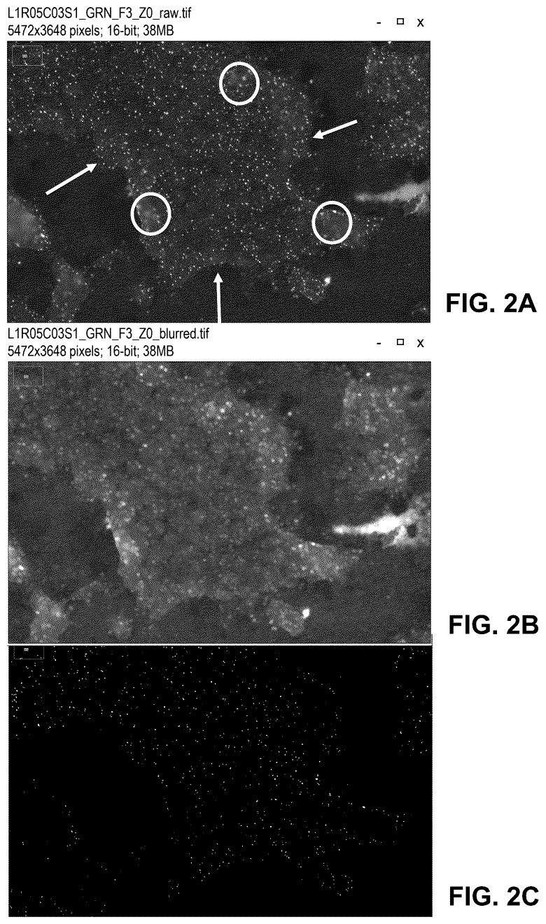

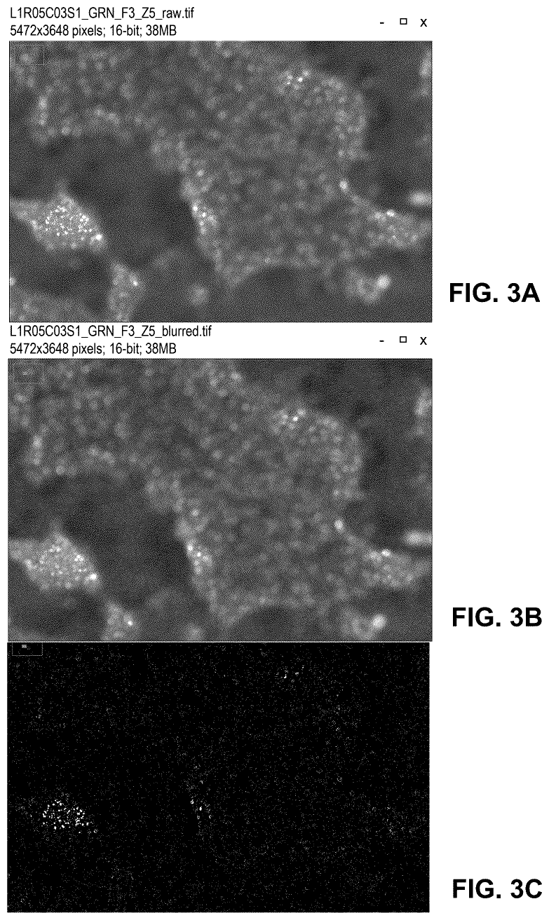

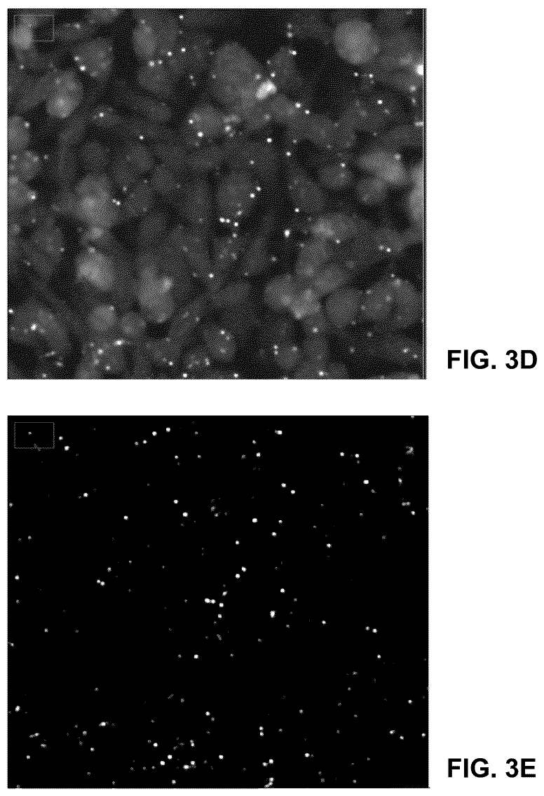

In some embodiments, a flow cell image herein can be an image of one or more tiles, one or more subtiles, one or more segmented regions within tile(s) or subtile(s), or their combinations. Each flow cell image can comprise a field of view (FOV). The FOV can be orthogonal to the axial axis. The FOV can be within the x-y plane. The FOV of different flow cell images at different axial locations can be identical within the x-y plane. The FOV of different flow cell images at different axial locations can have at least an overlapping portion within the x-y plane. The image resolution of different flow cell images at different axial locations can be about identical or exactly identical. In some embodiments, the image resolution of different flow cell images at different axial locations is different. FIGS. 2A and 3A show two exemplary flow cell images acquired at two different z levels along the axial axis of a same 3D sample within a same sequencing cycle. In some embodiments, the image resolution of the flow cell images along x, y, and/or z axis may be in a range from 0.001 um to 5 um. In some embodiments, the image resolution of the flow cell images along x, y, and/or z axis may be in a range from 0.01 um to 2 um. In some embodiments, the image resolution of the flow cell images along x, y, and/or z axis may be in a range from 0.02 um to 1 um.

Each flow cell image at a specific z level includes intensities generated by polonies and clusters at the corresponding z location. As shown in FIGS. 2A-3A, signals from polonies and clusters are small bright spots within the images. Each bright spot can be of various sizes that is less than a couple of pixels, e.g., less than a pixel, about a pixel, about 2 pixels, 3 pixels, 4, pixels, or 5 pixels. In some embodiments, each signal spot of the polonies or clusters can be any number of pixels in the range from 0.01 pixel to about 72 pixels. In some embodiments, each signal spot of the polonies or clusters can be any number of pixels in the range from 0.1 pixel to about 16 pixels.

Each flow cell image can also include intensities generated by the cell and its structural elements. Such structural elements can be background objects or components.

In some embodiments, when the depth of field the optical system includes a range, e.g., 0.1 um, 0.2 um, 0.3 um, 0.5 um, 0.6 um, 0.8 um, 1 um, 2 um, 3, um, 4 um, 5 um, etc. expanding along z axis. Polonies and clusters that are within the range of depth of field can appear in-focus or about in-focus in the flow cell image. Flow cell images at a specific z level can also include signals from polonies and clusters that are not within the focus range of the image. Such polonies or clusters are out-of-focus. As shown in FIG. 3A, bigger and blurry signal spots represent out-of-focus polonies or clusters. Some of the out-of-focus polonies or clusters are circled in FIG. 3A.

Each flow cell image at a specific z level can also include noises caused by the optical system and/or undesired signal from the sample. The undesired signal can be signal coming from components of the sample such as membrane, cytosol, and mitochondria. Such background objects can be any objects, relatively larger in size than the polonies or clusters. As shown in FIG. 3A, there is a blurry cellular contour (at the arrows) in the flow cell image, and most of the signal spots are contained within the blurry contour. FIG. 3D shows multiple cells and the polonies or clusters as small bright spots are generally within the contours of different cells. In some embodiments, background objects can include any objects within the 3D sample but are not polonies or clusters.

In some embodiments, the polonies or clusters being sequenced in a flow cycle may have a certain nucleotide diversity, e.g., in base calling. The method 600 may allow 3D base calling of flow cell images even if the polonies or clusters are of low or unbalanced diversity in sequencing cycle(s) with a predetermined base calling quality level. The nucleotide diversity of a population of nucleotide acid molecules, e.g., polonies or clusters, can refer to the relative proportion of nucleotides A, G, C, and T/U that are present in each flow cycle. The relative proportion of nucleotides may be within a region of the field of view or within the entire flow cell image. An optimally high or balanced diversity data can generally have approximately equal proportions of all four nucleotides represented in each flow cycle of a sequencing run. A low or unbalanced diversity data can generally include a high proportion of certain nucleotides and low proportion of other nucleotides in some flow cycles of a sequencing run, e.g., less than 10% of the total number of all 4 nucleotides. As a result, images corresponding to the high portion of certain nucleotides can have more signal spots (polonies or clusters) than images corresponding to the low portion of certain nucleotides. As an example of low or unbalanced diversity data, the bases A, T, C, G can be about 1%, about 2%, about 1%, and about 95%, respectively, of the total number of polonies, in a certain flow cycle. Subsequently, the flow cell images from channels corresponding to A, T, and C in this particular flow cycle are darker and/or with much fewer polonies or clusters than the flow cell image corresponding to nucleotide G. As another example of low or unbalanced diversity data, the bases A, T, C, G in polonies in multiple flow cycles can be about 2%, about 5%, about 10%, and about 83%, respectively. In embodiments where low or unbalanced diversity data is present in a particular cycle and is imaged for sequencing analysis, image registration and subsequent base calling using existing technologies may fail because image(s) from one or more channels are too dark (e.g., signal spots of polonies are too sparse and/or dim) comparing with images acquired from other channels thereby causing problems in sequencing analysis. Further, in embodiments where low or unbalanced diversity data is present in a particular cycle, existing methods may fail to identify polonies or clusters that are dimer or sparser from other background information, e.g., cellular structural. The predetermined quality level can be customized based on different sequencing applications. For example, the predetermined quality level may be at least Q 30, Q35, Q40 or higher. As another example, the predetermined quality level may be less than 1%, 0.5%, 0.1%, 0.05%, 0.01%, 0.005%, 0.001% or less errors in the base callings. In some embodiments, the methods 600 are configured to perform base calling of 3D samples even if the polonies or clusters are of low diversity in some regions of the flow cell during some flow cycles from the 3D sample.

In some embodiments, the methods 600 are configured to process flow cell images, e.g., including intensity processing, registration, and base calling, even if the polonies or clusters are of unbalanced nucleotide diversity in one or more flow cycles with a predetermined quality level.

In some embodiments, the methods 600 are configured to process flow cell images, e.g., including intensity processing, registration, and base calling, even if the polonies or clusters are of higher spatial density than what can be handled using existing 3D sequencing methods. In some embodiments, the 3D sample may have a spatial density that is 2×, 4×, 6×, 8×, 10×, 12×15×, or more than the maximal spatial density manageable by existing 3D sequencing systems with a predetermined quality in base calling, e.g., Q40 or Q30. In some embodiments, the 3D sample may have a spatial density that is greater than about 0.01 to about 0.5 polonies/um³. As another example, the 3D sample can have a spatial density that is no less than 0.01 to 0.5 polonies/um³. In some embodiments, the 3D sample can have a spatial density of polonies or clusters that is no less than about 0.1 to about 1 polonies/um³. In some embodiments, the 3D sample can have a spatial density of polonies or clusters that is no less than 0.1 to 1 polonies/um³. In some embodiments, the 3D sample can have a spatial density of polonies or clusters that is no less than 0.002 to 50 polonies/um³. In some embodiments, the 3D sample can have a spatial density of polonies or clusters that is no less than 0.005 to 30 polonies/um³. In some embodiments, the 3D sample can have a spatial density of polonies or clusters that is no less than 0.01 to 50 polonies/um³. In some embodiments, the 3D sample can have a spatial density of polonies or clusters that is no less than 0.05 to 100 polonies/um³.

In some embodiments, such high density 3D sample(s) may be divided into groups as “batches,” and each batch may use a different sequencing primer. In some embodiments, such high density 3D sample(s) may be further divided within each batch into “mini-batches” by selectively turning “off” a mini-batch by controlling administration of fluorescent dyes to be attached to the polonies or clusters. Details of exemplary embodiments of sequencing different batches and/or mini-batches of polonies or clusters are described in PCT patent application Nos. PCT/US23/65972 and PCT/US23/74933 (where the contents are hereby incorporated by reference in their entireties). The methods herein can, in combination with “batch” and/or “mini-batch” sequencing techniques, advantageously allow further increase of base calling throughput by 3×, 5×, 10×, or more than existing methods that sequence the samples in batches without changing the sequencing primers, cartridges, and the optical system.

In some embodiments, the method 600 is performed during a cycle N that is different from a reference cycle. The template image(s) can be generated in the reference cycle(s) and polonies from one or more channels within the reference cycle(s) can be included in the template image in a reference coordinate system, while base calling of cycle N is yet to be performed. In some embodiments, cycle N is the current cycle. N can be any non-zero integer. For example, for short read sequencing, N can be any integer from 1 to 150 or 1 to 300.

FIG. 8B shows a schematic diagram of one or more template images generated in the reference cycle in a reference coordinate system. The template image 210, in some embodiments, include a size that is about identical to a single tile 210 that includes a 5×5 grid of subtiles. In some embodiments, the template image disclosed herein can be individual regions within a subtile. Each template image can include a plurality of polonies or clusters therein.

In some embodiments, the template image can be of about the same size of a flow cell image so that all the polonies, from different tiles, 210 in FIG. 8B, and from multiple channels, can be registered to the same template image. However, such template image may contain polonies that will not be used in at least some operations described herein to reduce computational burden without sacrificing accuracy.

In some embodiments, more than one template images can be generated, and each template image corresponds to at least part of a subtile of a flow cell image from a channel.

The template image herein can be initialized as a virtual image that has a black or dark background with no signals from polonies. For example, the template image can be initialized to be zero or include otherwise minimal image intensity at all pixels.

After the coordinates of a polony is determined by image registration of flow cell images, e.g., across different channels, the intensity of the polony can be added to the template image at the location determined by the coordinates and with the size and shape determined based on registration. The template image can be a virtual image that combines image intensity from polonies obtained from 2, 3, 4, or even more channels at the reference cycle. The pixels of the template containing no polonies in them remains to be black or dark so that the template image can have a cleaner background without noise that appear in actual flow cell images.

The polonies can be from a subtile of flow cell images within a reference cycle, and more specifically, from one or more selected regions of the subtile. The flow cell images can be from different channels of 1, 2, 3, 4, or more channels of the system 100. As a nonlimiting example, a reference cycle can be any cycle of the first 5 or 6 cycles. In some embodiments, the reference cycle can be any cycle that is greater than 0. In some embodiments, the reference cycle is the first cycle.

In some embodiments, the operation 610 comprises generating 2D flow cell images with intensities of polonies or clusters of the 3D sample so that the intensities can be used for performing 3D base calling for different sequencing cycles. The operation of generating the flow cell images can be performed using the sequencing system 110 herein. The operation of generating the flow cell images can be performed using the optical system herein.

The method 600 can comprise an operation of obtaining processed images of the flow cell images. In some embodiments, the operation of obtaining processed images can comprise processing the flow cell images with one or more predetermined processing methods herein.

The one or more predetermined processing methods may include various image processing or operations such filtering, normalization, spatial frequency analysis, etc. In some embodiments, the one or more processing methods can include selecting a kernel and generating the processed images by performing an operation on the flow cell images using the selected kernel. For example, the operation performed can be an opening operation, which can be expressed as f·k, where f is the flow cell image, and k is the kernel. The opening operation can be performed in spatial domain. Alternatively, to make computation faster or less complex, the opening operation may be performed in a different domain, such as the Fourier domain. The opening operation can be the dilation of the erosion of image f by a kernel k. The opening operation can remove objects that are smaller than the kernel, and the subsequent dilation operation may restore the size and shape of the remaining objects. FIGS. 2B and 3B show exemplary processed images after an opening operation. The processed images can also be called opened images in which most of the bright spots of polonies or clusters are removed, and other background objects are retained.

As another example, the operation performed can be a convolution, and the flow cell image can be convoluted with the selected kernel. As yet another example, obtaining the plurality of processed images further comprises: selecting a first kernel and a second kernel; generating first images by convolving the plurality of flow cell images with the first kernel; and generating second images by convolving the plurality of flow cell images with the second kernel. The first images and the second images can be differently blurred images after the convolution with different blurring kernel.

The first or second kernel may take any size that is smaller than the size of the corresponding flow cell image. For example, with an opening operation, the kernel can be 2 by 2, 3 by 3, 4 by 4, 5 by 5, or 6 by 6. In some embodiments, the kernel size can be customized to remove at least some of the noise and unwanted signal that are larger than the kernel size. In some embodiments, the kernel can be circular. The kernel can be other various shapes such as oval, square, rectangular, diamond, etc.

In some embodiments, the kernel is a Gaussian kernel. In embodiments where 2 different kernels are used, the first kernel and the second kernel can be different Gaussian kernels.

In some embodiments, the method 600 can comprise an operation 620 of filtering the flow cell images. The operation 620 of filtering can be based on the processed images generated in its preceding operation. The operation 620 of filtering can be based on a predetermined filter(s). The operation of filtering 620 can generate multiple filtered images, each filtered image corresponding to a flow cell image at a corresponding z level along the axial axis.

In some embodiment, the operation 620 can include subtracting the processed images from a corresponding flow cell image, thereby generating the filtered image. The filtered image can be obtained as fi=f−f·k, where fi represents the filtered image, f represents the flow cell image, and k represent the kernel, and · represents the operation, e.g., opening operation.

In some embodiments, the filtered image is generated in operation 620 based on the corresponding flow cell image before filtering and at least one or more image filters. In some embodiments, the filtered image is an image of the corresponding flow cell image filtered by a predetermined filter. In some embodiments, the predetermined filter is a top-hat filter. In some embodiments, the predetermined filter is a difference of Gaussian (DoG) filter, and the filtered image is an image of the corresponding flow cell image filtered by a difference of Gaussian (DoG) filter. In some embodiments, the predetermined filter(s) may include various filters configured to retain small elements and details (e.g., in-focus polonies or clusters) in the flow cell images and optionally remove elements and details larger than in-focus polonies or clusters.

After filtering, at least part of the noise and undesired signal in the flow cell images are removed, which can include the cell components and out-of-focus polonies. Removal of such noise and undesired signal which are inevitable in 3D flow cell images can advantageously facilitate generating intensities that are attributed to polonies or clusters but no other background objects or noises. Intensities after filtration can be used for more accurate and reliable base calling than those without filtering.

FIGS. 2C and 3C show two filtered images from two different axial locations. FIG. 2C indicates that a larger number of polonies or clusters are in-focus at the first axial location of z=0, and a much smaller number of polonies or clusters are in-focus at a second axial location of z=5, which is about 10 um away from the first axial location.

FIG. 3E shows another exemplary filtered image of the flow cell image in FIG. 3D. The background objects in the flow cell image are filtered out in the filtered image. The out-of-focus polonies in the flow cell image are also removed from the filtered image. The filtered image shows in-focus polonies or clusters.

In some embodiments, the method 600 can include an operation of adding an offset to the filtered image(s). In some embodiments, the offset can be predetermined so that after the offset, the range of image intensity can be within a predetermined range. In some embodiments, different offset can be used to bring different filtered images with various intensities ranges to be within a predetermined range that is common to all the filtered images.

The method 600 can comprise an operation of 630 of generating a maximum intensity projection (MIP) image based on the plurality of filtered images. The operation of 630 can include computing a maximum intensity for each pixel of the MIP image using intensities of the plurality of filtered images at the corresponding pixels. The MIP image can have the same image size, resolution, and/or FOV as the flow cell images. The MIP image can be a flattened 2D image of a stack of flow cell images from different axial locations. The MIP image may correspond to some or all of the FOV of the flow cell images from multiple z levels from a single color channel and at a single flow cycle. The MIP image may correspond to some or all of the FOV of the flow cell images from multiple z levels from a single color channel and at one or more flow cycles.

As an example, an axial stack of 10 flow cell images is acquired at 10 different z levels corresponding to a same color channel in a specific flow cycle. Each z level is about 0.1 um to about 20 um away from its adjacent z location. The first z level is at z=0, and the 10th z level is at z=9. One filtered image is generated for each of the different z locations. A MIP image can be initialized to be the same size as the flow cell images, e.g., 1028 by 1028. All intensity in the initial MIP image can be 0 or any other default initial intensity. For each pixel at pixel (i, j) of the MIP image, image intensities at pixel (i, j) from 10 filtered images are extracted, and a maximum intensity among the 10 different image intensity is selected for pixel (i, j) in the MIP image. In some embodiments, a MIP image can be generated for each cycle corresponding to a same color channel. In some embodiments, a MIP image can be generated for each different channel of 1, 2, 3, 4, 5, 6, or more channels.

In some embodiments, the image intensities in the filtered images are normalized to a predetermined range before the MIP image is obtained using the filtered images. In some embodiments, the MIP images can be normalized across different cycles. In some embodiments, the MIP images for different FOV within the x-y plane, e.g., covering different tiles can be normalized differently to account for spatial variations of sample distribution on the flow cell device. The normalization can be to a predetermined range.

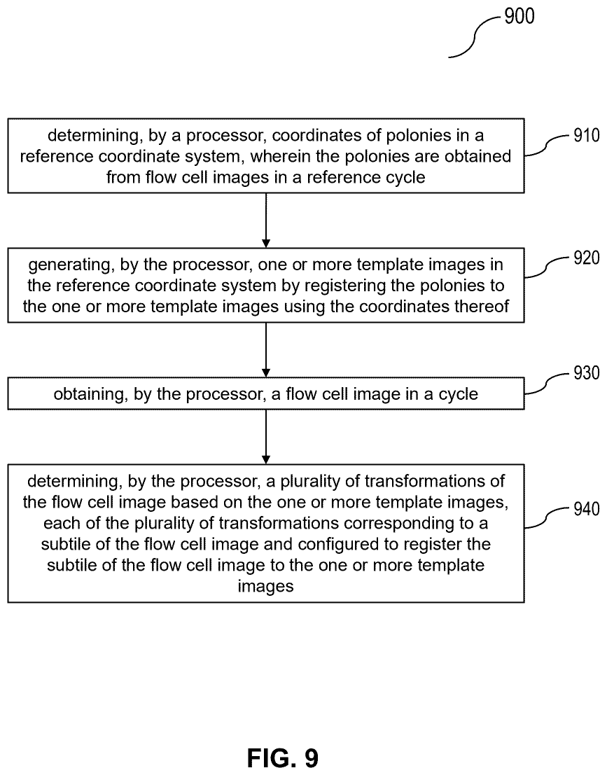

In some embodiments, the method 600 include an operation of image registration of images across different channels and/or different flow cycles. Image registration may be performed after operation 610 but before operation 620. Image registration may be performed after operation 620 but before operation 630. Image registration may be performed after operation 630 but before operation 640. Image registration may be performed before any base calling. Various methods of image registration may be used herein. In some embodiments, the operation of image registration may include one or more operations in method 900 in FIG. 9. Exemplary embodiments of image registration methods are described in PCT patent application No. PCT/US23/67931, where the contents are hereby incorporated by reference in its entirety.

In some embodiments, image registration using existing methods may fail due to the unbalanced nucleotide diversity, which causes one or more flow cell images from some of the color channels to be dimer and/or with fewer brighter spots. Various Image rescue techniques may be used to allow accurate image registration of such flow cell images. Exemplary embodiments of image registration rescue methods are described in PCT patent application No. PCT/US23/67931, where the contents are hereby incorporated by reference in its entirety.

In some embodiments, the method 600 include an operation of registering the MIP images, e.g., 900 in FIG. 9. In some embodiments, the MIP images are registered across channels and/or different cycles before any base calling is performed. Various image registration techniques can be used to register the MIP images. The MIP images can be registered using 2D registration techniques for example, by treating the MIP images as flow cell images acquired from the sequencing system 110. In some embodiments, the MIP images can be registered, e.g., across different channels and/or different cycles, using the image registration method 900 as disclosed herein by treating each MIP image as a flow cell image. In some embodiments, the MIP images can be registered after one or more preprocessing operations disclosed herein are performed.

The method 600 can comprise an operation 640 of performing 3D base calling using the MIP image(s), after image registration of the MIP image(s). The MIP image(s) can be 2D, and the base calling can be performed based on MIP images from different channels per cycle.

In some embodiments, instead of operations 630 and 640, the method 600 may comprise an operation 630′ and 640′ for performing 3D base callings. In some embodiments, the operation 630′ may comprise determining MIPs and their corresponding z levels in the filtered flow cell images. For example, the MIP for flow cell images at 10 different z levels at pixel (10,10) may be 100 in channel 1 and at z=5, and the MIP for pixel (11, 11) may be 110 in channel 4 and at z=6. In some embodiments, the operation 640′ may comprise performing 3D base calling based on MIPs from different channels and with matching z locations, e.g., from channel 1 at z=5 for pixel (10, 10) and from channel 3 at z=6 from channel 3. The base callings may corresponds to different polonies located at different z levels expanding throughout the 3D volume of the example.

In some embodiments, the MIP-based base calling operations may generate fewer number of base calls than the base calling methods using 3D polony maps since some of the polonies may not be included in the MIPs or MIP images.

In some embodiments, the operation 640 of performing base callings using the MIP image comprises performing primary analysis step(s) herein to adjust image intensities of polonies in the MIP; and making base calls for the polonies based on the adjusted image intensities in the MIP. In some embodiments, the primary analysis steps comprise one or more of the following: background subtraction; image sharpening; intensity offset adjustment; color correction; intensity normalization; phasing and prephasing correction; image registration; quality score estimation. In some embodiments, the image registration as a primary analysis step herein is configured to align images from different cycles and different channels, for example, with respect to a template image or a reference coordinate system. In some embodiments, the image registration as a primary analysis step herein is configured to register polonies or clusters from different cycles and different channels, e.g., in the MIP image, to a template image or a reference coordinate system.

For example, the base calling can be performed using the first MIP image from the first color channel and other MIP images from different color channels in cycle N, after the MIP images from different channels are registered relative to the template image(s) disclosed herein. Various existing 2D base calling algorithms can be used. For example, the image intensities at the same pixel in the MIP images from different channels may be compared, and the maximum intensity and its corresponding channel determines the base call of such pixel.

In some embodiments, the method 600 before operation 640, may comprise an operation of determining whether the plurality of flow cell images are of unbalanced diversity or not. Unbalanced diversity may cause error in image registration, color correction or other primary analysis steps leading to inaccurate base calling using existing methods. The methods 600 advantageously enable 3D base calling of flow cell images with low or unbalance diversity. The low or unbalanced diversity may occur in certain regions of the flow cell images (e.g., in one microfluidic channel but not in other microfluidic channels) and/or in one or more flow cycles. The operation of determining whether the plurality of flow cell images are of unbalanced diversity or not in the one or more flow cycles may comprise determining a corresponding percentage of: (1) a number of each type of nucleotide bases, e.g., A, T, C, or G, to (2) a total number of nucleotide bases of a region of the sample immobilized on the flow cell device in at least a region of the FOV, and determining whether the corresponding percentage is less a predetermined diversity threshold or not. The diversity threshold can be customized based on different sequencing application and/or samples. For example, the diversity threshold can be 20%, 18%, 16%, 15%, 12%, 11%, 10%, 8%, 6%, 5%, or less.

In response to determining that the plurality of flow cell images are of unbalanced diversity, e.g., in some regions or in one or more flow cycles, the method 600 comprises an operation of determining image processing parameters based on existing values of corresponding image processing parameters determined in a cycle preceding the one or more cycles. The cycle preceding the one or more cycles may be of balanced nucleotide diversity. For example, cycle 30 has unbalanced diversity and nucleotides A and G has less than 10% of the total number of nucleotides (polonies) in that cycle, preexisting color-correction and/or image registration parameters from cycles that are of balanced diversity, e.g., cycle 29, cycle 25, or even cycle 20 may be used for performing the color correction and/or image registration of cycle 30. As another example, a flow cell image has 16 different regions, and each region may have its corresponding image processing parameters to account for spatial variations of color correction. Further, one region with unbalanced diversity may not affect other regions with balanced diversity in image processing steps such as color correction and/or image registration. Instead, only the region(s) with unbalance diversity can use preexisting image processing parameters of the same region(s) from cycles that are of balanced diversity while the other regions that are not of unbalanced diversity may still use image processing parameters determined in the current cycle. In response to determining that the plurality of flow cell images are of balanced diversity, the methods 600 an operation of determining the image processing parameters for each of the plurality of flow cell images based on the image intensities of the current cycle but not any preceding cycles.

In response to determining that at least some of the plurality of flow cell images at multiple z levels are of unbalanced diversity, e.g., in some regions or in one or more flow cycles, various methods may be used for adjusting the range of image intensity of the flow cell images. For example, identical fiducial markers with predetermined image intensity may be used as reference intensity for adjust image intensities of flow cell images at multiple z levels, before obtaining the MIP image of such flow cell images.

In some embodiments, even the plurality of flow cell images are of unbalanced nucleotide diversity in some regions and/or in one or more flow cycles, the methods 600 and its operations may be configured to perform 3D base calling with a predetermined quality level. The predetermined quality level can be customized based on different sequencing applications. For example, the predetermined quality level may be at least Q30, Q35, Q40 or higher. As another example, the predetermined quality level may be less than 1%, 0.5%, 0.1%, 0.05%, 0.01%, 0.005%, 0.001% or even less errors in the base callings.

The base calling results can be saved with its 3D coordinates. Such 3D coordinates can be used to locate the base calling across different cycles and at different z levels to a common coordinate system. Such 3D coordinates may allow the base callings to be positioned into a 3D volume representing the sample.

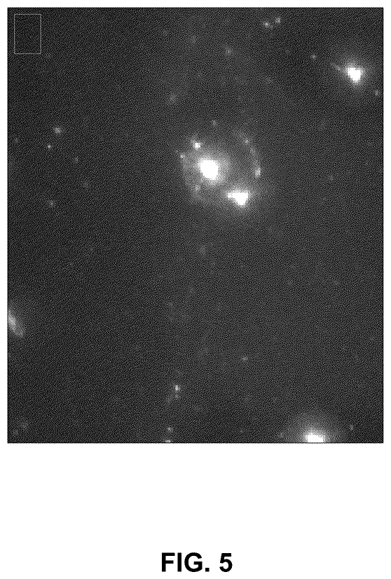

In some embodiments, the method 600 comprise an operation 650 of obtaining a second MIP image based on the plurality of flow cell images. The second MIP is a flattened 2D image of an axial stack of the flow cell images. In some embodiments, the flow cell image are raw images acquired by the sequencing system 110. The raw images herein may include flow cell images that are not filtered with various image filters. The raw images herein may be flow cell images in which both the polonies or clusters and other background information, e.g., noise, cellular components, are retained therein. The operation of obtaining the second MIP can be different from that of the first MIP image. In the flow cell images, e.g., raw images, polonies out-of-focus can have larger full width half maximum (FWHM), so that the signal of the out-of-focus polonies is more spread out than in-focus polonies or clusters. In some embodiments, the larger FWHM can cause a white ring around a polony. FIG. 5 shows an exemplary second MIP image generated directly from the raw flow cell images, and there is a ring or halo round the polony in the center of the image. The ring or halo can be image artifacts that may cause error in base calling. The second MIP can also retain some background information, e.g., from undesired background objects, that may interfere with base calling if the second MIP is used for base calling. As such, the second MIP includes artifacts and/or undesired background objects that require additional processing before accurate and reliable base calling can be make based on the intensities in the second MIP.

In some embodiments, the second MIP can be used to facilitate registration of the first MIP and/or polonies or clusters since it shares the same FOV, resolution, image size, etc., with the first MIP image. The artifacts and/or undesired background objects in second MIP may interfere with base calling if the second MIP is used for base calling, but the same artifacts and/or undesired background objects (that are not in the first MIP) can facilitate registration of first MIP by using the second MIP, for example, for registration to cell images with staining.

In some embodiments, the methods herein advantageously utilize the second MIP for registering the flow cell images to the cell images. In some embodiments, the cell images herein are microscopic images of the sample with staining of some cellular features, such as the membrane and nucleus. The out-of-focus polonies and background objects which may interfere with correct base calling can be used to provide information for registering the flow cell images to the cell images.

In some embodiments, the operation 660 comprises: registering the second MIP image to the cell image. Each color channel may include one or more second MIP images per flow cycle. If image registration of flow cell images across different channels and/or cycles occurs before operation 660, then the second MIP images of the registered flow cell images may be aligned. The second MIP images may be registered to cell images based on the background information contained both within the second MIP images and the cell images. Various methods of image registration may be used to align or register the second MIP images and the cell images. The method 600 can further comprise an operation 660 of performing image registration of the flow cell images based on the second MIP image. In some embodiments, the image registration of the flow cell images to the one or more cell images, e.g., staining images, is in addition to the image registration of the flow cell images across cycles and/or channels. The image registration of the flow cell images may be configured to align the polonies or clusters relative to the cell structures so that base calling can be assigned to relevant cellular components such as the nuclei, membrane, or other regions of the cell. In some embodiments, the operation 660 of registering the flow cell images based on the second MIP image comprises: registering or aligning the background objects in the second MIP to corresponding objects in the one or more cell images. For example, membrane can be traced in the second MIP image using image processing methods like contouring and aligned with the membrane(s) in the cell images.

In some embodiments, the operation 660 of performing image registration of the plurality of flow cell images based on the second MIP image comprises: registering the second MIP image to the template image or the reference coordinate system.

In some embodiments, the second MIP and the first MIP are obtained from flow cell images of the same FOV, thus the second and first MIP image may share the same FOV. In some embodiments, registering the second MIP image to the template image or the reference coordinate system can rely on the image registration information of the first MIP to the template image. In some embodiments, registering the first MIP image to the cell images can be based on the registration of the second MIP image to the cell images since the first and second MIP images share the same FOV.

In some embodiments, the method 600 can comprise an operation 670 of registering 3D base calling to the one or more cell images. In some embodiments, the operation 670 may comprise: registering the first MIP image to the one or more cell images. In some embodiments, the operation 670 may comprise: registering the first MIP image to the one or more cell images based on the second MIP images since the second MIP images contain background information that may facilitate registration to the one or more cell images. In some embodiments, the operation 670 may comprise: registering the 3D base calling based on the registered first MIP image and the second MIP image to one or more cell images. The background objects in the second MIP can be used to align the second MIP and the first MIP to the cell images. The cell images can have at least some identical background objects as those in the second MIP, but with possible transformation, e.g., translation and/or rotation. The transformation may be represented by a single transformation of the whole image or by separated into multiple transformations, each transformation representing a portion of the whole image. After finding the transformation(s) of the background objects between the second MIP and the cell images, the polonies and clusters can be registered to the cell images.

In some embodiments, the operation 670 of registering the first MIP image and/or the 3D base calling to the cell images may not rely on the second MIP image(s) obtained directly from the flow cell image but only on the first MIP image(s) of the filtered images. In some embodiments, instead of using the second MIP generated from the flow cell images, the flow cell images can be directly used for registering the filtered images to the cell images. The flow cell images may contain noise and undesired background objects for base calling purpose but such noise and background objects can facilitate registering the flow cell images and the filtered images to cell images.

In these embodiments, instead of operation 660 using the second MIP image, the method 600 may comprise an operation 660′ of using background information obtained from one or more of: the first MIP image(s), the opened images/processed images, the filtered images, and the flow cell images for registration with the cell images.

The background objects can be used to align one or more of: the first MIP image(s), the opened images/processed images, the filtered images, and the flow cell images to the cell images by using one or more transformations. The background objects can be used to align the first MIP image to the cell images by using one or more transformation(s). The transformation may be represented by a single transformation of the whole image or be separated into multiple transformations, each representing a portion of the whole image. After finding the transformation(s) of the background objects between the first MIP and the cell images, the polonies and clusters can be registered to the cell images.

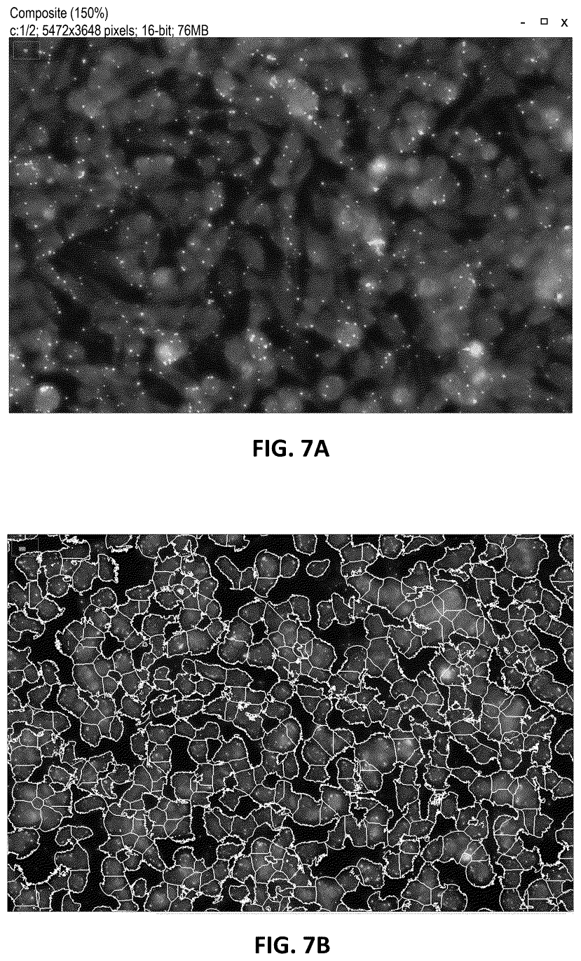

In some embodiments, the operation 670 of registering the MIP image and/or the 3D base calling to the cell images may not rely on the second MIP image(s) but instead based on fiducial markers. In some embodiments, instead of operation 660 using the second MIP image, the method 600 may comprise an operation 660″ of using fiducial markers external to the samples for registration to the cell images. The same fiducial marks may exist in one or more of: the first MIP image(s), the opened images/processed images, the filtered images, and the flow cell images. Such fiducial markers can also be included in the cell images. Aligning the same fiducial markers can generate the transformation(s) between the sequencing images, e.g., the first MIP image(s) or the flow cell images, to the cell images. The transformation(s) can be used to register or align polonies or clusters between the flow cell images and the cell images. FIGS. 7A-7B show exemplary registered first MIP image overlayed on the corresponding cell image with cellular structures. The first MIP image includes bright spots representing polonies. Some of the bright spots overlap with the stained nuclei, while some other bright spots occur within the cell membrane but outside of the nuclei. FIG. 7B shows segmentation of individual cells so that polonies or clusters can be grouped with respect to each individual cell.

### Base Calling Using 3D Polony Maps

FIG. 6B shows a flow chart of an exemplary embodiment of a computer-implemented method 600 for performing 3D base calling from the flow cell images using the 3D polony map(s). The method 600 can include some or all of the operations disclosed herein. The operations may be performed in but is not limited to the order that is described herein.

The method 600 can be performed by one or more processors disclosed herein. In some embodiments, the processor can include one or more of: a processing unit, an integrated circuit, or their combinations. For example, the processing unit can include a central processing unit (CPU) and/or a graphic processing unit (NPU). The integrated circuit can include a chip such as a field-programmable gate array (FPGA). In some embodiments, the processor can include the computing system 400.

The method 600 can be performed based on flow cell images from a current sequencing cycle alone or in combination with information from preceding cycle(s) of the current sequencing cycle.

In some embodiments, some or all operations in method 600 can be performed by the FPGA(s). In embodiments when some operations are performed by FPGA(s), the data after an operation performed by the FPGA(s) can be communicated by the FPGA(s) to the CPU(s) so that CPU(s) can perform subsequent operation(s) in method 600 using such data. Similarly, data can also be communicated from the CPU(s) to the FPGA(s) for processing by the FPGA(s). In some embodiments, all the operations in method 600 can be performed by CPU(s). Alternatively, the operations performed by CPU(s) can be performed by other processors such as the dedicated processors, or NPU(s). In some embodiments, all the operations in method 600 can be performed by FPGA(s) and/or NPU(s).

The method 600 can comprise an operation 610 of obtaining multiple flow cell images of one or more samples. The flow cell images can be acquired at different locations along an axial axis, i.e., z axis. In some embodiments, the operation 610 comprises actively retrieving or passively receiving multiple flow cell images of a sample to be processed. In some embodiments, the operation 610 comprises acquiring the flow cell images using the imager 116 of the sequencing system. In some embodiments, the flow cell images are acquired by a NGS sequencing system.

The sample can be in 3D. The sample can be a volumetric sample that may contain different biological information at the same x-y location but different z location. The sample can be an in situ sample. The sample can include multiple cells, tissue, or their combination. The sample can be any biological sample that has a thickness that is greater than a predetermined threshold (e.g., depth of field) along the axial axis. The sample can be any biological sample that has a thickness that is greater than the depth of field of the optical system. For example, the thickness can be greater than 1 um, 2 um, 3 um, 4 um, 5 um, 8 um, 10 um, 12 um, 15 um, 20 um, or more. The z axis (e.g., axial axis) can be orthogonal to the image plane defined by x and y axes, as show in FIG. 8B.

In some embodiments, the flow cell images can include a first plurality of flow cell images and a second plurality of flow cell images. Each of the first plurality of flow cell images and the second plurality of flow cell images can be acquired at a corresponding location along an axial axis or a z axis. The axial axis can extend from an objective lens of the sequencing system 110 to the sample located on the flow cell positioned on the sequencing system. As a nonlimiting example, the first plurality of flow cell images can be obtained from the reference cycles and the second plurality of flow cell images can be obtained from one or more cycles different from the reference cycles. As another example, the first plurality of flow cell images can be identical to the second plurality of flow cell images.

The flow cell images can be acquired using the optical system disclosed herein, from the 1, 2, 3, 4, or more channels of the imager 116. Each flow cell image can include one or more tiles (imaging areas), and each tile can be divided into multiple subtiles. Each subtile can include a plurality of polonies or clusters. Each subtile can include multiple regions with each region including a number of polonies. For example, the polonies can be extracted from corresponding regions of flow cell images from 4 different channels in a given cycle. As another example, the polonies can be extracted from flow cell images from a single channel. The flow cell image as disclosed herein can be an image that is acquired from a flow cell 112 as shown in FIG. 1.

In some embodiments, a flow cell image herein can be an image of one or more tiles, one or more subtiles, one or more segmented regions with tile(s) or subtile(s), or their combinations. Each flow cell image can comprise a field of view (FOV). The FOV can be orthogonal to the axial axis. The FOV can be within the x-y plane. The FOV of different flow cell images at different axial locations can be identical within the x-y plane. The FOV of different flow cell images at different axial locations can have at least an overlapping portion within the x-y plane.

The image resolution of different flow cell images at different axial locations can be about identical or exactly identical. In some embodiments, the image resolution of different flow cell images at different axial locations is different.

FIGS. 2A and 3A show two exemplary flow cell images acquired at two different z levels along the axial axis of a same 3D sample within the same sequencing cycle.

Each flow cell image at a specific z level includes intensities generated by polonies and clusters at the corresponding z location. As shown in FIGS. 2A-3A, signals from polonies and clusters are small bright spots within the images. Each bright spot can be of various sizes that is less than a couple of pixels, e.g., less than a pixel, about a pixel, about 2 pixels, 3 pixels, 4, pixels, 5 pixels, or more. In some embodiments, each signal spot of the polonies or clusters can be any number of pixels in the range from 0.01 pixel to about 72 pixels. In some embodiments, each signal spot of the polonies or clusters can be any number of pixels in the range from 0.1 pixel to about 16 pixels.

The polonies can be from a subtile of flow cell images within a reference cycle, and more specifically, from one or more selected regions of the subtile. The flow cell images can be from different channels of 1, 2, 3, 4, or more channels of the system 100. As a nonlimiting example, a reference cycle can be any cycle of the first 5 or 6 cycles. In some embodiments, the reference cycle can be any cycle that is greater than 0. In some embodiments, the reference cycle is the first cycle.

In some embodiments, the flow cell images are acquired at one or more reference cycles or a cycle different from the one or more reference cycles. As a nonlimiting example, cycles 1-5 or 2-5 can be reference cycles. The flow cell images can be acquired within a single cycle or a couple of cycles.

Each flow cell image can include intensities generated by the cell and its structural elements. Such structural elements can be background objects or components. FIG. 3D show multiple cells and the polonies or clusters as small bright spots are generally within the contours of different cells.

In some embodiments, when the focus of the optical system includes a range, e.g., 0.1 um, 0.2 um, 0.3 um, 0.5 um, 0.6 um, 0.8 um, 1 um, 2 um, 3, um, 4 um, 5 um, etc. expanding along z axis. Polonies and clusters that are within the range of focus can appear in-focus or about in-focus in the flow cell image. Flow cell images at a specific z level can also include signals from polonies and clusters that are not within the focus range of the image, but at different z locations. So, such polonies or clusters are out-of-focus. As shown in FIG. 3A, bigger and blurred signal spots represent out-of-focus polonies or clusters. Some of the out-of-focus polonies or clusters are circled in FIG. 3A.

Each flow cell image at a specific z level can also include noises caused by the optical system and/or undesired signal from the sample. The undesired signal can be signal coming from components of the sample such as membrane, cytosol, and mitochondria. Such background objects can be any objects, relatively larger in size than the polonies or clusters. As shown in FIG. 3A, there is a blurry cellular contour (at the arrows) in the flow cell image, and most of the signal spots are contained within the blurry contour. In some embodiments, background objects can include any objects within the 3D sample but are not polonies or clusters.

In some embodiments, the polonies or clusters being sequenced in a flow cycle may have a certain nucleotide diversity, e.g., in base calling. The methods 600 may allow 3D base calling of flow cell images even if the polonies or clusters are of low or unbalanced diversity in sequencing cycle(s). The nucleotide diversity of a population of nucleotide acid molecules, e.g., polonies or clusters, can refer to the relative proportion of nucleotides A, G, C, and T/U that are present in each flow cycle. The relative proportion of nucleotides may be within a region of the field of view or within the entire flow cell image. An optimally high or balanced diversity data can generally have approximately equal proportions of all four nucleotides represented in each flow cycle of a sequencing run. A low or unbalanced diversity data can generally include a high proportion of certain nucleotides and low proportion of other nucleotides in some flow cycles of a sequencing run, e.g., less than 10% of the total number of all 4 nucleotides. As a result, images corresponding to the high portion of certain nucleotides can have more signal spots (polonies or clusters) than images corresponding to the low portion of certain nucleotides. As an example of low or unbalanced diversity data, the bases A, T, C, G can be about 1%, about 2%, about 1%, and about 95%, respectively, of the total number of polonies, in a certain flow cycle. Subsequently, the flow cell images from channels corresponding to A, T, and C in this particular flow cycle are darker and/or with much fewer polonies or clusters than the flow cell image corresponding to nucleotide G. As another example of low or unbalanced diversity data, the bases A, T, C, G in polonies in multiple flow cycles can be about 2%, about 5%, about 10%, and about 83%, respectively. In embodiments where low or unbalanced diversity data is present in a particular cycle and is imaged for sequencing analysis, image registration and subsequent base calling using existing technologies may fail because image(s) from one or more channels are too dark (e.g., signal spots of polonies are too sparse and/or dim) comparing with images acquired from other channels thereby causing problems in sequencing analysis. Further, in embodiments where low or unbalanced diversity data is present in a particular cycle, existing methods may fail to identify polonies or clusters that are dimer or sparser from other background information, e.g., cellular structural. In some embodiments, the methods 600 are configured to perform base calling of 3D samples even if the polonies or clusters are of low diversity in some regions of the flow cell during some flow cycles from the 3D sample.

In some embodiments, the method 600 is performed at least partly during a cycle N that is different from a reference cycle. The template image(s) and/or the 3D polony map can be generated in the reference cycle(s) and polonies from one or more channels within the reference cycle(s) can be included in the template image(s) and/or the 3D polony map in a reference coordinate system, while base calling of cycle N is yet to be performed. In some embodiments, cycle N is the current cycle. N can be any non-zero integer. For example, for short read sequencing, N can be any integer from 1 to 150.

FIG. 8B shows a schematic diagram of one or more template images generated in the reference cycle in a reference coordinate system. The template image 210, in some embodiments, include a size that is about identical to a single tile 210 that includes a 5×5 grid of subtiles. In some embodiments, the template image disclosed herein can be individual regions within a subtile. Each template image can include a plurality of polonies therein.

In some embodiments, the template image can be of about the same size of a flow cell image so that all the polonies, from different tiles, 210 in FIG. 8B, and from multiple channels, can be registered to the same template image. However, such template image may contain polonies that will not be used in at least some operations described herein to reduce computational burden without sacrificing accuracy.

In some embodiments, more than one template images can be generated for the same axial location, and each template image corresponds to at least part of a subtile of a flow cell image from a channel.

The template image herein can be initialized as a virtual image that has a black or dark background with no signals from polonies. For example, the template image can be initialized to be zero or include otherwise minimal image intensity at all pixels.

After the coordinates of a polony is determined by image registration of flow cell images, e.g., across different channels, the intensity of the polony can be added to the template image at the location determined by the coordinates and with the size and shape determined based on registration. The template image can be a virtual image that combines image intensity from polonies obtained from 2, 3, 4, or even more channels at the reference cycle. The pixels of the template containing no polonies in them remains to be black or dark so that the template image can have a cleaner background without noise that appear in actual flow cell images.

In some embodiments, the method 600 comprises an operation 610 of generating an axial stack of 2D flow cell images. The operation 610 may be identical in two different embodiments of the methods 600 in FIGS. 6A-6B. The flow cell images can include intensities of polonies or clusters from a 3D sample. The intensities can be used for performing 3D base calling for different sequencing cycles. The operation of generating 2D flow cell image can be performed by the sequencing system 110 herein.

The method 600 can comprise an operation of generating the processed images of the flow cell images. In some embodiments, the operation of generating the processed images can comprise processing the flow cell images with one or more predetermined processing methods.

In some embodiments, the one or more processing methods can include selecting a kernel and generating the processed images by performing an operation on the flow cell images using the selected kernel. For example, the operation performed can be an opening operation, which can be expressed as f·k, where f is the flow cell image, and k is the kernel. The opening operation can be performed in spatial domain. Alternatively, to make computation faster or less complex, the opening operation may be performed in a different domain, such as the Fourier domain. The opening operation can be the dilation of the erosion of image f by a kernel k. The opening operation can remove objects that are smaller than the kernel, and the subsequent dilation operation may restore the size and shape of the remaining objects. FIGS. 2B and 3B show exemplary processed images after an opening operation. The processed images can also be called opened images in which most of the bright spots of polonies or clusters are removed, and other background objects are retained.

As another example, the operation performed can be a convolution, and the flow cell image can be convoluted with the selected kernel. As yet another example, obtaining the plurality of processed images further comprises: selecting a first kernel and a second kernel; generating first images by convolving the plurality of flow cell images using the first kernel; and generating second images by convolving the plurality of flow cell images using the second kernel. The first images and the second images can be different blurred images after the convolution with different blurring kernel.

The first or second kernel may take any size that is smaller than the size of the corresponding flow cell image. For example, with an opening operation, the kernel can be 2 by 2, 3 by 3, 4 by 4, 5 by 5, 6 by 6 pixels. In some embodiments, the kernel size can be customized to remove at least some of the noise and unwanted signal that are larger than the kernel size. In some embodiments, the kernel can be circular. The kernel can be other various shapes such as oval, square, rectangular, diamond, etc.

In some embodiments, the kernel is a Gaussian kernel. In embodiments where 2 different kernels are used, the first kernel and the second kernel can be different Gaussian kernels.

In some embodiments, the method 600 can comprise an operation 620 of filtering the flow cell images. The operation 620 may be identical in two different embodiments of the methods 600 in FIGS. 6A-6B. The operation 620 of filtering can be based on the processed images generated in its preceding operation. The operation 620 of filtering can be based on a predetermined filter(s). The operation of filtering 620 can generate multiple filtered images, each filtered image corresponding to a flow cell image at a corresponding z level along the axial axis.

In some embodiment, the operation 620 can include subtracting the processed images from a corresponding flow cell image, thereby generating the filtered image. The filtered image can be obtained as fi=f−f·k, where fi represents the filtered image, f represents the flow cell image, and k represent the kernel, and · represents the operation e.g., opening operation.

In some embodiments, the filtered image is generated in operation 620 based on the corresponding flow cell image before filtering and at least one or more image filters.

In some embodiments, the filtered image is an image of the corresponding flow cell image filtered by a predetermined filter. In some embodiments, the predetermined filter is top-hat filter. In some embodiments, the predetermined filter is a difference of Gaussian (DoG) filter, and the filtered image is an image of the corresponding flow cell image filtered by a difference of Gaussian (DoG) filter.

In some embodiments, the predetermined filter(s) may include various filters configured to retain small elements and details (e.g., in-focus polonies or clusters) in the flow cell images and optionally remove elements and details larger than in-focus polonies or clusters.

After filtering, at least part of the noise and undesired signal in the flow cell images are removed, which can include the cell components and out-of-focus polonies. Removal of such noise and undesired signal advantageously facilitate generating intensities that are attributed to polonies or clusters but not other background objects or noises. Intensities after filtration can be used for more accurate and reliable base calling than those without filtering.

FIGS. 2C and 3C show two filtered images from two different axial locations. FIG. 2C indicates that a larger number of polonies or clusters are in-focus at the first axial location of z=0, and a much smaller number of polonies or clusters are in-focus at a second axial location of z=5, which is about 10 um away from the first axial location.

FIG. 3E shows another exemplary filtered image of the flow cell image in FIG. 3D. The background objects in the flow cell image are filtered out in the filtered image. The out-of-focus polonies in the flow cell image are also removed from the filtered image. The filtered image shows in-focus polonies or clusters.

In some embodiments, the method 600 can include an operation of adding an offset to the filtered image(s). In some embodiments, the offset can be predetermined so that after the offset, the range of image intensity can be within a predetermined range. In some embodiments, different offset can be used to bring different filtered images with various intensities ranges to be within a predetermined range that is similar to all the filtered images.

In some embodiments, the processed images include the first and second pluralities of processed images, and the filtered images include the first and second pluralities of filtered images. In some embodiments, the first plurality of processed images and the first plurality of filtered images are from the one or more reference cycles and different channels. In some embodiments, the second plurality of flow cell images, the second plurality of processed images and the second plurality of filtered images are from the one or more reference cycles and the different channels. In some embodiments, the second plurality of flow cell images, the second plurality of processed images and the second plurality of filtered images are from one or more cycles different from the one or more reference cycles and from different channels. In some embodiments, the first and second plurality of flow cell images are identical, the first and second plurality of processed images are identical, and the first and second plurality of filtered images are identical.

The method 600 can comprise an operation 635 of obtaining the 3D polony map. The 3D polony map include corresponding 3D locations of polonies or clusters, and the 3D polony map may be used as a road map for locating all the possible polonies in 3D for 3D base calling. In other words, base calling is performed only on polonies that are included in the 3D polony map. The 3D polony map may include all of the polonies of the sample(s) that are within the 3D FOV. In some embodiments, the 3D polony map may include only some of the polonies of the sample(s) that are within the 3D FOV.

The 3D polony map may be determined in one or more reference cycles. During a cycle different from the reference cycle(s), the 3D polony map may have been pre-generated, e.g., in the reference cycle(s), and can be obtained by actively requesting or retrieving or passively receiving the 3D polony map. In some embodiments, a single 3D polony map is used during sequencing analysis of the same 3D sample so that in cycles different from the reference cycles, no new 3D polony map needs to be generated. In some embodiments, the 3D polony map may be updated after a number of cycles during sequencing of the same 3D sample.

Continuing referring to FIG. 6B, the method 600 can comprise an operation 635 of generating the 3D polony map. The operation of generating the 3D polony map can but is not limited to occur during one or more reference cycles. As a nonlimiting example, cycles 1-5 or 2-5 can be reference cycles. In some embodiments, the 3D polony map generated in the one or more reference cycles can be used in any cycles different from the reference cycles so that additional computational complexity, cost, and time for generating 3D polony maps in every cycle can be avoided.

The 3D polony map can be generated based on the one or more 2D template images. Each of the 2D template image corresponds to the flow cell image(s) at a specific z location. Each of the 2D template image corresponds to the flow cell image(s) at the specific z level and at a specific tile or subtile. Each of the 2D template image may correspond to one or more sequencing cycles. Each template image may correspond to one or multiple channels. For example, if there are 10 different z levels covering the 3D sample being sequenced, there can be 10 2D template images corresponding to the different z levels for a single tile or subtile during cycle N. The 2D template images may be in the same coordinate system as the 2D polony maps. The 2D template images may contain exactly identical polonies as the 2D polony map. In some embodiments, the 2D template images may contain different polonies from the 2D polony map since the template image may cover a smaller region than a 2D polony map to reduce computational complexity and save time in sequencing analysis. In some embodiments, the 2D template images may contain different polonies from the 2D polony map since the template image may contain duplicate polonies that may not be included in the 2D polony map.

A polony map herein, either 2D or 3D, can be saved as a list of coordinates. Each entry in the list of coordinates can correspond to a polony, for example, a center of the polony. Instead of saving a 2D or 3D matrix as the polony map, the list of coordinates can be stored with much less storage space, and can be utilized more efficiently in computations.

In some embodiments, the operation 635 of generating the 3D polony map can comprise obtaining the 2D template images(s). Obtaining the template images can comprise generating the template images or receiving or retrieving the template images. The 2D template images can be generated using various methods including the methods and operations described herein. The 2D template images can include polonies with subpixel resolution. Exemplary embodiments of obtaining the 2D template image are disclosed in U.S. patent application Ser. Nos. 18/078,797 and 18/078,820 (where the contents of which are hereby incorporated by reference in their entireties). The 2D template images can be generated after filtered, e.g., by the top hat filter or the DoG filter. The 2D template images can be generated after filtering the flow cell images and registering the filtered images to a reference coordinate system. The 2D template images can be generated in one or more reference cycles and the same template images can be used across different cycles and channels. The 2D template image can include a list of coordinates of polonies. For example, each entry in the list can be 2D or 3D coordinates of the polonies.

In some embodiments, the operation of generating the 3D polony map can comprise combining the one or more 2D template images into a candidate 3D polony map. For example, the lists of coordinates in the 2D template images can be added together.

In some embodiments, the operation of generating the 3D polony map may comprise combining the one or more 2D polony maps into a candidate 3D polony map. In some embodiments, the operation of generating the 3D polony map may comprise combining the one or more 2D template images into a candidate 3D polony map. In some embodiments, the 2D template images and the 2D polony map may be different in excluding duplicate polonies (in 3D) using only 2D information. In some embodiments, the operation of generating the 3D polony map may comprise combining one or more 2D polony maps or one or more 2D template images into a candidate 3D polony map depending on which ones include a more complete set of possible polonies of the sample to avoid excluding polonies incorrectly before removal of duplicate polonies.

In some embodiments, the 3D polony map is saved as a list of entries, each entry representing a polony and including spatial and intensity information of that polony. In some embodiments, when the 3D polony map is saved as a 3D matrix instead of a list of entries, the operation of generating the 3D polony map can comprise extracting polonies in the one or more 2D template images. Instead of directly combining the 2D template images, the polonies in the one or more template images can be extracted, and the extracted polonies can be included in the candidate 3D polony map based on their coordinates in the template images.

The candidate 3D polony map may include a 3D resolution determined by the resolution in the image plane, i.e., x-y plane, and the resolution along z axis. The resolution along z axis may be determined by the slice thickness of the flow cell images. The resolution along z axis may be determined by the depth of field of the optical system so that the polonies within the depth of field can be in-focus. In some embodiments, subpixel resolution along x, y, and/or z can be achieved. In some embodiments, subpixel resolution that is 2×, 4×, 5×, 6×, 8×, 10× higher than the pixel resolution along the corresponding axis. For example, the pixel resolution may be 3 um, 10× subpixel resolution is 0.3 um. The subpixel resolution may be achieved using the methods disclosed in U.S. patent application Ser. Nos. 18/078,797 and 18/078,820 (where the contents of which are hereby incorporated by reference in their entireties). In some embodiments, the subpixel resolution may be achieved based on the pixel resolution at different z levels using various interpolation methods, so that the in-focus polonies location may be between two immediately adjacent z levels determined using interpolation of the pixel intensities at the corresponding pixel, e.g., pixel (x1,y1) at z1, and pixel (x1, y1) at z2, of the two immediately adjacent z levels, optionally with pixel intensities from other z levels, e.g., at z=0 and z=3.

The candidate 3D polony map can be initialized with 0 or other predetermined intensity values in all its pixels at different z locations. The extracted intensities can be used to replace the initial value in the candidate 3D polony map to indicate the pixels or voxels that is at least part of a polony. As a nonlimiting example, the 3D polony map can be a 3D matrix of 0 and 1, where each pixel with 1 indicates that pixel that is part of a polony.

A single polony in the 3D sample may appear in one, two, or even more z levels so that the same polony may be included in multiple flow cell images at different z locations. For example, a polony at (x1, y1) at the location z1 may be included again at (x1-1, y1-1) at the location z2. As such, the candidate 3D polony map can include duplicate polonies. The duplicate polonies need to be removed for accurate and reliable 3D base calling. The operation 635 of generating the 3D polony map can comprise removing duplicate polonies from the candidate 3D polony map.

To remove duplicate polonies, the operation 635 may comprise: performing preliminary base callings. The location of polonies for base callings can be determined by the 2D template image(s) while the intensities for performing base callings can be extracted from the filtered images obtained in operation 620. Similarly, the location of polonies for base callings can be determined based on the candidate 3D polony map which may include all the 2D template images. The filtering herein can advantageously remove intensity interferences from out-of-focus polonies and background objects, so that the intensities can be used for more accurate and reliable base calling than the unfiltered flow cell images. In some embodiments, the 2D template images contains coordinates of polonies in a reference coordinate system so that even if the polonies may have shifted across cycles, the base callings can still be attributed to the same polonies.

After the preliminary base callings are obtained, the operation 635 can include an repetitive operation of removing the duplicate polonies from the 2D template images or from the candidate 3D polony map until a stopping criteria is met. The repetitive operation can include identifying candidate polonies with an identical base call. In response to identifying at least two candidate polonies with an identical base call, the candidate polonies may contain zero, one, two, or more duplicate polonies. The operation 635 can further comprise an operation of determining 3D distance between each pair of polonies among the candidate polonies. For each pair of polonies (non-repetitive pair), the 3D distance can be calculated based on the coordinates of the polonies. The coordinates can be 3D. The coordinates can include the 2D coordinates of the polonies, e.g., after registration in the reference coordinate system, and the z level of polonies. The 3D distance can be in pixels. The 3D distance can be in other units, e.g., um. The 3D distance can be used to determine if the two polonies with identical base calls are in proximity to each other or not.

In response to determining that the 3D distance between the two polonies is within a predetermined distance threshold, the operation 635 can include an operation of determining the image intensity for each of the two polonies from the filtered images or the template images. In some embodiments, determining the image intensity for each of the polonies can include normalizing and/or offsetting the image intensity at different z levels to a predetermined range. For example, the normalization and/or offsetting can be based on intensities of fiducial markers at the different z locations. If two identical fiducial markers at two different z levels are of different intensities, normalization and/or offsetting may be used to bring the two different intensities to the same level. Such normalization and/or offsetting may then be applied to the image intensities of polonies at the corresponding z levels as the fiducial markers. Subsequently, the operation 635 can include removing a polony of the two with a smaller image intensity. The two polonies within the predetermined distance threshold can be considered as a same polony that is duplicated. The duplicate with smaller intensity can be more out-of-focus than the one with greater intensity, and can be removed to ensure accurate and reliable base calling. The predetermined distance threshold can be customized based on the characteristics of the sample, the imaging parameters, the polonies, etc. For example, the predetermined distance threshold can be based on a depth of field of an optical system, a distance between two adjacent flow cell images along an axial direction, or a combination thereof. The distance threshold alone or combination with the stopping criteria, can be adjusted to balance the true duplicates and the false duplicates that are removed. As an example, polony p1 has a preliminary base calling of A determined using its image intensity after filtering, and its location after registration to the reference coordinate system can be at (xp1, yp1). Candidate polonies can be determined to be those with the same base calling of A. 3D distance from each candidate polony to polony p1 can be calculated and compared to a predetermined threshold, e.g., of 0.5 um. Polony p2 that satisfies the distance threshold can be considered as a possible duplicate of p1. Intensities of p1 and p2 are compared and polony p2 with a greater intensity is retained while the coordinates of polony p1 is removed as a duplicate.

After the duplicate polonies are removed from the 2D template images or the candidate 3D polony map, the 3D polony map is generated which includes all the polonies and corresponding coordinates that base calling can be performed on. The 3D polony map can include location information of such polonies. For example, the 3D polony map may include coordinates of each polony in the corresponding filtered images. The 3D polony map may further include the z level of each polony, the z level may be the center location of the polony which may expand more than several pixels. The 3D polony map may include size and/or shape of the polonies. The 3D polony map may include an unique identification of each polony. The 3D polony map may include image intensity of polonies. Such image intensity may be filtered intensities obtained from the filtered images disclosed herein.

The stopping criteria herein can be customized. For example, the stopping criteria can be based on different type of samples, imaging parameters, size and shape of polonies, etc. The stopping criteria, the distance threshold, or both may be adjusted to balance the true duplicates and the false duplicates that are removed. As a nonlimiting example, the stopping criteria can be removing the first 100 duplicate polonies. As a nonlimiting example, the stopping criteria can be performing removal of the duplicates only within a selected time window. As yet another example, the stopping criteria can be there is no duplicate which satisfies the predetermined distance threshold.

Details of exemplary embodiments of methods for generating the 3D polony map are described in U.S. patent application Ser. Nos. 18/078,797 and 18/078,820 (where the contents of which are hereby incorporated by reference in their entireties).

In some embodiments, the method 600 include an operation of registering the flow cell images, the processed images, and/or the filtered images. In some embodiments, the images are registered across channels and different cycles. In some embodiments, the images are registered before any base calling are performed. In some embodiments, the images are registered across channels and different cycles before generating the template images or obtaining the 3D polony maps. In some embodiments, the images are registered across channels and different cycles before generating the filtered images or the processed images.

Various image registration techniques can be used to register the images. The images can be registered using 2D registration techniques. When registering the processed images or the filtered images, they can be treated as flow cell images during registration. In some embodiments, the images can be registered, e.g., across different channels and/or different cycles, using the image registration method 900 as disclosed herein by treating each image to be registered as a flow cell image that can be acquired using the sequencing system 110. Exemplary embodiments of image registration methods are described in PCT patent application No. PCT/US23/67931 (where the contents are hereby incorporated by reference in its entirety).

In some embodiments, the images can be registered after one or more preprocessing operations disclosed herein are performed. In some embodiments, the operation of registering the flow cell images, the processed images, and/or the filtered images may occur before the operation 635 of obtaining a 3D polony map.

In some embodiments, the operation of registering the flow cell images, the processed images, and/or the filtered images is with respect to a reference coordinate system. In some embodiments, the operation of registering the flow cell images, the processed images, and/or the filtered images is with respect to one or more template images. The operation of registering the images can comprise generating the one or more template images in a reference coordinate system. In some embodiments, the operation of registering the images can comprise registering polonies to template polonies in the one or more template images. The operation of registering the images can comprise determining a plurality of transformations based on the one or more template images. Each of the plurality of transformations can corresponds to a corresponding subtile of the flow cell images, the processed images, or the filtered images and configured to register the subtile to the one or more template images. Each transformation can be used to register a corresponding subtile or tile to the one or more template images. The plurality of transformations can comprise one or more affine transformations.

In some embodiments, the operation of registering the images can comprise performing image registration of the polonies based on fiducial markers. The fiducial markers can be located on the flow cell. Alternatively, the fiducial markers can be external to the flow cell.

In some embodiments, the image registration as a primary analysis step herein is configured to align images from different cycles and/or different channels, for example, with respect to a template image or a reference coordinate system. In some embodiments, the image registration as a primary analysis step herein is configured to register polonies or clusters from different cycles and different channels, e.g., in the filtered image, to a template image or a reference coordinate system.

For example, the base calling can be performed using the filtered images from different channels in cycle N after the filtered images from different channels are registered relative to the corresponding template image disclosed herein.

The method 600 can comprise an operation 645 of extracting polony intensities based on the 3D polony map. For each polony in the 3D polony map, the location information of such polony can be obtained from the 3D polony map, e.g., 2D coordinates of the polony and the z location. Using the 2D coordinates and the z location, the corresponding filtered image and its pixel(s) can be determined. Image intensity of such pixels can be extracted from the corresponding filtered image as intensity of such pixel for performing base calling.

In some embodiments, multiple neighboring polonies may be at least partially overlapping with each other in the 3D polony map and they may not be resolved along x, y, and/or z directions using existing methods. In some embodiments, the operation 645 and/or operation 655 herein advantageously utilize predetermined base call information to determine the polony intensities of neighboring polonies that are too close to each other (e.g., partially overlapping). The predetermined base call information may include expected image intensities from 2 or more channels. The predetermined base call information may include expected image intensities from two or more cycles. The predetermined base call information can be used to decompose at least partially overlapped polonies and their intensities by determining a linear combination of the measured intensities of the overlapped polonies to match the expected pixel intensities given possible DNA sequences, e.g., barcodes. For example, in the first color channel, the obtained image intensity is 0, 1, 3 in 3 consecutive cycles, and the observed intensity is 1, 2, 0 in the second color channel in the same cycles at a same pixel (m,n). The expected signal intensities include: 0, 1, 1 in channel 1 and 1, 0, 0 in channel 2 for a first sequence of nucleotide bases, e.g., a first barcode sequence, in the same 3 cycles; and the expected signal intensities also include: 0, 0, 1 in channel 1, and 0, 1, 0 in channel 2 for a second sequence of nucleotide bases, e.g., a second barcode sequence. Thus, with the predetermined barcode information (e.g., only the first and/or second barcode may be present in this pixel), the relative image intensity of the first barcode sequence and the second barcode sequence may be determined using: A*[0 1 1]+B*[00 1]=[0 1 3]; and A*[1 0 0]+B*[0 1 0]=[1 2 0], where A and B are numerical values. The value of A and B may determine the relative intensity of base calls from the first barcode sequence and the second barcode sequence. In this particular example, B is 2× of A indicating that the second barcode sequence contribute twice more to the image intensity of pixel (m,n) than the first barcode sequence. Such determination of linear combination of possible DNA sequence may be at a single pixel or multiple pixels. The same determination may be repeated at neighboring pixels or subpixels to confirm identical relative intensities. In response to determining neighboring pixels that may arrive at different relative intensity of base calls from the first barcode sequence and the second barcode sequence, weighting may be given to the pixels or subpixels that are closer to the center of the polonies at issue. In response to determining neighboring pixels that may arrive at different relative intensity of base calls from the first barcode sequence and the second barcode sequence, the decomposition of the intensity may be marked at failed.

In some embodiments, the obtained intensity from flow cell images may be rounded, e.g., to the nearest integer multiple of expected intensity value in that channel to discretize intensity. In some embodiments, before decomposition of at least partially overlapped base calls, base call sequences, e.g., barcodes, that are not possible in a location. i.e. are not “on” in a channel may be removed. In some embodiments, such filtering and removal of not-possible barcodes may be performed when the plexity of the data is greater than a predetermined threshold, e.g., 16, 20, 24, 30, or more.

In some embodiments, the set of equations for each channel may be solved separately, and all possible combinations of different base call sequences may be determined. Then the possible solutions from different channels may be combined to determine a solution that satisfies the sets equations for at least some or all the channels.

In some embodiments, image intensity quality metrics, such clarity, purity, chastity, quality score or the like may be used to identify pixel locations that may need decomposition operations to avoid decomposing all pixels include those with only a single base call.

The method 600 can comprise an operation 655 of performing 3D base calling using the extracted image intensities. Various existing 2D base calling algorithms can be used. The base calling results can be saved with its 3D location information, e.g., 3D coordinates. Such 3D coordinates can be used to register the base callings across different cycles and at different z locations, and/or to register the base callings to cell images herein.

In some embodiments, the operation 655 of performing base callings comprises performing primary analysis step(s) herein to adjust image intensities of polonies in the filtered images; and making base calls for the polonies based on the adjusted image intensities in the filtered images. The adjustment to image intensity in the filtered images can occur before or after filtering.

In some embodiments, the primary analysis steps comprise one or more of the following: background subtraction; image sharpening; intensity offset adjustment; color correction; intensity normalization; phasing and prephasing correction; quality score estimation.

In some embodiments, the methods 600, e.g., in FIGS. 6A-6B, comprise, before operation 610, providing a cellular sample harboring a plurality of RNA which comprises at least a first target RNA molecule and a second target RNA molecule.

In some embodiments, the methods 600, e.g., in FIGS. 6A-6B, comprise, before operation 610, generating a plurality of concatemer molecules including at least a first concatemer molecule that corresponds to a first target RNA molecule, and the plurality of concatemer molecules includes at least a second concatemer molecule that corresponds to a second target RNA molecule. In some embodiments, the operation of generating a plurality of concatemer molecules comprises one or more of: generating inside the sample a plurality of cDNA molecules which include at least a first target cDNA molecule that corresponds to the first target RNA molecule, and the plurality of cDNA molecules includes a second target cDNA molecule that corresponds to the second target RNA molecule; contacting the plurality of cDNA molecules in the sample with a plurality of target-specific padlock probes which includes at least a first plurality of target-specific padlock probes and a second plurality of target-specific padlock probes; closing the nick or gap in the at least first and second circularized target-specific padlock probes by conducting an enzymatic reaction, thereby generating at least a first covalently closed circular padlock probe and a second covalently closed circular padlock probe inside the sample; conducting a rolling circle amplification reaction inside the sample using the first and second covalently closed circular padlock probes as template molecules, thereby generating a plurality of concatemer molecules including at least a first concatemer molecule that corresponds to a first target RNA molecule, and the plurality of concatemer molecules includes at least a second concatemer molecule that corresponds to a second target RNA molecule; and sequencing the plurality of concatemer molecules inside the sample, which comprises sequencing the first concatemer molecule by conducting no more than 2 to 1000 sequencing cycles to generate a plurality of first sequencing read products, and sequencing the second concatemer molecule by conducting no more than 2 to 1000 sequencing cycles to generate a plurality of second sequencing read products.

In some embodiments, sequencing the plurality of concatemer molecules inside the sample comprises: contacting the plurality of concatemer molecules inside the sample with (i) a plurality of universal sequencing primers, (ii) a plurality of sequencing polymerases, and (iii) a plurality of nucleotide reagents, under a condition suitable for hybridizing the plurality of universal sequencing primers to their respective universal sequencing primer binding sites on the concatemers. The nucleotide reagents may comprise one or more of: multivalent molecules, nucleotides, and nucleotide analogs.

In some embodiments, sequencing the plurality of concatemer molecules inside the sample further comprises removing the plurality of first sequencing read products from the first concatemer molecules and retaining the first concatemer molecules in the sample, and removing the plurality of second sequencing read products from the second concatemer molecules and retaining the second concatemer molecules in the sample.

In some embodiments, the methods 600, e.g., in FIGS. 6A-6B, further comprises, before operation 610: providing the sample having a plurality of concatemer molecules immobilized on a support. Each concatemer molecule may correspond to a target RNA of a cellular sample. The support may be comprised in a flow cell disclosed herein.

In some embodiments, the operation 610 of obtaining the plurality of flow cell images of the sample comprises: generating, by the sequencing system, the plurality of flow cell images by conducting one or more cycles of sequencing reactions of the sample immobilized on the support. The plurality of flow cell images herein may be generated from at two or more different z levels along an axial axis from two or more color channels. The plurality of flow cell images herein may be generated from 3 to 1000 different z levels that can completed encompass the 3D volume of the 3D sample. The resolution along the z axis may be determined by the different number of z levels given the same 3D sample.

In some embodiments, the operation 610 of obtaining the plurality of flow cell images of the sample comprises: generating, by the sequencing system, the plurality of flow cell images by conducting one or more cycles of sequencing reactions of a plurality of concatemer molecules of the sample immobilized on the support. The sample herein may be a cellular sample. The sample may comprise polonies or clusters immobilized thereon. In some embodiments, the polonies corresponds to the plurality of nucleotide acid template molecules or concatemer molecules. In some embodiments, the operation of conducting the one or more cycles of the sequencing reactions comprises: contacting the plurality of concatemer molecules using a plurality of nucleotide reagents comprising a mixture of different types of nucleotide bases A, G, C and T/U. In some embodiments, the operation of conducting the one or more cycles of the sequencing reactions comprises: contacting the plurality of concatemer molecules with a plurality of sequencing primers, a plurality of polymerases, and a mixture of different types of avidites. The mixture of different types of avidites may include 2, 3, 4, or more types of avidites. The number of different types of avidites may match the number of different color channels of the sequencing system. The number of different types of avidites may be less than the number of different color channels of the sequencing system. An individual avidite in the mixture may comprise a core attached with multiple nucleotide arms and each arm of the individual avidite comprises the same type of nucleotide base. In some embodiments, the operation of conducting the one or more cycles of the sequencing reactions comprises: in each of the one or more cycles, imaging, by the optical system of the sequencing system, optical color signals emitted from nucleotide reagents that are bound to the plurality of concatemer molecules. In some embodiments, the operation of conducting the one or more cycles of the sequencing reactions comprises: in each of the one or more cycles, acquiring, by an optical system, the flow cell images comprising optical color signals emitted from nucleotide reagents that are bound to the plurality of concatemer molecules.

In some embodiments, the flow cell images herein may each comprise optical signals emitted from nucleotide reagents bound to a unbalanced diversity of nucleotide bases of A, G, C and T/U among the plurality of template or concatemer molecules immobilized on the support in one or more cycles.

In some embodiments, the plurality of polonies, e.g., corresponding to bright spots in the flow cell images, comprise a unbalanced diversity of nucleotide bases of A, G, C and T/U, and wherein the unbalanced diversity comprises a percentage of: (1) a number of one or more types of nucleotide bases within a region of the flow cell images to (2) a total number of nucleotide bases within the region, and the percentage is less than 20%, 15%, 10%, or 5% in one or more cycles. The region may include all of the FOV of the flow cell images. In some embodiments, the region may include only some of the FOV to separate areas within the FOV with spatial difference in nucleotide diversity.

### Cell Images and Staining

In some embodiments, the base callings are registered to one or more cell images herein. The one or more cell images may include images of the cell and/or tissue with one or more staining, e.g., fluorescent staining. In some embodiments, the one or more images can comprise staining of cellular structures that help locating polonies or clusters relative to the stained structures. For example, staining can be of cellular structures or components including but not limited to membranes, nuclei, and mitochondria. Different staining colors may be used to stain different components of the cell.

In some embodiments, the cell membrane after sequencing analysis and imaging using the sequencing system and reactions can be permeabilized. In some embodiments, the one or more cell images can comprise staining of lipids, such as lipids comprised in the cell membrane. In some embodiments, instead of labeling the lipids, the one or more cell images can comprise staining of one or more transmembrane proteins. The transmembrane proteins can be proteins embedded in the permeabilized membrane.

In some embodiments, the one or more cell images comprises fluorescence signals from cell membranes. The one or more cell images can be microscopic images. The one or more images can be fluorescent images. In some embodiments, different fluorescent colors can be included in the cell images. For example, the nuclei and the cell membrane can be stained with different colors.

In some embodiments, the one or more images can comprise segmentation of: cells, membranes, nuclei, or their combinations. FIG. 7B shows an exemplary cell image with segmentation of individual cells. In some embodiments, the edge(s) of each segment encompass the entire membrane of the cell within the segment. There can be only one cell in each segment. Some segments may not have any cell in them. In some embodiments, adjacent segments do not overlap with each other. In some embodiments, adjacent segments only overlap with each other by sharing one or more edges. In some embodiments, various segmentation algorithms can be used for segmenting the cells.

In some embodiments, the cell images disclosed herein are stained. The staining can occur after acquiring flow cell images using the sequencing system 110. In some embodiments, the staining can occur before acquiring sequencing images. The methods of staining the 3D sample such as the cells, tissue can include one or more operations disclosed herein. The staining of the 3D sample can use various methods that can specifically label one or more cell protein(s) that are located mostly in the membrane but with neglectable occurrence in other regions of the cell (e.g., less than 10%, 5%, 2% in amount or concentration).

In some embodiments, the cell images may be acquired using the sequencing system 100 herein without moving the sample(s) from its position during sequencing. It is advantageous to stain the sample after sequencing and acquire the cell images while keep the samples immobilized to the sample stage of the sequencing system. Some transformation, e.g., rotation, translation, shearing may still occur so that there is a need to registered the flow cell images during sequencing to the cell images acquired after sequencing and staining. In some embodiments, the cell images may be acquired using optical device(s) external to the sequencing system 100 after the sequencing run has been completed and after moving the sample away from the sequencing system 100.

The operations can include selecting one or more primary antibodies, each of the one or more primary antibody binding specifically to a corresponding protein. The corresponding protein can be a transmembrane protein of one or more cells. In some embodiments, the corresponding transmembrane protein does not exist in other cellular areas such as the cytosol or nuclei at a predetermined concentration so that staining of the transmembrane protein does not create perceivable signals in cellular areas other than the membrane. In some embodiments, one or more different transmembrane protein can be labeled by a primary antibody. For example, if there are 5 different types of transmembrane proteins, 5 different primary antibodies can be used and each primary antibody specifically binds to one of the transmembrane protein, but not the other transmembrane proteins. In some embodiments, a same type of primary antibody can non-specifically bind different proteins.

The staining methods can comprise an operation selecting one or more secondary antibodies that binds to the one or more primary antibodies. The staining methods can further comprise an operation of labeling the one or more secondary antibodies with a fluorescent label.

In some embodiments, the staining methods can further comprise an operation of linking the secondary antibody and the fluorescent label using a scaffold element or a tertiary probe, e.g., a hydrogel. The scaffold element can be used to retain mRNA of the membrane to facilitate binding and generation of fluorescent signal. In some embodiments, the mRNA can be any mRNAs in the cell. In some embodiments, the staining methods can comprise tissue clearing using various methods to remove some or all of parts of the cells to reduce the background fluorescence coming from portions of the cell that is not the membrane. In some embodiments, the fluorescent label comprises a fluorophore that re-emit light within a specific wavelength range upon light excitation. FIG. 8 shows an exemplary staining of the transmembrane protein using the staining method disclosed herein.

The staining methods can further comprise an operation of generating one or more cell images of the corresponding proteins. the one or more images contains fluorescent signal emitted from the fluorescent labels.

In some embodiments, the methods for base calling in sequencing data analysis can comprise generating a 3D polony map based on the plurality of filtered images. The 3D polony map can include a stack of 2D polony maps, each 2D polony map corresponding to a flow cell image acquired at the corresponding axial location. The 3D polony map can include some or all of polonies that can be identified in the axial stack of flow cell images.

In some embodiment, each 2D polony map can be generated based on the filtered image at the same axial location. In some embodiments, the 2D polony map can be generated from the filtered image similarly as the template image from flow cell images. In some embodiments, a 2D polony map is equivalent to a template image because both of them are virtual images that includes all the polonies identified in one or more cycles at a specific axial location.

As disclosed herein, the filtered image can advantageously exclude background objects that may interfere with signals from polonies. The filtered image can also remove some of the out-of-focus polonies or clusters. The parameters of the filtering, e.g., size and shape of the kernel, can be customized to balance between removing out-of-focus polonies and retaining relatively larger polonies or clusters that assemble out-of-focus polonies.

In some embodiments, each 2D polony map is registered to a template image (in 3D) or a reference coordinate system (in 3D). The template image or the reference coordinate system can be determined in a reference cycle. For each cycle different than the reference cycle, a 2D polony map can be generated and polony maps from different cycles can be registered relative to each other, using the template image or the reference coordinate system. In some embodiments, a single polony map can be generated for all channels with the same cycle. In some embodiments, the polony map is generated per channel per cycle.

In some embodiments, the 3D polony map is a volumetric polony map stacking all 2D polony maps at different axial locations. In some embodiments, the methods herein include removing duplicates of polonies from the stacked 2D polony maps to generate the 3D polony map. The 3D polony map without duplicates can be used as a reference to locate individual polonies in the sample for base calling. In some embodiments, the 3D polony map can be saved as a list of 3D coordinates that indicates the center of polonies.

In some embodiments, the methods herein further comprise an operation of extracting image intensity of polonies based on the 3D polony map. The image intensity can be extracted from one or more of: the flow cell images; the processed images; the filtered images. In some embodiments, image intensity can be extracted from filtered image. In some embodiments, the image intensity can be extracted from the filtered image after processing the filtered image with one or more primary analysis steps disclosed herein. For example, the filtered image can go through phasing and prephasing correction before the image intensity can be extracted.

In some embodiments, the 3D polony map can include duplicates of polonies, and such duplicates can be removed after base calling. For example, all the polonies in the 3D polony map including the duplicates can be used to extract image intensities for base calling. Candidate duplicates of polonies can be identified as polonies at different z locations, e.g., adjacent z locations, and at same x,y locations. If the base calling of such candidate duplicates are identical, one of them can be removed as duplicates. Alternatively, both of them can be removed, and a new polony representing both can be added at the z level as an average of the two and at the same x and y locations.

In some embodiments, the methods herein further comprise an operation of performing 3D base callings based on the extracted image intensity of the polonies. Various 2D base calling algorithms can be used here. For example, base calling of a polony can be made by comparing image intensity of the same polony from different channels, and call the base that corresponds to the largest image intensities among all the channels.

In some embodiments, the operation of filtering flow cell images thereby generating a plurality of filtered images can include performing deconvolution of the plurality of flow cell images. The deconvolution can be at least along the axial direction. In some embodiments, the deconvolution can be 3D. The deconvolution can be in the spatial domain or performing its equivalent in a transformed domain, e.g., the Fourier domain. The deconvolution is configured to reduce or remove the spreading or blurring effect of the optical system on the polonies so that the size and shape of the polonies can appear more accurate in flow cell images. In some embodiments, the deconvolution operation can be used alone as the filtering operation or in combination with other filtering operations, such as the top hat filtering.

### Image Registration to Cell Images

Various methods can be used for registering flow cell images based on fiducial markers. The fiducial markers can be internal or external to the sample. For example, internal fiducial markers can include at least some of the polonies or clusters or background objects in the sample. As another example, external fiducial markers can be microspheres coated on the flow cell so that the signal from the microspheres can function similarly as internal fiducial markers for registration. The same fiducial markers can appear in sequencing images, e.g., the MIP image(s), the flow cell image(s), the filtered images, as well as the cell images so that transformation(s) can be derived from aligning the fiducial markers in different images. The transformation(s) can be used for registering or aligning the sequencing image(s) and cell image(s) and objects that appear in them. Exemplary embodiments of image registration methods are described in PCT patent application No. PCT/US2023/067931 (where the contents of the patent are hereby incorporated by reference in its entirety).

For example, a polony or other object, e.g., background objects, with image intensity I centers at location (x1,y1) in a sequencing image can appear at location (x2, y2) with intensity I′ in a cell image, where (x2,y2) ≃Mr*(x1,y1), and Mr is the transformation matrix. Similarly, the inverse transformation matrix Mr⁻¹ can be determined such that (x1,y1)≃Mr⁻¹*(x2,y2). The registration of images can be in 2D and can include translation, scaling, rotation, and/or shearing of flow cell images among different channels. Multiple points in the sequencing image and their corresponding points in the cell image can be used to determine the transformation. The minimum number of points that is needed can be determined by the degree of freedom in the transformation. In some embodiments, the image registration can be 3D with coordinates in x, y, and z axes.

In some embodiments, a sequencing image can be divided into multiple subtitles, and a transformation can be determined for each subtile to represent the transformation of the whole image. In some embodiments, the image transformation of each subtile can be uniquely represented by a transformation matrix. The transformation matrix can be determined as below:

$$( \begin{matrix}a 1 & a 2 & & an \\ b 1 & b 2 & ... & bn \\ 1 & 1 & & 1\end{matrix} ) ≃ ( \begin{matrix}M 11 & M 12 & M 13 \\ M 21 & M 22 & M 23 \\ M 31 & M 32 & M 33\end{matrix} ) ( \begin{matrix}x 1 & x 2 & & xn \\ y 1 & y 2 & ... & yn \\ 1 & 1 & & 1\end{matrix} ) \qquad ( 1 )$$

$$M = ( \begin{matrix}M 11 & M 12 & M 13 \\ M 21 & M 22 & M 23 \\ M 31 & M 32 & M 33\end{matrix} ) \qquad ( 2 )$$

where n is the number of subtiles, a1=x1+dx1, b1=y1+dy1, a2=x2+dx2, b2=y2+dy2, . . . an=xn+dxn, bn=yn+dyn, d1 . . . dn are 2D shifts corresponding to the subtiles, and where dxn and dyn are shift components of the 2D shift, dn, in the x and y axis, respectively, and wherein M is the 3×3 transformation matrix of the subtile.

In some embodiments, the transformation matrix can be defined as the inverse matrix of M, i.e., M⁻¹, so that equation (1) can be expressed differently as

$${M}^{- 1} ( \begin{matrix}a 1 & a 2 & & an \\ b 1 & b 2 & ... & bn \\ 1 & 1 & & 1\end{matrix} ) ≃ ( \begin{matrix}x 1 & x 2 & & xn \\ y 1 & y 2 & ... & yn \\ 1 & 1 & & 1\end{matrix} ) \qquad ( 3 )$$

In some embodiments, the transformation matrix M is an estimation in equations (1) and (3) based on the 2D shifts. In some embodiments, the value of n may affect the accuracy of the estimation.

In some embodiments, more than one region can be selected within a subtile for cross correlation calculation, and more than one 2D shift can be calculated for each subtile and used for estimating the transformation of the subtile. In these embodiments, n in equation (1) can be replaced by a larger number, e.g., 2*n when 2 regions are selected per subtile, and the transformation matrix M can be estimated using equations (1) and (2).

In some embodiments, (a1,b1) . . . (an, bn) in equations (1)-(3) are coordinates for selected region(s) (e.g., coordinates of a center pixel of the corresponding region(s)) after transformation, (x1, y1) . . . (xn, yn) are coordinates of the selected region(s) before transformation, e.g., coordinates of a center pixel.

In some embodiments, n is a number that is no less than 3. The larger the n, the more information can be used to estimate the transformation matrix M. In some embodiments, n is not greater than 9.

In some embodiments, the transformation of one or more subtiles is linear. In some embodiments, the transformation of all subtiles is linear. In some embodiments, the transformation matrix is a matrix in which M31 and M32 is equal to 0, and M33 is 1. In some embodiments, one or more of the transformations per subtile is an affine transformation and the transformation matrix of the entire flow cell image is an affine matrix.

In some embodiments, the transformation matrix M is an estimation in equations (1) and (3) based on the size of the selected region(s). In some embodiments, the size of selected region may affect the accuracy of the estimation. In some embodiments, the size of the select region can be about 128×128. In some embodiments, the size of the selected region can be about 32×32, 48×48, 64×64, 96×96, 160×160, 196×196, 256×256, or of various different sizes. The transformations per subtile as disclosed herein can be calculated using a selected region within a subtile, the selected region can be equal to or smaller than the subtile. In either case, the transformation estimated using the region can be used to estimate the transformation of the entire subtile given the intrinsic characteristics of image transformation across sequencing cycles. The image transformation between cycles and/or between neighboring pixels can be relatively small, e.g., with less than about 8%, 5% or less than about 1% of scaling, rotation, and/or shearing. In some embodiments, the transformations disclosed herein can include an image translation with greater than about 5% difference between cycles and/or between neighboring pixels.

After the plurality of transformations are determined for individual subtiles, the transformation of entire the flow cell image can be accurately and reliably estimated by transforming individual subtiles using the plurality of transformations and combining the transformed subtiles into a transformed flow cell. The techniques disclosed herein advantageously estimate the transformation of the flow cell image by determining a plurality of transformations of its individual subtiles. The plurality of transformations can be linear and yet accurately and reliably estimate the transformation of the flow cell image even if the transformation is non-linear. The techniques disclosed herein advantageously eliminate the need to calculate the transformation of the entire flow cell image which can be more computationally intensive and time-consuming and prone to failure than estimating a plurality of transformations for the subtiles.

### Image Registration Across Channels and Cycles

In some embodiments, the method include an operation of aligning or registering the flow cell images across different sequencing cycles, from different channels, and/or at different z levels to a common coordinate system before base calling. The common coordinate system can be the reference coordinate system disclosed herein. The common coordinate system can be predetermined. The common coordinate system can be the reference coordinate system disclosed herein. The common coordinate system can be predetermined. The common coordinate system may be a Cartesian coordinate system. Various other coordinate systems may be used. Other coordinate systems can include but are not limited to the polar coordinate system, cylindrical, or spherical coordinate systems. Exemplary embodiments of image registration methods are described in PCT patent application No. PCT/US2023/067931 (where the contents of the patent are hereby incorporated by reference in its entirety).

Prior to registering to the cell images, the flow cell images can be registered relative to each other so that polonies or clusters in different cycles and/or channels can be aligned and base calling can be accurate and reliable respect to specific polonies or clusters.

Various methods can be used to register the sequencing images, e.g., flow cell images, filtered images, or MIP images, of different cycles and/or channels.

In some embodiments, the methods 600 include an operation of registering the first and/or second MIP images, e.g., 900 in FIG. 9. In some embodiments, the MIP images are registered across channels and different cycles before any base calling are performed. The MIP images can be registered using 2D registration techniques for example, by treating the MIP images as flow cell images acquired from the sequencing system 110. In some embodiments, the MIP images can be registered, e.g., across different channels and/or different cycles, using the image registration method 900 as disclosed herein by treating each MIP image as a flow cell image. In some embodiments, the MIP images can be registered after one or more preprocessing operations disclosed herein are performed.

For example, the flow cell images can be registered to the reference coordinate system that is common to all the flow cell images so that flow cell images from different cycles and/or channels can be aligned relative to each other. The reference coordinate system can be determined in the reference cycle or any other predetermined cycle. For example, a reference coordinate system can be the coordinate system of the flow cell image from one channel. As another example, the reference coordinate system can be based on the external fiducial markers or other objects external to the flow cell images.

In some embodiments, the method 600 includes an operation of generating one or more template images in the reference coordinate system by registering the polonies to the one or more template images using the coordinates thereof. FIG. 8A shows a schematic diagram of one or more template images generated in the reference cycle in a reference coordinate system. The template image 210, in some embodiments, include a size that is about identical to a single tile 210 that includes a 5×5 grid of subtiles 220. A region 230 is selected in each subtile and includes center pixels of the corresponding subtile. The reference coordinate system in this embodiment has an origin 212 at its top left pixel. In some embodiments, the template image disclosed herein can be individual regions such as region 230. Each template image can include a plurality of polonies 232 therein.

In some embodiments, the template image can be of about the same size of a flow cell image so that all the polonies, from different tiles, 210 in FIGS. 8A-8B, and from multiple channels, can be registered to the same template image. However, such template image may contain polonies that will not be used in at least some operations described herein to reduce computational burden without sacrificing accuracy.

In some embodiments, more than one template images can be generated, and each template image 230 corresponds to at least part of a subtile of a flow cell image from a channel.

The template image herein can be initialized as a virtual image that has a black or dark background with no signals from polonies. For example, the template image can be initialized to be zero or include otherwise minimal image intensity at all pixels.

After the coordinates of a polony is determined in operation 910 by image registration of flow cell images across different channels, the intensity of the polony can be added to the template image at the location determined by the coordinates and with the size and shape determined based on registration. The template image can be a virtual image that combines image intensity from polonies obtained from 2, 3, 4, or even more channels at the reference cycle. The pixels of the template containing no polonies in them remains to be black or dark so that the template image can have a cleaner background without noise that appear in actual flow cell images.

In some embodiments, the method 900 includes an operation of obtaining image intensities, sizes, shapes, or their combinations of the polonies from at least a portion of one or more subtiles in the reference cycle so that such information can be used to include the polonies in the template image. In some embodiments, polonies can have a fixed shape and/or size. In some embodiments, a point spread function determined by the optical system herein is used to determine the fixed shape and/or size of polonies. In some embodiments, the polonies has a fixed spot size that is based on the sigma of a Gaussian point spread function. In some embodiments, one or more polonies have a size of 1-9 pixels. In some embodiments, one or more polonies have a size of 1-3 pixels.

The template image can include polonies from different channels along with the channel information. As an example, the channel information can be provided as a label or a specific order of how the polonies are included.

In some embodiments with multiple template images, each template image 230 can cover a region within a subtile, and such template image may but is not required to include all the polonies within the subtile.

In some embodiments, the method 900 includes an operation 930 of obtaining a flow cell image in a cycle after the reference cycle. The operation 930 can include passively receiving or actively requesting the flow cell image from an optical system disclosed herein after the flow cell image is generated by the optical system. The optical system can be included in the imager 116 in FIG. 1.

The flow cell image can include some or all of the same polonies in the template image(s) of the reference cycle. In particular, the flow cell image can include some or all of the same polonies in regions corresponding to the selected region in the reference cycle.

FIG. 8A shows the flow cell image acquired in a cycle different from the reference cycle at the bottom. The flow cell image 240 is acquired with multiple subtiles 250. The selected region 260 in this cycle can be at the same location relative to the new origin 242 in this cycle as the selected region 230 to the origin 212 in the reference cycle. In this cycle, the flow cell image 210 in the reference may have transformed to the transformed image 211, and the selected region 230 correspondingly transformed to region 231 with some overlap to region 260. The image transformation herein can be 2D, and can include translation, scaling, rotation, and/or shearing.

In some embodiments, the method 900 is configured to align template image 210 or 230 in the reference cycle and the transformed image 211 or 231 in another cycle to the reference coordinate system.

In some embodiments, instead of using region 231 or 211 directly in image registration, the method 900 can include an operation of selecting region 230 and 260 for simpler and more convenient determination of image registration. The region 260 can include at least part of the polonies 232 that were in the template image 230.

In some embodiments, the method 900 comprises an operation 940 of determining a plurality of transformations of the flow cell image 240 based on the one or more template images 210 or 230. As shown in FIG. 8A, each of the plurality of transformations can correspond to a subtile 250 of the flow cell image 240 and is configured to register the subtile 250 of the flow cell 240 image to a corresponding portion of the template image 210 (if the template image includes the entire tile) or a corresponding template image 230 (if there are multiple template images within the tile).

In some embodiments, the operation 540 may include determining each transformation corresponding to a subtile of the flow cell image. More particularly, each transformation can correspond to a selected region in each of some or all the subtiles. A region can be selected in various ways from a subtile to include at least part of the subtile. The region may be a predetermined two-dimensional shape, e.g., rectangle, circle, or square. As a nonlimiting example, the selected region can include one or more center pixels of the subtile as shown in FIG. 8A at 260. The size of the region can be determined to balance the trade-off between computational complexity and accuracy of image registration. For example, selecting a 64×64 region can be computationally simpler than selecting a 128×128 region but may not be as accurate. In some embodiments, the selected region includes some or all of the polonies 232 registered in the template image(s) in the reference cycle so that the same polonies and their relative locations in the template image(s) and the flow cell image can be used for determining the transformation. In some embodiments, the size of the template image, e.g., 230 and the region 260 can be identical or about identical. In some embodiments, the size of the template image 210 or 220 and the selected region 260 can be different.

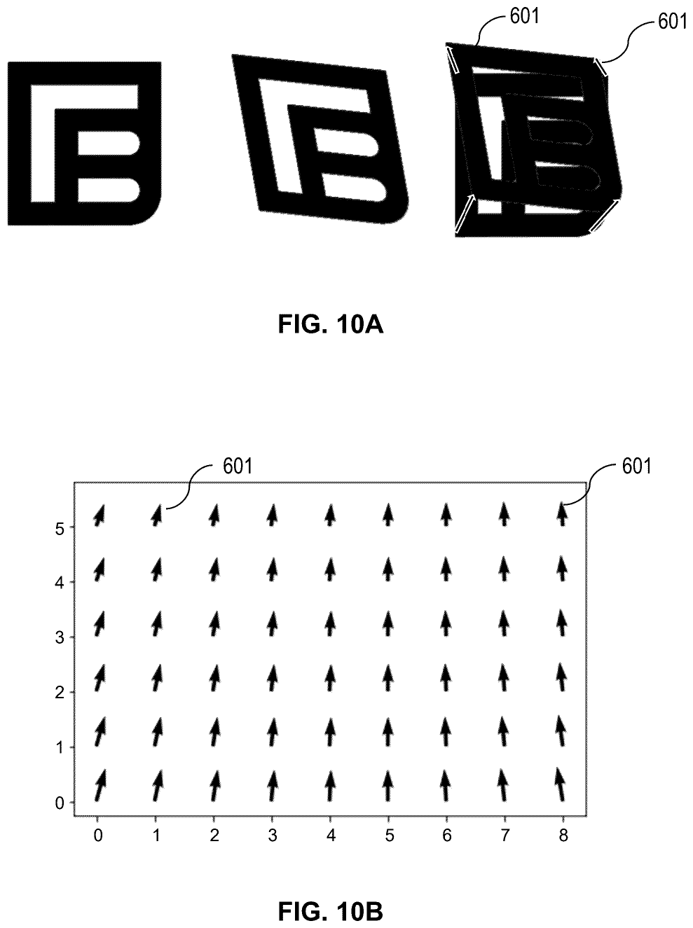

In some embodiments, cross correlation of the selected region and the template image can be computed for determining a 2D shift of the region relative to the template image. FIG. 10A shows a reference image (left) that is transformed with 2D shear, scaling, and rotation into a different image (middle). 2D shifts 601 at the four corners of the reference image can be determined, for example, using the methods disclosed herein using cross correlation. And the 2D shifts at four corners can be used to estimate the transformation between the two images.

In some embodiments, cross correlation can be calculated in the spatial domain. In some embodiments, cross correlation can be calculated in the spatial frequency domain after Fourier transform (FT). The method 900 may comprise generating a corresponding Fourier Transformed Image (FTI) of a template image and a Fourier transform of the selected region. The Fourier transformation herein can be calculated using discrete FT (DFT), fast FT (FFT), or the like. The cross correlation can be determined based on the FTI and the Fourier transform of the selected region. As a nonlimiting example, the cross correlation can be the elementwise multiplication of the FTI with the FT of the selected region, with a complex conjugate or rotation of the one of them. Then, an inverse FT of the elementwise multiplication can be obtained. In some embodiments, the cross correlation can be a 2D image with a peak intensity at its coordinate [xp, yp]. In some embodiments, a 2D shift can be determined based the coordinates [xp, yp] in comparison to the coordinates of a peak obtained from cross-correlation of two original images without transformation. The 2D shift of the selected region 260 can be used to estimate the 2D shift for the entire subtile. In some embodiments, results of calculating the cross correlation in the spatial domain or Fourier domain can be equivalent. In some embodiments, calculation in the Fourier domain can be simpler and more efficient than calculation in the spatial domain.

In some embodiments, the image transformation of the subtile can be determined from 2D shifts from some or all neighboring subtiles with or without the 2D shift from itself. In some embodiments, 2D shifts from all immediate neighbors can be used. For example, to determining transformation of subtile 253, 2D shifts from 3 neighboring subtiles and the 2D shift from itself can be used. For subtile 251, a total number of 6 2D shifts including immediate neighboring subtiles and themselves can be used. For subtile 252, a total number of 9 2D shifts including neighboring subtiles and itself can be used. In some embodiments, 2D shifts from some but not all neighboring subtiles can be used. In some embodiments, 2D shifts from all neighboring subtiles except 1-2 outliers can be used to determine the transformation. The outlier(s) can be excluded using a predetermined criterium, e.g., more than 30% or 50% different from other 2D shifts.

FIG. 10B is an image showing 2D shifts within a tile of a flow cell image. In this embodiment, the tile has a 6×9 grid of subtiles, and each subtile has a 2D shift 601 that is determined using the technologies disclosed herein. Each shift has a magnitude of less than about 5 pixels along x or y axis. A pixel size may vary depending on imaging parameters, an exemplary pixel can be from 0.01 um to 0.9 um. The 2D shifts 601 can be used to calculate transformation, e.g., affine matrix, for the tile, by individually calculating a transformation for each subtile. In this embodiment, an affine matrix can be calculated using the methods disclosed herein.

In some embodiments, subpixel resolution, e.g., about 0.01, 0.02, 0.03, or 0.05 pixel, of the 2D shifts 601 can be achieved using various methods including interpolation, upsampling, etc. In some embodiments, subpixel resolution can be achieved by fitting the peak with a selected filter, e.g., a 3×3 or 5×5 Gaussian filter.

In some embodiments, the image transformation of a subtile can be uniquely represented by a transformation matrix. The transformation matrix can be determined as below:

$$( \begin{matrix}a 1 & a 2 & & an \\ b 1 & b 2 & ... & bn \\ 1 & 1 & & 1\end{matrix} ) ≃ ( \begin{matrix}M 11 & M 12 & M 13 \\ M 21 & M 22 & M 23 \\ M 31 & M 32 & M 33\end{matrix} ) ( \begin{matrix}x 1 & x 2 & & xn \\ y 1 & y 2 & ... & yn \\ 1 & 1 & & 1\end{matrix} ) \qquad ( 4 )$$

$$M = ( \begin{matrix}M 11 & M 12 & M 13 \\ M 21 & M 22 & M 23 \\ M 31 & M 32 & M 33\end{matrix} ) \qquad ( 5 )$$

where n is the number of subtiles, a1=x1+dx1, b1=y1+dy1, a2=x2+dx2, b2=y2+dy2, . . . an=xn+dxn, bn=yn+dyn, d1 . . . dn are 2D shifts corresponding to the subtiles, and where dxn and dyn are shift components of the 2D shift, dn, in the x and y axis, respectively, and wherein M is the 3×3 transformation matrix of the subtile.

In some embodiments, the transformation matrix can be defined as the inverse matrix of M, i.e., M⁻¹, so that equation (1) can be expressed differently as

$${M}^{- 1} ( \begin{matrix}a 1 & a 2 & & an \\ b 1 & b 2 & ... & bn \\ 1 & 1 & & 1\end{matrix} ) ≃ ( \begin{matrix}x 1 & x 2 & & xn \\ y 1 & y 2 & ... & yn \\ 1 & 1 & & 1\end{matrix} ) \qquad ( 6 )$$

In some embodiments, the transformation matrix M is an estimation in equations (4) and (6) based on the 2D shifts. In some embodiments, the value of n may affect the accuracy of the estimation. In some embodiments, more than one region can be selected within a subtile for cross correlation calculation, and more than one 2D shift can be calculated for each subtile and used for estimating the transformation of the subtile. In these embodiments, n in equation (1) can be replaced by a larger number, e.g., 2*n when 2 regions are selected per subtile, and the transformation matrix M can be estimated using equations (4) and (5).

In some embodiments, (a1,b1) . . . (an, bn) in equations (1)-(3) are coordinates for selected region(s) (e.g., coordinates of a center pixel of the corresponding region(s)) after transformation, (x1, y1) . . . (xn, yn) are coordinates of the selected region(s) before transformation, e.g., coordinates of a center pixel.

In some embodiments, n is a number that is no less than 3. The larger the n, the more information can be used to estimate the transformation matrix M. In some embodiments, n is not greater than 9.

In some embodiments, the transformation of one or more subtiles is linear. In some embodiments, the transformation of all subtiles is linear. In some embodiments, the transformation matrix is a matrix in which M31 and M32 is equal to 0, and M33 is 1. In some embodiments, one or more of the transformations per subtile is an affine transformation and the transformation matrix is an affine matrix.

In some embodiments, the transformation matrix M is an estimation in equations (4) and (6) based on the size of the selected region(s). In some embodiments, the size of selected region may affect the accuracy of the estimation. In some embodiments, the size of the select region can be about 128×128. In some embodiments, the size of the selected region can be about 32×32, 48×48, 64×64, 96×96, 160×160, 196×196, or 256×256. The transformations per subtile as disclosed herein can be calculated using a selected region within a subtile, the selected region can be equal to or smaller than the subtile. In either case, the transformation estimated using the region can be used to estimate the transformation of the entire subtile given the intrinsic characteristics of image transformation across sequencing cycles. The image transformation between cycles and/or between neighboring pixels can be relatively small, e.g., with less than about 5% or less than about 1% of scaling, rotation, and/or shearing. In some embodiments, the transformations disclosed herein can include an image translation with greater than about 5% difference between cycles and/or between neighboring pixels.

After the plurality of transformations are determined for individual subtiles, the transformation of the flow cell image can be accurately and reliably estimated by the plurality of transformations. The techniques disclosed herein advantageously estimate the transformation of the flow cell image by determining a plurality of transformations of its individual subtiles. The plurality of transformations can be linear and yet accurately and reliably estimate the transformation of the flow cell image even if the transformation is non-linear. The techniques disclosed herein advantageously eliminate the need to calculate the transformation of the entire flow cell image which can be more computationally intensive and time-consuming than estimating a plurality of transformations for the subtiles.

In some embodiments, the computer-implemented method 900 further include an operation of saving the plurality of transformation by the processor disclosed herein. In some embodiments, the computer-implemented method 900 further include an operation of communicating the plurality of transformations to a processing unit such as a CPU for subsequent operations.

In some embodiments, the computer-implemented method 900 further include registering subtitles to the one or more template images using the plurality of transformations. This operation can be performed by the processing unit such as the CPU(s). In any given cycle different from the reference cycle, each subtile can be registered or transformed to the one or more template images by multiplying the subtile by the transformation matrix corresponding to the subtile.

In some embodiments, the computer-implemented method 900 may include an operation of performing one or more preprocessing steps on the flow cell images of the reference cycle and/or other cycles before registration of images from that cycle.

In some embodiments, this operation of performing one or more preprocessing steps can be performed by the FPGA(s) or NPU(s). In some embodiments, the data after the operation can be communicated by the FPGA(s) or NPU(s) to the CPU(s) so that CPU(s) can perform subsequent operation(s) in method 600 900 using such data.

In some embodiments, the one or more preprocessing steps of flow cell images in the reference cycle can be performed before operation 910, 920 or after 920. In some embodiments, the one or more preprocessing steps of flow cell images in the reference cycle can be performed after the operation of receiving the flow cell images in the reference cycle from the optical system disclosed herein. In some embodiments, the one or more preprocessing steps of flow cell images in the reference cycle can be performed before the operation of obtaining image intensities, sizes, shapes, or their combinations of the polonies from the plurality of subtiles of the flow cell images in the reference cycle.

In some embodiments, the one or more preprocessing steps of flow cell images in cycles other than the reference cycle can be performed after operation 930 or 940. In some embodiments, the one or more preprocessing steps of flow cell images in cycles other than the reference cycle can be performed after the operation of registering the subtiles of flow cell image to the one or more template images. In some embodiments, the one or more preprocessing steps of flow cell images in cycles other than the reference cycle can be before the operation of extracting image intensities of a plurality of polonies from the subtiles of the flow cell image. In some embodiments, the one or more preprocessing steps of flow cell images in cycles other than the reference cycle can be before the operation of making base calls using image intensities of the subtiles of the flow cell image.

The one or more preprocessing steps can comprise background subtraction. The background subtraction is configured to remove at least some background signal that may interfere with the signal of interest, i.e., image intensities of the polonies. The background signal can be noise caused by multiple sources including the flow cell 112, the imager 115, the sequencer 114, and other sources. The background subtraction can be adjusted to avoid over subtraction.

The one or more preprocessing steps can include image sharpening so that image intensities of polonies can be optimized in consideration of their surroundings in the flow cell images. For example, a Laplacian of Gaussian (LoG) filter can be used for sharpening.

The one or more preprocessing steps can include image registration so that image intensities of polonies can be registered relative to each other. For example, the image intensities can be registered to the template as disclosed herein.

The one or more preprocessing steps can include intensity offset adjustment that can remove the offset in the intensity that has not been removed during background subtraction.

The one or more preprocessing steps can include color correction to remove interference of one channel from other channels or colors.

The one or more preprocessing steps can include phasing and prephasing correction which is configured to correct image intensities within a specific cycle by removing intensity biases caused by sequencing of DNA fragments that are out of synchronization from other fragments by either falling behind or getting ahead.

The one or more preprocessing steps can include intensity normalization so that the image intensity of polonies from different channels can be normalized to be within a predetermined range.

The one or more preprocessing steps can comprise: background subtraction; image sharpening; or a combination thereof.

In some embodiments, the computer-implemented method 900 further include extracting image intensities of a plurality of polonies, from the subtiles registered to the template image(s). This operation can be performed by the processing unit such as the CPU(s) or FPGA(s). In some embodiments, polonies with their corresponding intensities are extracted from the flow cell image(s) into a different data format that is simpler and more efficient to handle. For example, each polony can have 4 different intensities, each intensity from a different channel. Such intensities can be extracted into a list, with each entry of the list corresponding to a polony. The list can be generated after image registration to reflect location information of the same polonies in different cycles. As such, image intensities of the same polony in different cycles can be located in different lists each corresponding to a cycle.

In some embodiments, the computer-implemented method 900 further include making base calls using image intensities of the subtiles of the flow cell image after the registration so that base calling can be made accurately relative to the same polonies across different channels and in different cycles.

In some embodiments, the method 900 include an operation 940 of determining a plurality of transformations of the flow cell image. The operation 940 can include determining each of the transformations without using any neighboring subtiles as disclosed herein. Instead, more than 2 regions can be selected within the subtile, and 2D shift can be determined for each of the regions. The transformation of the subtile can be determined using the 2D shifts obtained from regions within the same subtile using equations (1) and (2). The regions within a subtile can be smaller in size than the region 260 in neighboring subtiles. For example, the region 260 can be about 128×128, and the regions within a subtile can be 3, 4, 5, or even more regions, and each region include a about 64×64 matrix. The other operations of method 900 can remain the same for image registration with or without using neighboring subtiles in generating the transformations.

### Optical Systems

The imager 116 in FIG. 1 can include one or more optical systems. Further disclosed herein are optical system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus enable higher throughput image acquisition and analysis.

In some instances, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being images.

In some instances, improvements in imaging performance, e.g., for dual-side (flow cell) imaging applications comprising the use of thick flow cell walls (e.g., wall (or coverslip) thickness >700 μm) and fluid channels (e.g., fluid channel height or thickness of 50-200 μm) may be achieved even when using commercially—available, off-the-shelf objectives by using a tube lens design that corrects for the optical aberrations induced by the thick flow cell walls and/or intervening fluid layer in combination with the objective.

In some instances, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel.

Exemplary embodiments disclosed herein may comprise fluorescence imaging systems, said systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 μm, at least 500 μm, at least 600 μm, at least 700 μm, at least 800 μm, at least 900 μm, at least 1000 μm, or a working distance falling within a range defined by any two of the foregoing; and wherein the field-of-view has an area of at least 0.1 mm², at least 0.2 mm², at least 0.5 mm², at least 0.7 mm², at least 1 mm², at least 2 mm², at least 3 mm², at least 5 mm², or at least 10 mm², or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some embodiments, the numerical aperture may be at least 0.75. In some embodiments, the numerical aperture is at least 1.0. In some embodiments, the working distance is at least 850 μm. In some embodiments, the working distance is at least 1,000 μm. In some embodiments, the field-of-view may have an area of at least 2.5 mm². In some embodiments, the field-of-view may have an area of at least 3 mm². In some embodiments, the spatial sampling frequency may be at least 2.5 times the optical resolution of the fluorescence imaging system. In some embodiments, the spatial sampling frequency may be at least 3 times the optical resolution of the fluorescence imaging system. In some embodiments, the system may further comprise an X-Y-Z translation stage such that the system is configured to acquire a series of two or more fluorescence images in an automated fashion, wherein each image of the series is or can be acquired for a different field-of-view. In some embodiments, a position of the sample plane may be simultaneously adjusted in an X direction, a Y direction, and a Z direction to match the position of an objective lens focal plane in between acquiring images for different fields-of-view. In some embodiments, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some embodiments, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some embodiments, the specified error threshold is 100 nm or greater. In some embodiments, the specified error threshold is 50 nm or less. In some embodiments, the system comprises three or more image sensors, and wherein the system is configured to image fluorescence in each of three or more wavelength ranges onto a different image sensor. In some embodiments, a difference in the position of a focal plane for each of the three or more image sensors and the sample plane is less than 100 nm. In some embodiments, a difference in the position of a focal plane for each of the three or more image sensors and the sample plane is less than 50 nm. In some embodiments, the total time required to reposition the sample plane, adjust focus if necessary, and acquire an image is less than 0.4 seconds per field-of-view. In some embodiments, the total time required to reposition the sample plane, adjust focus if necessary, and acquire an image is less than 0.3 seconds per field-of-view.

Also disclosed herein are fluorescence imaging systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some embodiments, the objective lens may be a commercially-available microscope objective. In some embodiments, the commercially-available microscope objective may have a numerical aperture of at least 0.3. In some embodiments, the objective lens may have a working distance of at least 700 μm. In some embodiments, the objective lens may be corrected to compensate for a cover slip thickness (or flow cell wall thickness) of 0.17 mm or of greater or lesser thickness than 0.17 mm. In some embodiments, the optical system may be corrected to compensate for cover slip thickness, flow cell thickness, or distance between desired focal planes. In some embodiments, said correction may be made by inserting a corrective optic, such as a lens or optical assembly into the light path of the optical system. In some embodiments, said correction may be made without inserting a corrective optic, such as a lens or optical assembly into the light path of the optical system. In some embodiments, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some embodiments, the at least one tube lens may be a compound lens comprising three or more optical components. In some embodiments, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some embodiments, the at least one tube lens is configured to correct an imaging performance metric for a combination of the objective lens, the at least one tube lens, and the at least one image sensor when imaging an interior surface of a flow cell having a wall thickness of at least 1 mm. In some embodiments, the at least one tube lens is configured to correct an imaging performance metric for a combination of the objective lens, the at least one tube lens, and the at least one image sensor when imaging an interior surface of a flow cell having a gap of at least 100 μm. In some embodiments, the at least one tube lens is configured to correct an imaging performance metric for a combination of the objective lens, the at least one tube lens, and the at least one image sensor when imaging an interior surface of a flow cell having a gap of at least 200 μm. In some embodiments, the system comprises a single objective lens, two tube lenses, and two image sensors, and each of the two tube lenses is designed to provide optimal imaging performance at a different fluorescence wavelength. In some embodiments, the system comprises a single objective lens, three tube lenses, and three image sensors, and each of the three tube lenses is designed to provide optimal imaging performance at a different fluorescence wavelength. In some embodiments, the system comprises a single objective lens, four tube lenses, and four image sensors, and each of the four tube lenses is designed to provide optimal imaging performance at a different fluorescence wavelength. In some embodiments, the design of the objective lens or the at least one tube lens is configured to optimize the modulation transfer function in the mid to high spatial frequency range. In some embodiments, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some embodiments, the difference in the imaging performance metric for imaging the upper interior surface and the lower interior surface of the flow cell is less than 10%. In some embodiments, the difference in imaging performance metric for imaging the upper interior surface and the lower interior surface of the flow cell is less than 5%. In some embodiments, the use of the at least one tube lens provides for an at least equivalent or better improvement in the imaging performance metric for dual-side imaging compared to that for a conventional system comprising an objective lens, a motion-actuated compensator, and an image sensor. In some embodiments, the use of the at least one tube lens provides for an at least 10% improvement in the imaging performance metric for dual-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some embodiments, the illumination system further comprises a condenser lens. In some embodiments, the specified field-of-illumination has an area of at least 2 mm2. In some embodiments, the light delivered to the specified field-of-illumination is of uniform intensity across a specified field-of-view for an imaging system used to acquire images of the support surface. In some embodiments, the specified field-of-view has an area of at least 2 mm2. In some embodiments, the light delivered to the specified field-of-illumination is of uniform intensity across the specified field-of-view when a coefficient of variation (CV) for light intensity is less than 10%. In some embodiments, the light delivered to the specified field-of-illumination is of uniform intensity across the specified field-of-view when a coefficient of variation (CV) for light intensity is less than 5%. In some embodiments, the light delivered to the specified field-of-illumination has a speckle contrast value of less than 0.1. In some embodiments, the light delivered to the specified field-of-illumination has a speckle contrast value of less than 0.05.

### Imaging Modules and Systems

It will be understood by those of skill in the art that the disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectic focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

The present disclosure provides methods for sequencing immobilized or non-immobilized template molecules. The methods can be operated in system 100, for example, in sequencer 114. In some embodiments, the immobilized template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized template molecules comprise a plurality of nucleic acid template molecules each having two or more tandem copies of a target sequence of interest (e.g., concatemers). In some embodiments, nucleic acid template molecules comprising concatemer molecules can be generated by conducting rolling circle amplification of circularized linear library molecules. In some embodiments, the non-immobilized template molecules comprise circular molecules. In some embodiments, methods for sequencing employ soluble (e.g., non-immobilized) sequencing polymerases or sequencing polymerases that are immobilized to a support.

In some embodiments, the sequencing reactions employ detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs. In some embodiments, the sequencing reactions employ phosphate chain labeled nucleotides.

### Linear Library Molecules

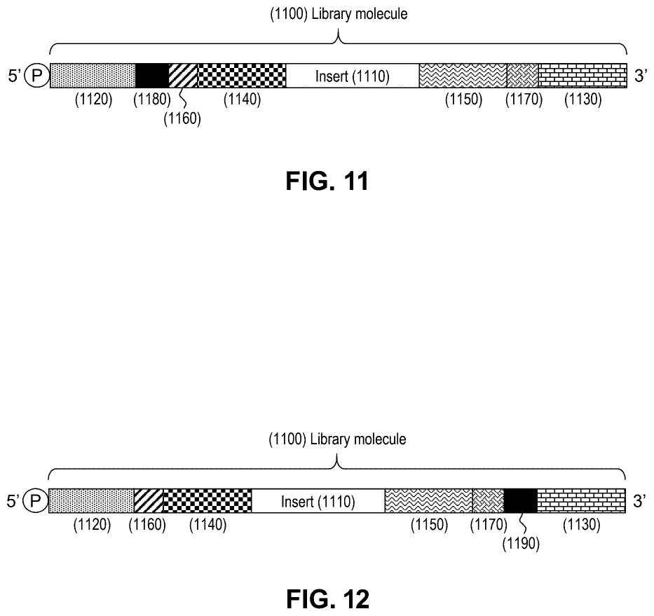

In some embodiments, the immobilized concatemers each comprise tandem repeat units of the sequence-of-interest (e.g., insert region) and any adaptor sequences. For example, the tandem repeat unit comprises: (i) a left universal adaptor sequence having a binding sequence for a first surface primer (1120) (e.g., surface pinning primer), (ii) a left universal adaptor sequence having a binding sequence for a first sequencing primer (1140) (e.g., forward sequencing primer), (iii) a sequence-of-interest (1110), (iv) a right universal adaptor sequence having a binding sequence for a second sequencing primer (1150) (e.g., reverse sequencing primer), (v) a right universal adaptor sequence having a binding sequence for a second surface primer (1130) (e.g., surface capture primer), and (vii) a left sample index sequence (1160) and/or a right sample index sequence (1170). In some embodiments, the tandem repeat unit further comprises a left unique identification sequence (1180) and/or a right unique identification sequence (1190). In some embodiments, the tandem repeat unit further comprises at least one binding sequence for a compaction oligonucleotide. In some embodiments, FIGS. 11 and 12 show linear library molecules or a unit of a concatemer molecule.

### Methods for Conducting In Situ Short Read Sequencing

In the methods described herein, the RNA is not extracted from the cellular sample and sequencing information does not need to be tracked and mapped back to an image of the cellular sample. Rather, RNA is retained inside the cellular sample to permit direct imaging of the spatial location of target RNAs within the cells. Additionally, RNA within the cellular sample is not fragmented and enrichment of target RNA is not necessary. Use of target-specific and/or random-sequence reverse transcription primers enables detection of both poly-A and non-poly-A RNAs in either uni-plex or multi-plex modes.

In some embodiments, the methods comprise repeatedly conducting a short number of sequencing cycles of the same region of the template molecules (e.g., concatemer molecules). By conducting reiterative short sequencing cycles, the RNA content of the cellular sample can be discovered. Compared to long read sequencing workflows, the reiterative short sequencing cycles described herein use a reduced amount of sequencing reagents which reduces cost and saves time. Methods for conducting reiterative short sequencing cycles has many uses including but not limited to detecting specific RNAs of interest, mutant RNA sequences, splice variants, and their abundance levels thereof.

The concatemers carry tandem repeat units of a cDNA-of-interest, the universal sequencing primer binding site, and the target barcode sequence. The concatemers are sequenced inside the cellular sample where a short number of sequencing cycles are conducted for each round and multiple rounds of short read sequencing is conducted. The full length of the target barcode and cDNA region are not sequenced. Instead, at least a portion of the target barcode region is reiteratively sequenced. In some embodiments, it is not necessary to sequence the cDNA region. In some embodiments, the target barcode and a portion of the cDNA region are reiteratively sequenced. It is not necessary to sequence the entire length of the cDNA region. It is not necessary to assemble the sequencing reads or to obtain a full length sequence of the cDNAs-of-interest. The redundant sequencing information obtained from the short sequencing reads obviates the need to sequence the complementary strand of the concatemer. Thus pairwise sequencing is not necessary.

Additionally, a short portion of the cDNA region in the concatemer is re-sequenced at least once (e.g., reiterative sequencing) from the same start position to generate overlapping sequencing reads that can be aligned to a reference sequence. For example, the same portion of the concatemer molecule can be sequenced at least two, three, four, five, or up to 50 times. The start sequencing site can be any location of the concatemer and is dictated by the sequencing primers which are designed to anneal to a selected position within the concatemer. The reiterative short sequencing reads increase the redundancy of sequencing information for individual bases in the cDNA region. Reiteratively sequencing one strand of the concatemer template molecule provides enough base coverage to reveal the presence of target RNAs in the cellular sample so that pairwise sequencing of the complementary strand is not necessary.

A concatemer template molecule includes multiple sequencing primer binding sites along the same concatemer molecule which can be used to generate multiple usable sequencing reads for increased sequencing depth. Together, reiteratively sequencing one strand of the concatemer templates increases sequencing base coverage and sequencing depth compared to sequencing a one-copy template molecule.

The methods described herein can be conducted in uni-plex or multi-plex modes. Two or more different target RNAs can be detected and imaged simultaneously inside a cellular sample using different reverse transcription primers, different target-specific padlock probes, and universal sequencing primers. For example, the presence of a housekeeping RNA and at least one target RNA in a cellular sample can be simultaneously detected and imaged using any of the reiterative short read sequencing methods described herein.

The present disclosure provides methods for detecting in situ at least two different target RNA molecules in a cellular sample comprising step (a): providing a cellular sample harboring a plurality of RNA which comprises at least a first target RNA molecule and a second target RNA molecule. In some embodiments, the cellular sample is fixed and permeabilized. In some embodiments, the cellular sample harbors 2-25 different target RNA molecules, or harbors 25-50 different target RNA molecules, or harbors 50-75 different target RNA molecules, or harbors 75-100 different target RNA molecules. In some embodiments, the cellular sample harbors more than 100 different target RNA molecules, or more than 250 different target RNA molecules, or more than 500 different target molecules, or more than 1000 different target RNA molecules, or more. In some embodiments, the cellular sample harbors more than 10,000 different target RNA molecules. In some embodiments, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, an FFPE cellular sample, or a sectioned FFPE cellular sample. In some embodiments, the cellular sample is deposited onto a solid support. In some embodiments, the cellular sample is deposited onto a solid support which is passivated with a coating that promotes cell adhesion. In some embodiments, the cellular sample is deposited on a support that lacks immobilized capture oligonucleotides. In some embodiments, the cellular sample is cultured before or after depositing the cellular sample onto the solid support. In some embodiments, the cellular sample is cultured prior to conducting step (b) which is described below. In some embodiments, the cellular sample comprises an expanded cellular sample that has been cultured in a simple or complex cell culture media. In some embodiments, the cellular sample is not cultured or expanded prior to conducting step (b).

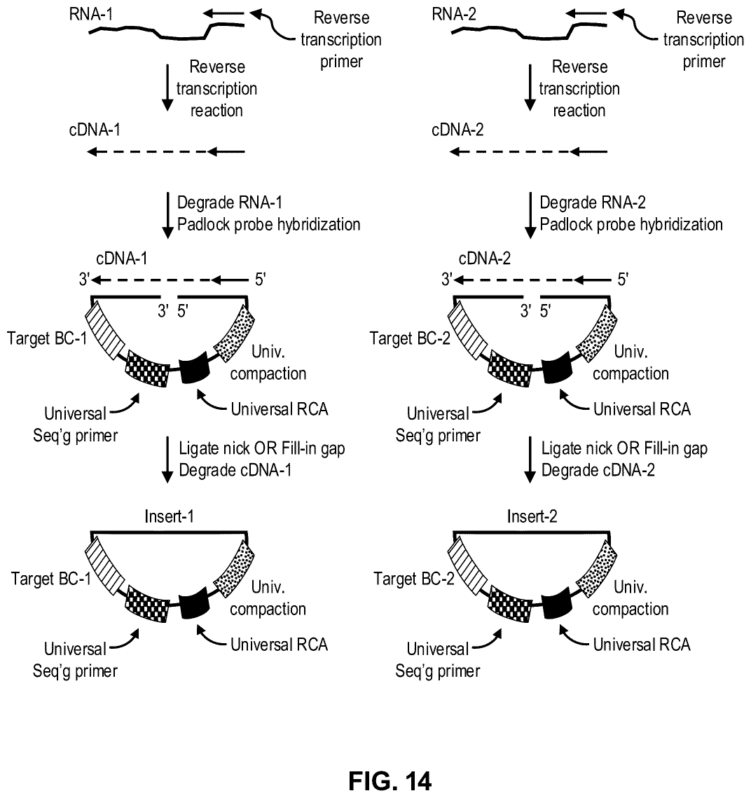

In some embodiments, methods for detecting at least two different target RNA molecules in a cellular sample further comprise step (b): generating inside the cellular sample a plurality of cDNA molecules which include at least a first target cDNA molecule that corresponds to the first target RNA molecule, and the plurality of cDNA molecules includes a second target cDNA molecule that corresponds to the second target RNA molecule. In some embodiments, the method comprises generating at least 2-10,000 different target cDNA molecules that correspond to 2-10,000 different target RNA molecules. In some embodiments, the generating of step (b) comprises contacting the plurality of RNA inside the cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample (e.g., FIG. 14).

In some embodiments, the plurality of reverse transcription primers comprises a first sub-population of target-specific reverse transcription primers that hybridize selectively to the first target RNA, and comprises a second sub-population of target-specific reverse transcription primers that hybridize selectively to the second target RNA. In some embodiments, the first and second sub-population of target-specific reverse transcription primers have the same sequence or different sequences.

In some embodiments, the entire length of the first sub-population of target-specific reverse transcription primers hybridize to a first target RNA molecule. In some embodiments, the first sub-population of target-specific reverse transcription primers comprise tailed primers having a portion that hybridizes to a first target RNA molecule and a portion that does not hybridize to a first target RNA molecule. In some embodiments, the first sub-population of target-specific reverse transcription primers comprise at least a portion having a poly-T sequence. In some embodiments, the first sub-population of target-specific reverse transcription primers comprise at least a portion having a random sequence and/or at least a portion having a target-specific sequence.

In some embodiments, the entire length of the second sub-population of target-specific reverse transcription primers hybridize to a second target RNA molecule. In some embodiments, the second sub-population of target-specific reverse transcription primers comprise tailed primers having a portion that hybridizes to a second target RNA molecule and a portion that does not hybridize to a second target RNA molecule. In some embodiments, the second sub-population of target-specific reverse transcription primers comprise at least a portion having a poly-T sequence. In some embodiments, the second sub-population of target-specific reverse transcription primers comprise at least a portion having a random sequence and/or at least a portion having a target-specific sequence.

In some embodiments, a target RNA molecule that is hybridized to a cDNA molecule can be subjected to enzymatic degradation using a ribonuclease under a condition suitable for degrading RNA in an RNA/DNA duplex. In some embodiments, a target RNA molecule that is hybridized to a cDNA molecule is not subjected to enzymatic degradation.

In some embodiments, methods for detecting at least two different target RNA molecules in a cellular sample further comprise step (c): contacting the plurality of cDNA molecules in the cellular sample with a plurality of target-specific padlock probes which includes at least a first plurality of target-specific padlock probes and a second plurality of target-specific padlock probes. In some embodiments, the method comprises contacting the plurality of cDNA molecule in the cellular sample with at least 2-10,000 different target-specific padlock probes.

In an alternative embodiment, cDNA is not generated from RNA inside the cellular sample. In some embodiments, methods for detecting at least two different target RNA molecules in a cellular sample further comprise contacting RNA inside the cell with a plurality of target-specific padlock probes and generating circularized padlock probes. In some embodiments, methods for detecting at least two different target RNA molecules in a cellular sample further comprise step (c): contacting the plurality of RNA molecules in the cellular sample with a plurality of target-specific padlock probes which includes at least a first plurality of target-specific padlock probes and a second plurality of target-specific padlock probes. In some embodiments, the method comprises contacting the plurality of cDNA molecule in the cellular sample with at least 2-10,000 different target-specific padlock probes. In some embodiments, a target RNA molecule can be subjected to enzymatic degradation using a ribonuclease. In some embodiments, a target RNA molecule is not subjected to enzymatic degradation.

In some embodiments, individual padlock probes in the plurality of first target-specific padlock probes comprise first and second terminal regions (e.g., first and second padlock binding arms), wherein the first terminal region selectively hybridizes to a first region of the first target cDNA molecule (or the first target RNA molecule), and the second terminal region selectively hybridizes to a second region of the first target cDNA molecule (or the first target RNA molecule). In some embodiments, the contacting of step (c) comprises: hybridizing the first and second terminal regions of the first target-specific padlock probes to proximal positions on the first target cDNA molecule (or the first target RNA molecule) to form a circularized first target-specific padlock probe having a nick or gap between the hybridized first and second terminal regions (e.g., FIG. 14, left). In some embodiments, the first target-specific padlock probe comprises a first target barcode sequence (target BC-1) that corresponds to and uniquely identifies the first target cDNA sequence (or the first target RNA sequence). In some embodiments, the first target-specific padlock probe comprises a first target barcode sequence that is located adjacent to one of the regions of the first target-specific padlock probe that selectively hybridizes to the first target cDNA molecule (or the first target RNA sequence). In some embodiments, the first target-specific padlock probe comprises at least one universal adaptor sequence, such as for example a universal sequencing primer binding site (or a complementary sequence thereof). In some embodiments, the first target-specific padlock probe comprises a universal primer binding site for a rolling circle amplification primer (or a complementary sequence thereof). In some embodiments, the first target-specific padlock probe comprises a universal compaction oligonucleotide binding site (or a complementary sequence thereof).

In some embodiments, individual padlock probes in the plurality of second target-specific padlock probes comprise first and second terminal regions (e.g., first and second padlock binding arms), wherein the first terminal region selectively hybridizes to a first region of the second target cDNA molecule (or the second target RNA molecule), and the second terminal region selectively hybridizes to a second region of the second target cDNA molecule (or the second target RNA molecule). In some embodiments, the contacting of step (c) comprises: hybridizing the first and second terminal regions of the second target-specific padlock probes to proximal positions on the second target cDNA molecule (or the second target RNA molecule) to form a circularized second target-specific padlock probe having a nick or gap between the hybridized first and second terminal regions (e.g., FIG. 14, right). In some embodiments, the second target-specific padlock probe comprises a second target barcode sequence (target BC-2) that corresponds to and uniquely identifies the second target cDNA sequence (or the second target RNA sequence). In some embodiments, the second target-specific padlock probe comprises a second target barcode sequence that is located adjacent to one of the regions of the second target-specific padlock probe that selectively hybridizes to the second target cDNA molecule (or the second target RNA sequence). In some embodiments, the second target-specific padlock probe comprises at least one universal adaptor sequence, such as for example a universal sequencing primer binding site (or a complementary sequence thereof). In some embodiments, the second target-specific padlock probe comprises a universal primer binding site for a rolling circle amplification primer (or a complementary sequence thereof). In some embodiments, the second target-specific padlock probe comprises a universal compaction oligonucleotide binding site (or a complementary sequence thereof).

In some embodiments, the first target barcode sequence (target BC-1) and the second target barcode sequence (target BC-2) have different sequences and can be used to conduct multiplex RNA detection and sequencing. In some embodiments, the first target barcode sequence (target BC-1) and the second target barcode sequence (target BC-2) have the same sequence and can be used to conduct uni-plex RNA detection and sequencing.

In some embodiments, the first and second target-specific padlock probes comprise a universal sequencing primer binding site and a target barcode sequence that are adjacent to each other so that the target barcode region of the concatemer is sequenced first. The target barcode sequence can be any length, for example 3-15 bases, or 15-25 bases, or 25-40 bases, or longer.

In some embodiments, methods for detecting at least two different target RNA molecules in a cellular sample further comprising step (d): closing the nick or gap in the at least first and second circularized target-specific padlock probes by conducting an enzymatic reaction, thereby generating at least a first covalently closed circular padlock probe and a second covalently closed circular padlock probe inside the cellular sample. In some embodiments, the closing the nick in the first and second circularized padlock probes comprises conducting an enzymatic ligation reaction. In some embodiments, closing the gap in the first and second circularized padlock probes comprises conducting a polymerase-catalyzed fill-in reaction using the first or second target cDNA molecule (or the first or second RNA molecule) as a template, and conducting an enzymatic ligation reaction. In some embodiments, the method comprises closing the nick or gap in at least 2-10,000 circularized target-specific padlock probes by conducting one or more enzymatic reactions, thereby generating at least 2-10,000 covalently closed circular padlock probes inside the cellular sample.

In some embodiments, methods for detecting at least two different target RNA molecules in a cellular sample further comprising step (e): conducting a rolling circle amplification reaction inside the cellular sample using the first and second covalently closed circular padlock probes as template molecules, thereby generating a plurality of concatemer molecules including at least a first concatemer molecule that corresponds to a first target RNA molecule, and the plurality of concatemer molecules includes at least a second concatemer molecule that corresponds to a second target RNA molecule. In some embodiments, the first concatemer molecule comprises tandem repeat units, wherein a unit comprises a sequence that corresponds to the first target cDNA (or the first target RNA), the first target barcode sequence, and the universal sequencing primer binding site (or a complementary sequence thereof). In some embodiments, the second concatemer molecule comprises tandem repeat units, wherein a unit comprises a sequence that corresponds to the second target cDNA (or the second target RNA), the second target barcode sequence, and the universal sequencing primer binding site (or a complementary sequence thereof).

In some embodiments, the rolling circle amplification reaction of step (e) comprises contacting the covalently closed circularized padlock probes with an amplification primer (e.g., a universal rolling circle amplification primer), a strand-displacing DNA polymerase, and a plurality of nucleotides, under a condition suitable for hybridizing individual amplification primers to a covalently closed padlock probe, and under a condition suitable for conducting primer extension using the covalently closed padlock probe as a template molecule to generate a nucleic acid concatemer. In some embodiments, the method comprises conducting a rolling circle amplification reaction inside the cellular sample using the at least 2-10,000 covalently closed circular padlock probes as template molecules, thereby generating at least 2-10,000 concatemer molecules that correspond to at least 2-10,000 target RNA molecules. In some embodiments, the plurality of concatemers that are generated inside the cellular sample collapse into a DNA nanoball having a shape and size that is more compact compared to a non-collapsed concatemer.

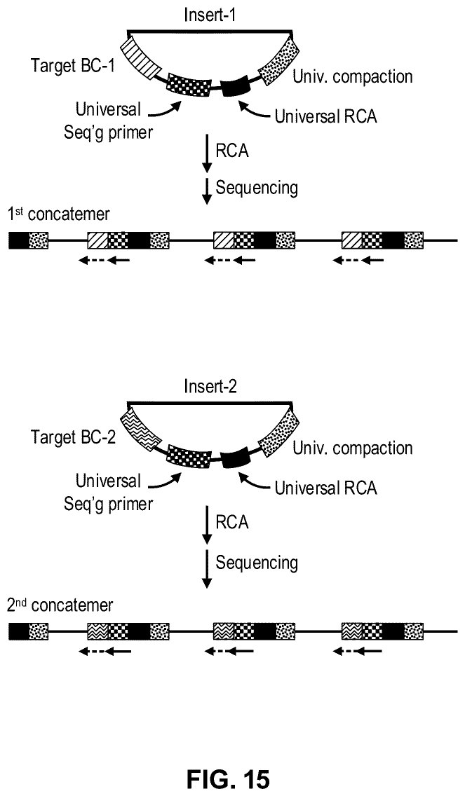

In some embodiments, methods for detecting at least two different target RNA molecules in a cellular sample further comprising step (f): sequencing the plurality of concatemer molecules inside the cellular sample, which comprises sequencing the first concatemer molecule by conducting 2-1000 sequencing cycles to generate a plurality of first sequencing read products, and sequencing the second concatemer molecule by conducting 2-1000 sequencing cycles to generate a plurality of second sequencing read products (FIG. 15). In some embodiments, the sequencing of step (f) comprises sequencing no more than 2-30 bases of the first concatemer molecules to generate a plurality of first sequencing read products, and which comprises sequencing no more than 2-30 bases of the second concatemer molecules to generate a plurality of second sequencing read products. In some embodiments, the method comprises sequencing the at least 2-10,000 concatemer molecules inside the cellular sample, which comprises conducting 2-1000 sequencing cycles on the 2-10,000 concatemer molecules to generate a plurality of sequencing read products.

In some embodiments, only the first target barcode region of the first concatemer molecules are sequenced (e.g., FIG. 15, top). In some embodiments, at least a portion or the full length of the first target barcode of the first concatemer molecules are sequenced (e.g., FIG. 15, top). In some embodiments, the first target barcode is sequenced and a portion of the first cDNA region (or the first RNA region) of the first concatemer molecules are sequenced. In some embodiments, at least a portion of the first cDNA region (or the first RNA region) of the first concatemer molecules are sequenced.

In some embodiments, only the second target barcode region of the second concatemer molecules are sequenced (e.g., FIG. 15, bottom). In some embodiments, at least a portion or the full length of the second target barcode of the second concatemer molecules are sequenced (e.g., FIG. 15, bottom). In some embodiments, the second target barcode is sequenced and a portion of the second cDNA region (or the second RNA region) of the second concatemer molecules are sequenced. In some embodiments, at least a portion of the second cDNA region (or the second RNA region) of the second concatemer molecules are sequenced.

In some embodiments, the sequencing of step (f) comprises contacting the plurality of concatemer molecules inside the cellular sample with (i) a plurality of universal sequencing primers, (ii) a plurality of sequencing polymerases, and (iii) a plurality of nucleotide reagents, under a condition suitable for hybridizing the plurality of universal sequencing primers to their respective universal sequencing primer binding sites on the concatemers. In some embodiments, the sequencing of step (f) further comprises conducting 2-1000 sequencing cycles to generate at least a first plurality of sequencing read products by sequencing at least the first target barcode region (Target BC-1), and optionally conducting 2-1000 sequencing cycles to generate at least a second plurality of sequencing read products by sequencing at least the second target barcode region (Target BC-2). In some embodiments, the nucleotide reagents comprise multivalent molecules, nucleotides and/or nucleotide analogs.

In some embodiments, the sequencing of step (f) comprises sequencing at least a portion of the first and second nucleic acid concatemers using an optical imaging system comprising a field-of-view (FOV) greater than 1.0 mm².

In some embodiments, in the sequencing of step (f), the plurality of first and second sequencing read products are detectable by imaging, and wherein the sequencing comprises decoding the plurality of first and second sequencing read products from the images obtained during the no more than 2-1000 sequencing cycles.

In some embodiments, in the sequencing of step (f), the plurality of the first and second sequencing read products are detectable by imaging, and wherein the sequencing comprises simultaneously imaging the plurality of first and second detectable sequencing read products in the cellular sample (co-localization of the first and second sequencing read products).

In some embodiments, methods for detecting at least two different target RNA molecules in a cellular sample further comprising step (g): removing the plurality of first sequencing read products from the first concatemer molecules and retaining the first concatemer molecules in the cellular sample, and removing the plurality of second sequencing read products from the second concatemer molecules and retaining the second concatemer molecules in the cellular sample.

In some embodiments, methods for detecting at least two different target RNA molecules in a cellular sample further comprising step (h): reiteratively sequencing the plurality of concatemers by repeating steps (f) and (g) at least once, wherein the sequences of the plurality of first sequencing read products confirms the presence of the first target RNA molecules in the cellular sample, and wherein the sequences of the plurality of second sequencing read products confirms the presence of the second target RNA molecules in the cellular sample.

In some embodiments, reiteratively sequencing at least one region of the concatemer comprises repeating steps (f)-(g) at least 2 times, at least 3 times, at least 4 times, at least 5 times, at least 6 times, at least 7 times, at least 8 times, at least 9 times, or at least 10 times.

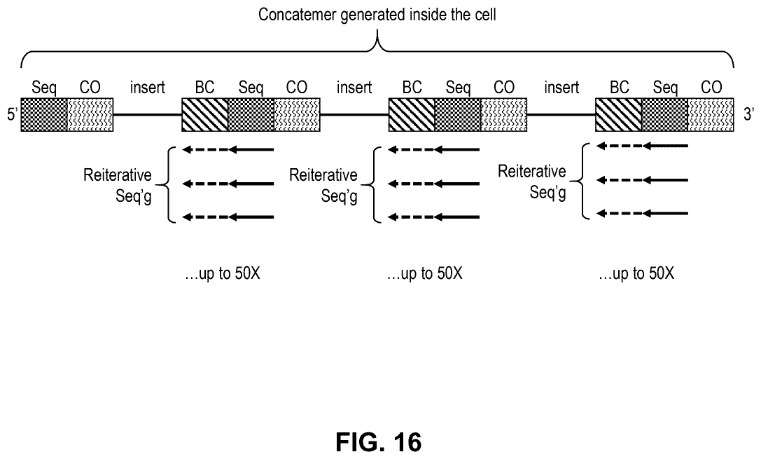

In some embodiments, reiteratively sequencing at least one region of the concatemer comprises repeating steps (f)-(g) up to 10 times, up to 20 times, up to 30 time, up to 40 times, or up to 50 times. An example of reiterative sequence is shown in a schematic in FIGS. 16-19.

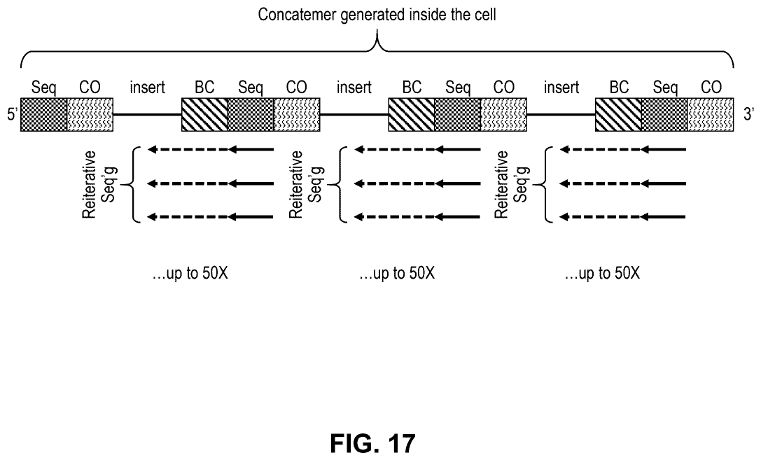

In some embodiments, e.g., as shown in FIG. 17, universal sequencing primers (solid arrows) hybridize to the universal sequencing primer binding sites and up to 1000 sequencing cycles are conducted to generate a plurality of first sequencing read products (dashed arrows), where the first sequencing read products include only the target barcode sequence. The plurality of first sequencing read products are removed from the concatemer, and the sequencing is repeated where up to 1000 sequencing cycles are conducted to generate another plurality of first sequencing read products (dashed arrows), where the first sequencing read products include only the target barcode sequence. The plurality of first sequencing read products are removed from the concatemer, and the sequencing is once again repeated where up to 1000 sequencing cycles are conducted to generate another plurality of first sequencing read products (dashed arrows), where the first sequencing read products include only the target barcode sequence. In some embodiments, the reiterative sequencing can be conducted up to 50 times. The sequences of all of the first sequencing read products can be determined and aligned with a first reference sequence (e.g., reference barcode sequence) to confirm the presence of the first target RNA molecules inside the cellular sample.

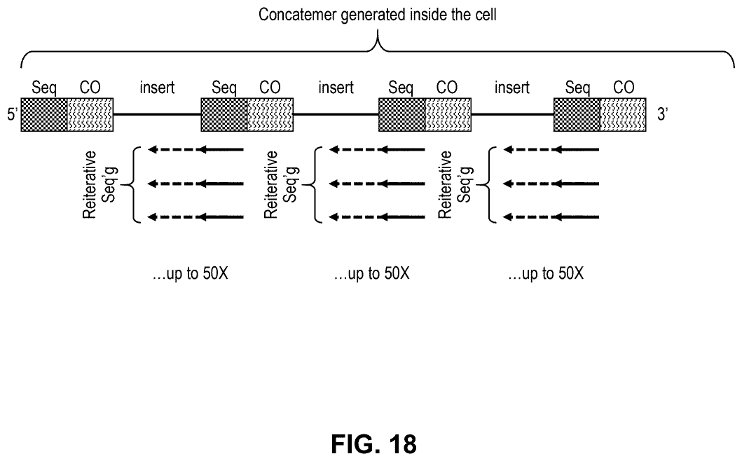

In some embodiments, e.g., in FIG. 18, universal sequencing primers (solid arrows) hybridize to the universal sequencing primer binding sites and up to 1000 sequencing cycles are conducted to generate a plurality of first sequencing read products (dashed arrows), where the first sequencing read products include a portion of the insert sequence. The plurality of first sequencing read products are removed from the concatemer, and the sequencing is repeated where up to 1000 sequencing cycles are conducted to generate another plurality of first sequencing read products (dashed arrows), where the first sequencing read products include a portion of the insert sequence. The plurality of first sequencing read products are removed from the concatemer, and the sequencing is once again repeated where up to 1000 sequencing cycles are conducted to generate another plurality of first sequencing read products (dashed arrows), where the first sequencing read products include a portion of the insert sequence. In some embodiments, the reiterative sequencing can be conducted up to 50 times. The sequences of all of the first sequencing read products can be determined and aligned with a first reference sequence (e.g., the insert sequence that corresponds to the target RNA) to confirm the presence of the first target RNA molecules inside the cellular sample.

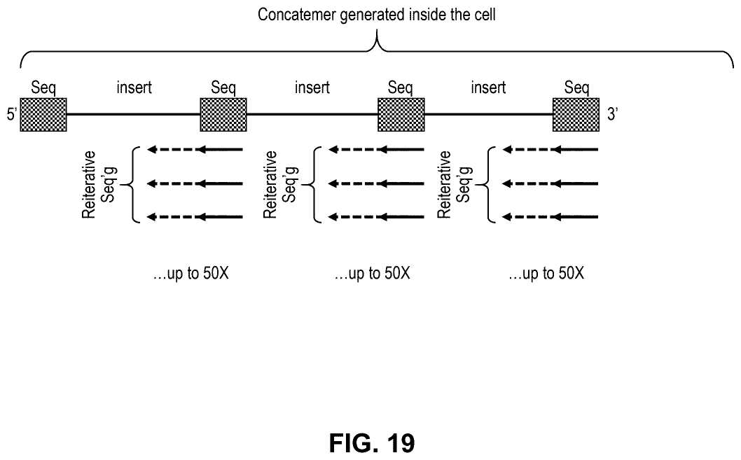

In some embodiments, e.g., FIG. 19, universal sequencing primers (solid arrows) hybridize to the universal sequencing primer binding sites and up to 1000 sequencing cycles are conducted to generate a plurality of first sequencing read products (dashed arrows), where the first sequencing read products include a portion of the insert sequence. The plurality of first sequencing read products are removed from the concatemer, and the sequencing is repeated where up to 1000 sequencing cycles are conducted to generate another plurality of first sequencing read products (dashed arrows), where the first sequencing read products include a portion of the insert sequence. The plurality of first sequencing read products are removed from the concatemer, and the sequencing is once again repeated where up to 1000 sequencing cycles are conducted to generate another plurality of first sequencing read products (dashed arrows), where the first sequencing read products include a portion of the insert sequence. In some embodiments, the reiterative sequencing can be conducted up to 50 times. The sequences of all of the first sequencing read products can be determined and aligned with a first reference sequence (e.g., the insert sequence that corresponds to the target RNA) to confirm the presence of the first target RNA molecules inside the cellular sample.

In some embodiments, at least one concatemer is sequenced by conducting step (f) once (non-reiterative sequencing). In some embodiments, at least one concatemer is sequenced by conducting steps (f)-(g) once. In some embodiments, at least one concatemer is reiteratively sequenced by conducting steps (f)-(g) at least twice.

In some embodiments, the plurality of universal sequencing primers can be hybridized to concatemer template molecules with a hybridization reagent comprising an SSC buffer (e.g., 2× saline-sodium citrate) buffer with formamide (e.g., 10-20% formamide). The hybridization conditions comprise a temperature of about 20-30° C., for about 10-60 minutes.

In some embodiments, the plurality of sequencing read products can be removed from the concatemers and the plurality of concatemers can be retained inside the cellular sample using a de-hybridization reagent comprising an SSC buffer (e.g., saline-sodium citrate) buffer, with or without formamide, at a temperature that promotes nucleic acid denaturation such as for example 30-90° C.

In some embodiments, the plurality of nucleotide reagents of step (f) comprise a plurality of nucleotides that are detectably labeled or non-labeled. In some embodiments, individual nucleotides are linked to a detectable reporter moiety. In some embodiments, the detectable reporter moiety comprises a fluorophore. In some embodiments, the plurality of detectably labeled nucleotide analogs comprise a plurality of chain terminating nucleotides, where the chain terminating moiety is linked to 3′ nucleotide sugar position to form a 3′ blocked nucleotide analog. In some embodiments, the chain terminating moiety can be removed to convert 3′ blocked nucleotide analog to an extendible nucleotide having a 3′ OH group on the sugar. In some embodiments, the labeled nucleotide analogs are linked to a different fluorophore that corresponds to the nucleo-bases adenine, cytosine, guanine, thymine or uracil, where the different fluorophores emit a fluorescent signal during the sequencing of step (f). In some embodiments, a sequencing cycle comprises (1) contacting the concatemer/sequencing primer duplex with a sequencing polymerase and a detectably labeled chain terminating nucleotide under a condition suitable for polymerase-catalyzed incorporation of the detectably labeled chain terminating nucleotide into the terminal end of the sequencing primer, (2) detecting and imaging the fluorescent signal and color emitted by the incorporated chain terminating nucleotide, and (3) removing the chain terminating moiety (e.g., unblocking) and the fluorophore from the incorporated nucleotide and retaining the concatemer/sequencing primer duplex. In some embodiments, no more than 2-1000 sequencing cycles are conducted on the plurality of concatemers inside the cellular sample to generate a plurality of sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the second target RNA molecules inside the cellular sample.

In some embodiments, the sequences of the first and second sequencing read products can be aligned after each round of generating the first and second sequencing read products which are no more than 30 to 1000 bases in length, or after generating a set of reiterative sequencing read products wherein the first and second sequencing read products which are no more than 30 to 1000 bases in length. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules. In some embodiments, when the sequencing is conducted using different fluorescently-labeled nucleotide reagents that correspond to different nucleo-bases (e.g., A, G, C, T/U), then the images can have different color fluorescent spots co-located in the same cellular sample at different sequencing cycles.

In some embodiments, out-of-sync phasing and/or pre-phasing events can occur during synchronized sequencing reactions on clonally amplified template amplicons, where the sequencing reactions comprise polymerase-catalyzed sequencing reactions employing detectably labeled chain terminator nucleotides. In some embodiments, a sequencing reaction on one template molecule in the clonally-amplified template molecules moves ahead (e.g., pre-phasing) or fall behind (e.g., phasing) of the sequencing of the other template molecules within the clonally-amplified template molecules. During sequencing, a fluorescent signal is typically detected which corresponds to incorporation of a labeled chain terminator nucleotide. Thus, phasing and pre-phasing events can be detected and monitored using incorporation of a labeled chain terminator nucleotide.

In some embodiments, the plurality of nucleotide reagents of step (f) comprise a plurality of multivalent molecules each comprising a core attached to a plurality of nucleotide-arms, wherein the nucleotide-arms are attached to a nucleotide unit. In some embodiments, individual multivalent molecules are labeled with a detectably reporter moiety. In some embodiments, the detectable reporter moiety comprises a fluorophore. In some embodiments, the core of the multivalent molecule is labeled with a fluorophore, and wherein the fluorophore which is attached to a given core of the multivalent molecule corresponds to the nucleotide base (e.g., adenine, guanine, cytosine, thymine or uracil) of the nucleotide arm. In some embodiments, at least one of the nucleotide arms of the multivalent molecule comprises a linker and/or nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments, a sequencing cycle comprises (1) contacting the concatemer/sequencing primer duplex with a first sequencing polymerase to form a complexed polymerase, (2) contacting the complexed polymerase with a detectably labeled multivalent molecule under a condition suitable for binding a complementary nucleotide unit of the multivalent molecule to the complexed polymerase thereby forming a multivalent-binding complex, and the condition is suitable for inhibiting incorporation of the complementary nucleotide unit into the terminal end of the sequencing primer, (3) detecting and imaging the fluorescent signal and color emitted by the bound detectably labeled multivalent molecule, (4) removing the first sequencing polymerase and the bound detectably labeled multivalent molecule, and retaining the concatemer/sequencing primer duplex, (5) contacting the retained concatemer/sequencing primer duplex with a second sequencing polymerase and a non-labeled chain terminating nucleotide under a condition suitable for polymerase-catalyzed incorporation of the non-labeled chain terminating nucleotide into the terminal end of the sequencing primer, and (6) removing the chain terminating moiety (e.g., unblocking) and retaining the concatemer/sequencing primer duplex. In some embodiments, no more than 2-1000 sequencing cycles are conducted on the plurality of concatemers inside the cellular sample to generate a plurality of sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the second target RNA molecules inside the cellular sample. In some embodiments, the sequences of the first and second sequencing read products can be aligned after each round of generating the first and second sequencing read products which are no more than 30 to 1000 bases in length, or after generating a set of reiterative sequencing read products wherein the first and second sequencing read products which are no more than 150 or no more than 1000 bases in length. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules. In some embodiments, individual cycle times can be achieved in less than 30 minutes. In some embodiments, the field of view (FOV) can exceed 1 mm² and the cycle time for scanning large area (>10 mm²) can be less than 5 minutes.

In some embodiments, when sequencing with detectably labeled multivalent molecules, step (2) in which multivalent-binding complexes are formed and step (3) in which the bound detectably labeled multivalent molecules are imaged and detected, the conditions are gentle compared to sequencing workflows that employ detectable labeled chain terminating nucleotides. For example, steps (2) and (3) can be conducted at a gentle temperature of about 35-45° C., or about 39-42° C. Steps (2) and (3) can be conducted at a gentle temperature which can help retain the compact size and shape of a DNA nanoball during multiple sequencing cycles (e.g., up to 30 cycles) which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

In some embodiments, out-of-sync phasing and/or pre-phasing events can occur during synchronized polymerase-catalyzed sequencing reactions employing detectably labeled multivalent molecules. During sequencing, a fluorescent signal can be detected which corresponds to binding of complementary nucleotide unit of a multivalent molecule to the complexed polymerase thereby forming a multivalent-binding complex. Thus, phasing and pre-phasing events can be detected and monitored using binding of labeled multivalent molecules. In some embodiments, when conducting up to 30 or 150 sequencing cycles with detectably labeled multivalent molecules, the phasing and/or pre-phasing rate can be less than about 5%, or less than about 1%, or less than about 0.01%, or less than about 0.001%. By contrast, the phasing and/or pre-phasing rates for conducting up to 30 sequencing cycles using labeled chain terminator nucleotides can be about 5%.

### Methods for Conducting In Situ RNA Batch Sequencing

The present disclosure provides methods for conducting in situ multiplex and multi-omics detection and identification using coded padlocks probes. The padlock probes are designed to selectively detect target RNA.

The RNA-specific padlock probes selectively hybridize to cDNA that corresponds to target RNA. The RNA-specific probes carry barcodes that uniquely identify the cDNA. In some embodiments, the RNA-specific padlock probes also carry batch-specific sequencing primer binding sites.

Both types of padlock probes are used to generate concatemers which having multiple copies of batch-specific sequencing binding sites and barcodes. The concatemers can collapse into DNA nanoballs having compact shape and size that produce increased signal intensity and color differentiation during sequencing.

For in situ sequencing, the limit of optical resolution impedes the ability to perform highly multiplex sequencing. The batch-specific sequencing primer binding sites on the padlock probes enables sequencing a desired subset (e.g., a batch) of the concatemers using selected batch-specific sequencing primers to reduce over-crowding signals and images. The use of batch-specific sequencing primers produces optical images that are intense and resolvable. By conducting multiple rounds of sequencing on the same cellular sample using different batch-specific sequencing primers enables multiplex sequencing to reveal numerous target RNAs.

The batch-specific sequencing methods described herein have many uses. For example, the number of spots that are imaged and associated with sequencing can be counted. The counted spots can be used as a measure of RNA levels in a cellular sample.

The present disclosure provides methods for detecting in situ at least two different target RNA molecules, comprising step (a): providing a cellular sample deposited on a solid support, wherein the cellular sample harbors (i) a first plurality of DNA amplicons (e.g., first concatemers) that correspond to a first target cDNA or RNA molecule, and (ii) a second plurality of DNA amplicons (e.g., second concatemers) that correspond to a second target cDNA or RNA molecule.

In some embodiments, the method further comprises step (b): sequencing the first plurality of DNA amplicons inside the cellular sample under a condition that inhibits sequencing the second plurality of DNA amplicons, wherein sequencing the first plurality of DNA amplicons inside the cellular sample comprises generating a plurality of first sequencing read products, wherein the sequences of the first sequencing read products are aligned with a first target reference sequence to confirm the presence of the first target RNA in the cellular sample. In some embodiments, the first amplicons can be reiteratively sequenced by conducting 2-1000 sequencing cycles, or can be reiteratively sequenced by conducting 1-250 sequencing cycles.

In some embodiments, the method further comprises step (c): sequencing the second plurality of DNA amplicons inside the cellular sample under a condition that inhibits sequencing the first plurality of DNA amplicons, wherein sequencing the second plurality of DNA amplicons inside the cellular sample comprises generating a plurality of second sequencing read products, wherein the sequences of the second sequencing read products are aligned with a second target reference sequence to confirm the presence of the second target RNA in the cellular sample. In some embodiments, the second amplicons can be reiteratively sequenced by conducting 2-1000 sequencing cycles, or can be reiteratively sequenced by conducting 1-250 sequencing cycles.

The present disclosure provides methods for detecting in situ at least two different target RNA molecules, comprising step (a): providing a cellular sample deposited on a solid support, wherein the cellular sample harbors a first plurality of target RNA and a second plurality of target RNA. In some embodiments, the first plurality of target RNA encode a first polypeptide. In some embodiments, the second plurality of target RNA encode a second polypeptide. In some embodiments, the cellular sample is fixed and permeabilized.

In some embodiments, the cellular sample harbors 2-25 different target RNA molecules, or harbors 25-50 different target RNA molecules, or harbors 50-75 different target RNA molecules, or harbors 75-100 different target RNA molecules. In some embodiments, the cellular sample harbors more than 100 different target RNA molecules, or more than 250 different target RNA molecules, or more than 500 different target molecules, or more than 1000 different target RNA molecules, or more. In some embodiments, the cellular sample harbors more than 10,000 different target RNA molecules. In some embodiments, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, or an FFPE cellular sample. In some embodiments, the cellular sample is deposited onto a solid support. In some embodiments, the cellular sample is deposited onto a solid support which is passivated with a coating that promotes cell adhesion. In some embodiments, the cellular sample is deposited on a support that lacks immobilized capture oligonucleotides. In some embodiments, the cellular sample is cultured prior to conducting step (b) which is described below.

In some embodiments, the cellular sample harbors 2-25 different target polypeptide molecules, or harbors 25-50 different target polypeptide molecules, or harbors 50-75 different target polypeptide molecules, or harbors 75-100 different target polypeptide molecules. In some embodiments, the cellular sample harbors more than 100 different target polypeptide molecules, or more than 250 different target polypeptide molecules, or more than 500 different target molecules, or more than 1000 different target polypeptide molecules, or more. In some embodiments, the cellular sample harbors more than 10,000 different target polypeptide molecules. The target polypeptide molecules are encoded by the target RNA molecules.

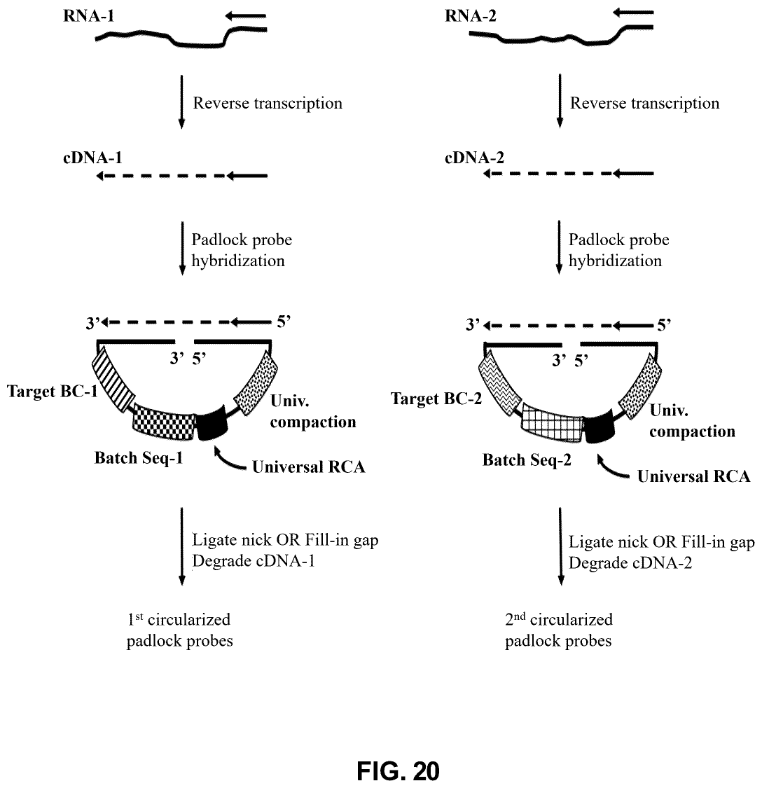

In some embodiments, the methods comprise step (b): generating inside the cellular sample a plurality of cDNA by (i) generating at least a first plurality of target cDNA from the first plurality of target RNA, and (ii) generating at least a second plurality of target cDNA from the second plurality of target RNA (e.g., FIG. 20). In some embodiments, the first target cDNAs correspond to the first target RNA molecules. In some embodiments, the second target cDNAs correspond to the second target RNA molecules. In some embodiments, the method comprises generating at least 2-10,000 different target cDNA molecules that correspond to 2-10,000 different target RNA molecules. In some embodiments, the generating of step (b) comprises contacting the plurality of RNA inside the cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample. In some embodiments, the plurality of reverse transcription primers comprises a first sub-population of target-specific reverse transcription primers that hybridize selectively to the first target RNA, and/or comprises a second sub-population of target-specific reverse transcription primers that hybridize selectively to the second target RNA. In some embodiments, the plurality of reverse transcription primers comprises a first sub-population of random-sequence reverse transcription primers that hybridize to the first target RNA, and/or comprises a second sub-population of random-sequence reverse transcription primers that hybridize to the second target RNA.

In some embodiments, the methods comprise step (c): generating inside the cellular sample a plurality of DNA concatemers which correspond to the first and second plurality of target RNA molecules, comprising: (1) generating a first plurality of covalently closed circular padlock probes by contacting the first plurality of target cDNA with a first plurality of padlock probes, wherein the contacting is conducted under a condition suitable for hybridizing the first and second binding arms of the first padlock probes to proximal positions on their respective first target cDNA molecules to form a first plurality of circularized padlock probes each having a nick or gap between the hybridized first and second binding arms, wherein the first padlock probes include a (i) a first target barcode sequence (target BC-1) that uniquely identifies the first target RNA or cDNA, (ii) a first batch-specific sequencing primer binding site (Batch Seq-1) (or a complementary sequence thereof), and (iii) a universal binding site for an amplification primer (universal RCA) (or a complementary sequence thereof) (e.g., FIG. 20, left side); (2) enzymatically closing the nick or gap in the first plurality of covalently closed circular padlock probes to form a first plurality of covalently closed padlock probes; and (3) conducting rolling circle amplification inside the cellular sample using the first covalently closed circular padlock probes as template molecules, thereby generating a first plurality of concatemer molecules that correspond to the first plurality of target RNA or cDNA molecules. In some embodiments, the rolling circle amplification reaction can be conducted in the presence or absence of a plurality of compaction oligonucleotides. In some embodiments, the method comprises contacting the plurality of cDNA molecule in the cellular sample with at least 2-10,000 different target-specific padlock probes. In some embodiments, the first padlock probe further comprises a universal compaction oligonucleotide binding site (or a complementary sequence thereof). In some embodiments, the closing the nick in the first circularized padlock probes comprises conducting an enzymatic ligation reaction. In some embodiments, closing the gap in the first circularized padlock probes comprises conducting a polymerase-catalyzed fill-in reaction using the first target cDNA molecule as a template, and conducting an enzymatic ligation reaction. In some embodiments, the method comprises closing the nick or gap in at least 2-10,000 circularized target-specific padlock probes by conducting an enzymatic reaction, thereby generating at least 2-10,000 covalently closed circular padlock probes inside the cellular sample. In some embodiments, each concatemer molecule in the first plurality comprises tandem repeat units, wherein a unit comprises the sequence of the first target cDNA and (i) the first target barcode sequence (target BC-1) that uniquely identifies the first target RNA, (ii) the first batch-specific sequencing primer binding site (Batch Seq-1) (or a complementary sequence thereof), and (iii) the universal binding site for an amplification primer (universal RCA) (or a complementary sequence thereof). In some embodiments, the unit further comprises the universal compaction oligonucleotide binding site (or a complementary sequence thereof).

In some embodiments, step (c) further comprises: generating inside the cellular sample a plurality of DNA concatemers which correspond to the second plurality of target RNA molecules, comprising: (1) generating a second plurality of covalently closed circular padlock probes by contacting the second plurality of target cDNA with a second plurality of padlock probes, wherein the contacting is conducted under a condition suitable for hybridizing the first and second binding arms of the second padlock probes to proximal positions on their respective second target cDNA molecules to form a second plurality of circularized padlock probes each having a nick or gap between the hybridized first and second binding arms, wherein the second padlock probes include a (i) a second barcode sequence (target BC-2) that uniquely identifies the second target cDNA or RNA, (ii) a second batch-specific sequencing primer binding site (Batch Seq-2) (or a complementary sequence thereof) wherein the sequence of the second batch-specific sequencing primer binding site differs from the sequence of the first batch-specific sequencing primer binding site, and (iii) the universal binding site for an amplification primer (universal RCA) (or a complementary sequence thereof) (e.g., FIG. 20, right side); (2) enzymatically closing the nick or gap in the second plurality of covalently closed circular padlock probes to form a second plurality of covalently closed padlock probes; and (3) conducting rolling circle amplification inside the cellular sample using the second covalently closed circular padlock probes as template molecules, thereby generating a second plurality of concatemer molecules that correspond to the second plurality of target RNA molecules. In some embodiments, the rolling circle amplification reaction can be conducted in the presence or absence of a plurality of compaction oligonucleotides. In some embodiments, the method comprises contacting the plurality of cDNA molecule in the cellular sample with at least 2-10,000 different target-specific padlock probes. In some embodiments, the second padlock probe further comprises a universal compaction oligonucleotide binding site (or a complementary sequence thereof). In some embodiments, the closing the nick in the second circularized padlock probes comprises conducting an enzymatic ligation reaction. In some embodiments, closing the gap in the second circularized padlock probes comprises conducting a polymerase-catalyzed fill-in reaction using the second target cDNA molecule as a template, and conducting an enzymatic ligation reaction. In some embodiments, the method comprises closing the nick or gap in at least 2-10,000 circularized target-specific padlock probes by conducting an enzymatic reaction, thereby generating at least 2-10,000 covalently closed circular padlock probes inside the cellular sample. In some embodiments, each concatemer molecule in the second plurality comprises tandem repeat units, wherein a unit comprises the sequence of the second target cDNA and (i) the second target barcode sequence (target BC-2) that uniquely identifies the second target cDNA or RNA, (ii) the second batch-specific sequencing primer binding site (Batch Seq-2) (or a complementary sequence thereof), and (iii) the universal binding site for an amplification primer (universal RCA) (or a complementary sequence thereof). In some embodiments, the unit further comprises the universal compaction oligonucleotide binding site (or a complementary sequence thereof).

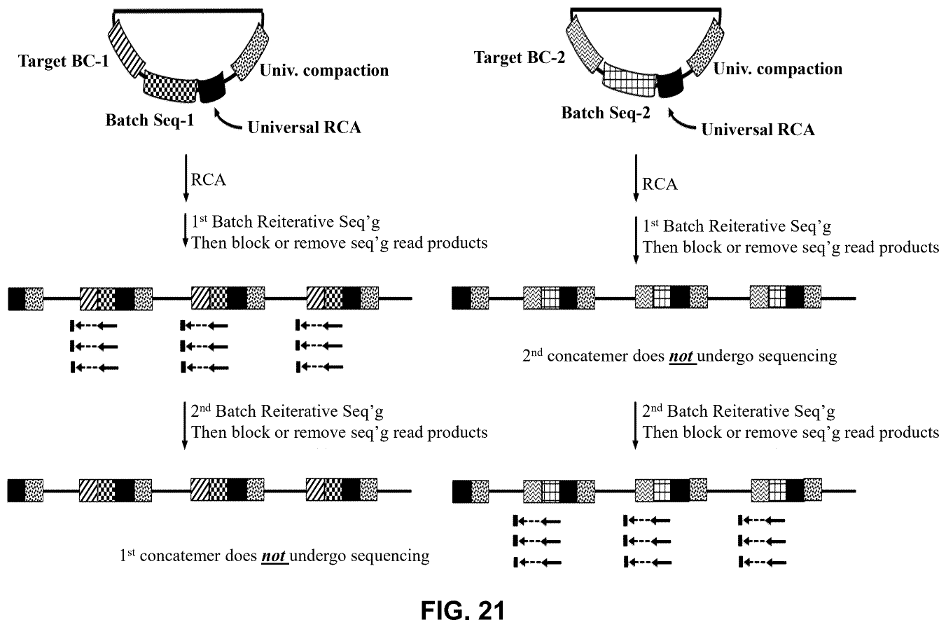

In some embodiments, the methods further comprise step (d): sequencing the first plurality of concatemer molecules inside the cellular sample under a condition that inhibits sequencing the second plurality of concatemers (e.g., FIG. 21). In some embodiments, step (d) comprises sequencing the first plurality of concatemers inside the cellular sample comprises conducting 2-1000 sequencing cycles to generate a plurality of first sequencing read products, wherein the sequences of the first sequencing read products are aligned with a first target reference sequence to confirm the presence of the first target RNA in the cellular sample. In some embodiments, step (d) comprises sequencing the first plurality of concatemers inside the cellular sample comprises conducting 1-250 sequencing cycles to generate a plurality of first sequencing read products, wherein the sequences of the first sequencing read products are aligned with a first target reference sequence to confirm the presence of the first target RNA in the cellular sample.

In some embodiments in step (d), in the first concatemer molecules, only the first target barcode region (target BC-1) is sequenced. In some embodiments, in the first concatemer molecules, at least a portion or the full length of the first target barcode (target BC-1) is sequenced. In some embodiments, in the first concatemer molecules, the first target barcode (target BC-1) is sequenced and a portion of the first cDNA region is sequenced.

In some embodiments, the sequencing the first concatemers of step (d) comprises step (1) contacting the first plurality of concatemer molecules inside the cellular sample with (i) a plurality of first batch-specific sequencing primers, (ii) a plurality of sequencing polymerases, and (iii) a plurality of nucleotide reagents, under a condition suitable for hybridizing the plurality of first batch-specific sequencing primers to their respective first batch-specific sequencing primer binding sites on the first concatemers. In some embodiments, the sequencing further comprises step (2) conducting 2-1000 sequencing cycles to generate a first plurality of sequencing read products using the first concatemers as template molecules.

In some embodiments, the sequencing of step (d) comprises sequencing at least a portion of the first nucleic acid concatemers using an optical imaging system comprising a field-of-view (FOV) greater than 1.0 mm².

In some embodiments, in the sequencing of step (d), the plurality of first sequencing read products are detectable by imaging, and wherein the sequencing comprises decoding the plurality of first sequencing read products from the images obtained during the no more than 2-30 sequencing cycles, or from the images obtained during the 1-1000 sequence cycles.

In some embodiments, the methods further comprise step (e): removing the plurality of first sequencing read products from the first concatemer molecules and retaining the first concatemer molecules inside the cellular sample. In some embodiments, a 3′ blocking moiety can be added to the first sequencing read products to inhibit further sequencing reactions. For example, a nucleotide analog can be incorporated where the nucleotide analog inhibits incorporation of a subsequent nucleotide. Exemplary blocking nucleotide analogs include dideoxynucleotide or a nucleotide having a 2′ or 3′ chain terminating moiety.

In some embodiments, the methods further comprise step (f): reiteratively sequencing the plurality of first concatemers by repeating steps (d) and (e) at least once. In some embodiments, reiterative sequencing of step (f) is optional.

In some embodiments, the sequencing the first concatemers of step (f) comprises step (1) contacting the first plurality of concatemer molecules inside the cellular sample with (i) a plurality of first batch-specific sequencing primers, (ii) a plurality of sequencing polymerases, and (iii) a plurality of nucleotide reagents, under a condition suitable for hybridizing the plurality of first batch-specific sequencing primers to their respective first batch-specific sequencing primer binding sites on the first concatemers. In some embodiments, the sequencing further comprises step (2) conducting 2-1000 sequencing cycles to generate a first plurality of sequencing read products using the first concatemers as template molecules. In some embodiments, the sequencing further comprises step (3) removing the first plurality of sequencing read products from the first concatemers and retaining the plurality of first concatemers inside the cellular sample. In some embodiments, the sequencing further comprises step (4) repeating steps (1)-(3) at least once (e.g., FIG. 21). In some embodiments, step (4) comprises repeating steps (1)-(3) at least 2 times, at least 3 times, at least 4 times, at least 5 times, at least 6 times, at least 7 times, at least 8 times, at least 9 times, or at least 10 times. In some embodiments, step (4) comprises repeating steps (1)-(3) up to 10 times, up to 20 times, up to 30 time, up to 40 times, or up to 50 times.

In some embodiments, the reiterative sequencing of the first concatemers of step (f) can be conducting using a sequencing-by-binding procedure, labeled and/or non-labeled chain-terminating nucleotides, or multivalent molecules. Descriptions of these three sequencing methods is described below.

In some embodiments, the plurality of universal sequencing primers can be hybridized to concatemer template molecules with a hybridization reagent comprising an SSC buffer (e.g., 2× saline-sodium citrate) buffer with formamide (e.g., 10-20% formamide). The hybridization conditions comprise a temperature of about 20-30° C., for about 10-60 minutes.

In some embodiments, the plurality of sequencing read products can be removed from the concatemers and the plurality of concatemers can be retained inside the cellular sample using a de-hybridization reagent comprising an SSC buffer (e.g., saline-sodium citrate) buffer, with or without formamide, at a temperature that promotes nucleic acid denaturation such as for example 30-90° C.

In some embodiments, the methods further comprise step (g): sequencing the second plurality of concatemer molecules inside the cellular sample under a condition that inhibits sequencing the first plurality of concatemers (e.g., FIG. 21). In some embodiments, step (g) comprises sequencing the second plurality of concatemers inside the cellular sample comprises conducting 2-1000 sequencing cycles to generate a plurality of second sequencing read products, wherein the sequences of the second sequencing read products are aligned with a second target reference sequence to confirm the presence of the second target RNA in the cellular sample. In some embodiments, step (g) comprises sequencing the second plurality of concatemers inside the cellular sample comprises conducting 1-250 sequencing cycles to generate a plurality of second sequencing read products, wherein the sequences of the second sequencing read products are aligned with a second target reference sequence to confirm the presence of the second target RNA in the cellular sample.

In some embodiments in step (g), in the second concatemer molecules, only the second target barcode region (target BC-2) is sequenced. In some embodiments, in the second concatemer molecules, at least a portion or the full length of the second target barcode (target BC-2) is sequenced. In some embodiments, in the second concatemer molecules, the second target barcode (target BC-2) is sequenced and a portion of the second cDNA region is sequenced.

In some embodiments, the sequencing the second concatemers of step (g) comprises step (1) contacting the second plurality of concatemer molecules inside the cellular sample with (i) a plurality of second batch-specific sequencing primers, (ii) a plurality of sequencing polymerases, and (iii) a plurality of nucleotide reagents, under a condition suitable for hybridizing the plurality of second batch-specific sequencing primers to their respective second batch-specific sequencing primer binding sites on the second concatemers. In some embodiments, the sequencing further comprises step (2) conducting 2-1000 sequencing cycles to generate a second plurality of sequencing read products using the second concatemers as template molecules.

In some embodiments, the sequencing of step (g) comprises sequencing at least a portion of the second nucleic acid concatemers using an optical imaging system comprising a field-of-view (FOV) greater than 1.0 mm².

In some embodiments, in the sequencing of step (g), the plurality of second sequencing read products are detectable by imaging, and wherein the sequencing comprises decoding the plurality of second sequencing read products from the images obtained during the no more than 2-30 sequencing cycles, or from the images obtained during the 1-1000 sequencing cycles.

In some embodiments, the methods further comprise step (h): removing the plurality of second sequencing read products from the second concatemer molecules and retaining the second concatemer molecules inside the cellular sample. In some embodiments, a 3′ blocking moiety can be added to the second sequencing read products to inhibit further sequencing reactions. For example, a nucleotide analog can be incorporated where the nucleotide analog inhibits incorporation of a subsequent nucleotide. Exemplary blocking nucleotide analogs include dideoxynucleotide or a nucleotide having a 2′ or 3′ chain terminating moiety.

In some embodiments, the methods further comprise step (i): reiteratively sequencing the plurality of second concatemers by repeating steps (g) and (h) at least once. In some embodiments, reiterative sequencing of step (i) is optional.

In some embodiments, the sequencing the second concatemers of step (i) comprises step (1) contacting the second plurality of concatemer molecules inside the cellular sample with (i) a plurality of second batch-specific sequencing primers, (ii) a plurality of sequencing polymerases, and (iii) a plurality of nucleotide reagents, under a condition suitable for hybridizing the plurality of second batch-specific sequencing primers to their respective second batch-specific sequencing primer binding sites on the second concatemers. In some embodiments, the sequencing further comprises step (2) conducting 2-1000 sequencing cycles to generate a first plurality of sequencing read products using the second concatemers as template molecules. In some embodiments, the sequencing further comprises step (3) removing the first plurality of sequencing read products from the second concatemers and retaining the plurality of second concatemers inside the cellular sample. In some embodiments, the sequencing further comprises step (4) repeating steps (1)-(3) at least once (e.g., FIG. 21). In some embodiments, step (4) comprises repeating steps (1)-(3) at least 2 times, at least 3 times, at least 4 times, at least 5 times, at least 6 times, at least 7 times, at least 8 times, at least 9 times, or at least 10 times. In some embodiments, step (4) comprises repeating steps (1)-(3) up to 10 times, up to 20 times, up to 30 time, up to 40 times, or up to 50 times.

In some embodiments, the reiterative sequencing of the second concatemers of step (i) can be conducting using a sequencing-by-binding procedure, labeled and/or non-labeled chain-terminating nucleotides, or multivalent molecules. Descriptions of these three sequencing methods is described below.

In some embodiments, the plurality of nucleotide reagents of steps (d) and (g) comprise a plurality of nucleotides that are detectably labeled or non-labeled. In some embodiments, individual nucleotides are linked to a detectable reporter moiety. In some embodiments, the detectable reporter moiety comprises a fluorophore. In some embodiments, the plurality of detectably labeled nucleotide analogs comprise a plurality of chain terminating nucleotides, where the chain terminating moiety is linked to 3′ nucleotide sugar position to form a 3′ blocked nucleotide analog. In some embodiments, the chain terminating moiety can be removed to convert 3′ blocked nucleotide analog to an extendible nucleotide having a 3′ OH group on the sugar. In some embodiments, the labeled nucleotide analogs are linked to a different fluorophore that corresponds to the nucleo-bases adenine, cytosine, guanine, thymine or uracil, where the different fluorophores emit a fluorescent signal. In some embodiments, a sequencing cycle comprises (1) contacting the concatemer/sequencing primer duplex with a sequencing polymerase and a detectably labeled chain terminating nucleotide under a condition suitable for polymerase-catalyzed incorporation of the detectably labeled chain terminating nucleotide into the terminal end of the sequencing primer, (2) detecting and imaging the fluorescent signal and color emitted by the incorporated chain terminating nucleotide, and (3) removing the chain terminating moiety (e.g., unblocking) and the fluorophore from the incorporated nucleotide and retaining the concatemer/sequencing primer duplex. In some embodiments, no more than 2-30 or no more than 1000 sequencing cycles are conducted on the plurality of concatemers inside the cellular sample to generate a plurality of sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the second target RNA molecules inside the cellular sample.

In some embodiments, the sequences of the first and second sequencing read products can be aligned after each round of generating the first and second sequencing read products which are no more than 30 to 150 bases in length, or after generating a set of reiterative sequencing read products wherein the first and second sequencing read products which are no more than 30 to 150 bases in length. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules. In some embodiments, when the sequencing is conducted using different fluorescently-labeled nucleotide reagents that correspond to different nucleo-bases (e.g., A, G, C, T/U), then the images can have different color fluorescent spots co-located in the same cellular sample at different sequencing cycles.

In some embodiments, out-of-sync phasing and/or pre-phasing events can occur during synchronized sequencing reactions on clonally amplified template amplicons, where the sequencing reactions comprise polymerase-catalyzed sequencing reactions employing detectably labeled chain terminator nucleotides. In some embodiments, a sequencing reaction on one template molecule in the clonally-amplified template molecules moves ahead (e.g., pre-phasing) or fall behind (e.g., phasing) of the sequencing of the other template molecules within the clonally-amplified template molecules. During sequencing, a fluorescent signal is typically detected which corresponds to incorporation of a labeled chain terminator nucleotide. Thus, phasing and pre-phasing events can be detected and monitored using incorporation of a labeled chain terminator nucleotide.

In some embodiments, the plurality of nucleotide reagents of steps (d) and (g) comprise a plurality of multivalent molecules each comprising a core attached to a plurality of nucleotide-arms, wherein the nucleotide-arms are attached to a nucleotide unit. In some embodiments, individual multivalent molecules are labeled with a detectably reporter moiety. In some embodiments, the detectable reporter moiety comprises a fluorophore. In some embodiments, the core of the multivalent molecule is labeled with a fluorophore, and wherein the fluorophore which is attached to a given core of the multivalent molecule corresponds to the nucleotide base (e.g., adenine, guanine, cytosine, thymine or uracil) of the nucleotide arm. In some embodiments, at least one of the nucleotide arms of the multivalent molecule comprises a linker and/or nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments, a sequencing cycle comprises (1) contacting the concatemer/sequencing primer duplex with a first sequencing polymerase to form a complexed polymerase, (2) contacting the complexed polymerase with a detectably labeled multivalent molecule under a condition suitable for binding a complementary nucleotide unit of the multivalent molecule to the complexed polymerase thereby forming a multivalent-binding complex, and the condition is suitable for inhibiting incorporation of the complementary nucleotide unit into the terminal end of the sequencing primer, (3) detecting and imaging the fluorescent signal and color emitted by the bound detectably labeled multivalent molecule, (4) removing the first sequencing polymerase and the bound detectably labeled multivalent molecule, and retaining the concatemer/sequencing primer duplex, (5) contacting the retained concatemer/sequencing primer duplex with a second sequencing polymerase and a non-labeled chain terminating nucleotide under a condition suitable for polymerase-catalyzed incorporation of the non-labeled chain terminating nucleotide into the terminal end of the sequencing primer, and (6) removing the chain terminating moiety (e.g., unblocking) and retaining the concatemer/sequencing primer duplex. In some embodiments, no more than 2-30 or no more than 1000 sequencing cycles are conducted on the plurality of concatemers inside the cellular sample to generate a plurality of sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the second target RNA molecules inside the cellular sample. In some embodiments, the sequences of the first and second sequencing read products can be aligned after each round of generating the first and second sequencing read products which are no more than 30 or no more than 1000 bases in length, or after generating a set of reiterative sequencing read products wherein the first and second sequencing read products which are no more than 30 or no more than 1000 bases in length. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules. In some embodiments, individual cycle times can be achieved in less than 30 minutes. In some embodiments, the field of view (FOV) can exceed 1 mm² and the cycle time for scanning large area (>10 mm²) can be less than 5 minutes.

In any of the methods described herein, the plurality of RNA or cDNA inside the cellular sample can be amplified to generate amplicons of the RNA or cDNA where the amplicons comprise concatemers. In some embodiments, the plurality of RNA or cDNA molecules inside the cellular sample can be amplified by conducting a padlock probe circularization and rolling circle amplification workflow. In some embodiments, the methods comprise contacting the plurality of RNA or cDNA molecules inside the cellular sample with a plurality of padlock probes, including a first plurality of target-specific padlock probes that hybridize with first target RNA or cDNA molecules, and a second plurality of target-specific padlock probes that hybridize with second target RNA or cDNA molecules.

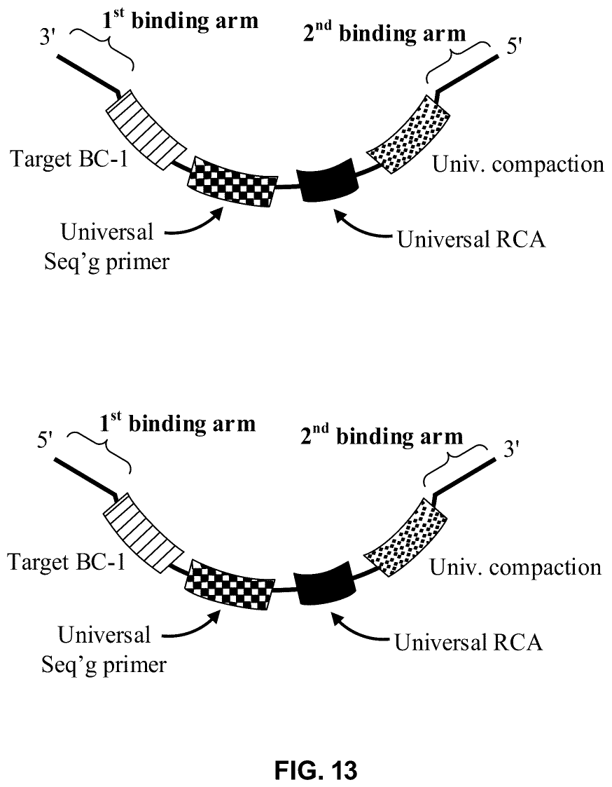

In some embodiments, the padlock probes comprise single-stranded oligonucleotides. In some embodiments, the padlock probes comprise DNA, RNA, or DNA and RNA. In some embodiments, individual padlock probes comprise an internal region between the first and second terminal regions, where the internal region comprises at least one universal adaptor sequence including a sample barcode sequence, an amplification primer binding site, a sequencing primer binding site, a compaction oligonucleotide binding site and/or a surface capture primer binding site (FIG. 13). In some embodiments, the padlock probes comprise at least one target barcode sequence that corresponds to a given target RNA or target cDNA to which the padlock probes binds. In some embodiments, the padlock probes comprise at least one unique identification sequence (e.g., unique molecular index (UMI)). In some embodiments, the padlock probes comprise at least one restriction enzyme recognition sequence.

In some embodiments, a padlock probe comprises a single-stranded nucleic acid molecule having two terminal regions (e.g., first and second binding arms) and an internal region. In some embodiments, the first terminal region of an individual padlock probe has a first target-specific sequence that selectively hybridizes to a first region of a target RNA or target cDNA molecule, and the second terminal region of the individual padlock probe has a second target-specific sequence that selectively hybridizes to a second region of the same target RNA or target cDNA molecule. In some embodiments, the internal region of a padlock comprises a target barcode sequence (e.g., Target BC-1 or Target BC-2, left and right schematics respectively) which corresponds to a given target RNA or target cDNA. In some embodiments, the target barcode sequence uniquely identifies the target RNA or target cDNA. In some embodiments, the internal region of a padlock comprises a universal primer binding site for a sequencing primer (or a complementary sequence thereof). In some embodiments, the internal region of a padlock comprises a universal primer binding site for a rolling circle amplification primer (or a complementary sequence thereof). In some embodiments, the internal region of a padlock comprises a universal binding site for a compaction oligonucleotide binding (or a complementary sequence thereof). In some embodiments, the internal region of a padlock probe includes a target barcode sequence and at least one universal primer binding site (e.g., for binding a sequencing primer, for binding a rolling circle amplification primer and/or for binding a compaction oligonucleotide) in any arrangement and orientation (FIG. 13, top and bottom).

In some embodiments, individual padlock probes comprise first and second terminal regions (e.g., first and second binding arms) that hybridize to portions of target RNA or target cDNA molecules to form a plurality of RNA-padlock probe complexes or a plurality of cDNA-padlock probe complexes, wherein individual complexes have the first and second terminal probe regions hybridized to proximal regions of an RNA or cDNA molecule to form a nick or gap between the first and second terminal probe ends. In some embodiments, the first terminal region of an individual padlock probe has a first target-specific sequence that selectively hybridizes to a first region of a target RNA or cDNA molecule, and the second terminal region of the individual padlock probe has a second target-specific sequence that selectively hybridizes to a second region of the same target RNA or cDNA molecule, where a nick or gap is formed between the hybridized first and second terminal regions, thereby circularizing the padlock probe (e.g., FIG. 14).

In some embodiments, the padlock probes comprise canonical nucleotides and/or nucleotide analogs. In some embodiments, the padlock probes are modified to confer resistance to nuclease degradation (e.g., ribonuclease degradation). For example, the padlock probes comprise at least one phosphorothioate diester bond at their 5′ ends which can render the padlock probes resistant to nuclease degradation. In some embodiments, the padlock probes comprise 2-5 or more consecutive phosphorothioate diester bonds at their 5′ ends. In some embodiments, the padlock probes comprise at least one ribonucleotide and/or at least one 2′-O-methyl, 2′-O-methoxyethyl (MOE), 2′ fluoro-base nucleotide. In some embodiments, the padlock probes comprise phosphorylated 3′ ends. In some embodiments, the padlock probes comprise at least one locked nucleic acid (LNA) base. In some embodiments, the padlock probes comprise a phosphorylated 5′ end (e.g., using a polynucleotide kinase).

In some embodiments, individual padlock probes in a set of padlock probes (e.g., a plurality of padlock probes) comprise first and second terminal regions that hybridize to the same target regions of the target RNA or cDNA molecules to form a plurality of RNA-padlock probe complexes or a plurality of cDNA-padlock probe complexes having the same RNA or cDNA sequence.

In some embodiments, a set of padlock probes (e.g., a plurality of padlock probes) comprise at least two sub-sets of padlock probes. In some embodiments, individual padlock probes in a first sub-set of padlock probes comprise first and second terminal regions that hybridize to the same target regions (e.g., a first target region) of the target RNA or cDNA molecules to form a first plurality of RNA-padlock probe complexes or a first plurality of cDNA-padlock probe complexes having the same RNA or cDNA sequence. In some embodiments, individual padlock probes in a second sub-set of padlock probes comprise first and second terminal regions that hybridize to the same target regions (e.g., a second target region) of the target RNA or cDNA molecules to form a second plurality of RNA-padlock probe complexes or a second plurality of cDNA-padlock probe complexes having the same cDNA sequence. In some embodiments, the first and second sub-sets of padlock probes hybridize to different target regions of the same target RNA or cDNA molecules. In some embodiments, the first and second sub-sets of padlock probes hybridize to different target regions of different target RNA or cDNA molecules. In some embodiments, the set of padlock probes comprise 2-10 sub-sets of padlock probes, or 10-25 sub-sets of padlock probes, or 25-50 sub-sets of padlock probes, or up to 100 sub-sets of padlock probes. In some embodiments, the set of padlock probes comprise at least 100 sub-sets of padlock probes, at least 500 sub-sets of padlock probes, at least 1000 sub-sets of padlock probes, at least 10,000 sub-sets of padlock probes, or more sub-sets of padlock probes.

In some embodiments, the nicks can be enzymatically ligated to generate covalently closed circular padlock probes. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

In some embodiments, the size of the gap between the hybridized first and second terminal regions is 1-25 bases. The 3′OH end of hybridized padlock probe can serve as an initiation site for a polymerase-catalyzed fill-in reaction (e.g., gap fill-in reaction) using the target cDNA molecule (or the target RNA molecule) as a template. After the fill-in reaction, the remaining nick can be enzymatically ligated to generate covalently closed circular padlock probes.

In some embodiments, the gap-filling reaction comprises contacting the circularized padlock probe with a DNA polymerase and a plurality of nucleotides. In some embodiments, the DNA polymerase comprises E. coli DNA polymerase I, Klenow fragment of E. coli DNA polymerase I, T7 DNA polymerase, or T4 DNA polymerase. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

In any of the methods described herein, the plurality of covalently closed circular padlock probes can be subjected to a rolling circle amplification reaction to generate a plurality of concatemer molecules each having two or more tandem copies of a unit wherein the unit comprises a target sequence that corresponds to a target RNA molecules and any additional sequence(s) carried by the padlock probes including universal adaptor sequence(s), unique molecular index sequence(s) and/or restriction enzyme recognition sequence(s).

In some embodiments, the rolling circle amplification reaction comprises contacting the covalently closed circularized padlock probes with an amplification primer (e.g., a universal rolling circle amplification primer), a strand-displacing DNA polymerase, and a plurality of nucleotides, under a condition suitable for hybridizing individual amplification primers to a covalently closed padlock probe, and under a condition suitable for conducting primer extension using the covalently closed padlock probe as a template molecule to generate a nucleic acid concatemer. In some embodiments, the plurality of nucleotides in the rolling circle amplification reaction comprise any mixture of two or more of dATP, dGTP, dCTP, dTTP and/or dUTP. In some embodiments, any of the rolling circle amplification reactions described herein can be conducted in the presence or in the absence of a plurality of compaction oligonucleotides.

In some embodiments, when the rolling circle amplification reaction includes a plurality of nucleotide which includes dUTP, the resulting concatemer can be cross-linked to a cross-linking reactive group by treating the cellular sample with a succinimide ester (NHS), maleimide (Sulfo-SMCC), imidoester (DMP), carbodiimide (DCC, EDC) or phenyl azide. In some embodiments, polymerization of the cross-linking reactive group can be initiated with light or UV light. In some embodiments, the resulting concatemer can be cross-linked to a matrix by treating the cellular sample with a cross-linked agarose, cross-linked dextran or cross-linked polyethylene glycol (PEG), polyacrylamide, cellulose alginate or polyamide. In some embodiments, the PEG comprises a sulfo-NHS ester moiety at one or both ends, for example a PEGylated bis(sulfosuccinimidyl) suberate) (e.g., BS(PEG)₉ from Thermo Fisher Scientific, catalog No. 21582).

In some embodiments, the rolling circle amplification reaction can be conducted at a constant temperature (e.g., isothermal) wherein the constant temperature is at room temperature to about 30° C., or about 30-40° C., or about 40-50° C., or about 50-65° C.

In some embodiments, the DNA polymerase having a strand displacing activity can be selected from a group consisting of phi29 DNA polymerase, large fragment of Bst DNA polymerase, large fragment of Bsu DNA polymerase, and Bca (exo-) DNA polymerase, Klenow fragment of E. coli DNA polymerase, T5 polymerase, M-MuLV reverse transcriptase, HIV viral reverse transcriptase, or Deep Vent DNA polymerase. In some embodiments, the phi29 DNA polymerase can be wild type phi29 DNA polymerase (e.g., MagniPhi from Expedeon), or variant EquiPhi29 DNA polymerase (e.g., from Thermo Fisher Scientific), and chimeric QualiPhi DNA polymerase (e.g., from 4basebio).

In some embodiments, the rolling circle amplification primers can be modified to increase resistance to nuclease degradation. In some embodiments, the rolling circle amplification primers comprise at least one phosphorothioate diester bond at their 5′ ends which can render the amplification primers resistant to exonuclease degradation. In some embodiments, the rolling circle amplification primers comprise 2-5 or more consecutive phosphorothioate diester bonds at their 5′ ends. In some embodiments, the rolling circle amplification primers comprise at least one ribonucleotide and/or at least one 2′-O-methyl or 2′-O-methoxyethyl (MOE) nucleotide.

In some embodiments, the rolling circle amplification reaction can be conducted in the presence of a plurality of compaction oligonucleotides which, when hybridized to a concatemer molecule, compacts the size and/or shape of the concatemer to form a compact nanoball. In some embodiments, the compaction oligonucleotides comprise single stranded oligonucleotides having a first region at one end that hybridizes to a portion of a concatemer molecule and a second region at the other end that hybridizes to another portion of the same concatemer molecule, where hybridization of the compaction oligonucleotide to a given concatemer compacts the size and/or shape of the concatemer.

The compaction oligonucleotides include a 5′ region, an optional internal region (intervening region), and a 3′ region. The 5′ and 3′ regions of the compaction oligonucleotide can hybridize to any portions of the concatemer. The 5′ and 3′ regions of the compaction oligonucleotide can hybridize to different portions of the concatemer to pull together distal portions of the concatemer causing compaction of the concatemer to form a DNA nanoball. For example, 5′ region of the compaction oligonucleotide is designed to hybridize to a first portion of the concatemer molecule (e.g., a universal compaction oligonucleotide binding site), and 3′ region of the compaction oligonucleotide is designed to hybridized to a second portion of the concatemer molecule (e.g., a universal compaction oligonucleotide binding site). Inclusion of compaction oligonucleotides during RCA can promote formation of DNA nanoballs having tighter size and shape compared to concatemers generated in the absence of the compaction oligonucleotides. The compact and stable characteristics of the DNA nanoballs improves in situ sequencing accuracy by increasing signal intensity and the nanoballs retain their shape and size during multiple sequencing cycles.

In some embodiments, the compaction oligonucleotides comprise single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprises a 5′ region and a 3′ region, and optionally an intervening region between 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides can be wholly complementary or partially complementary along its length to a first portion of a concatemer molecule. The 3′ region of the compaction oligonucleotides can be wholly complementary or partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule.

In some embodiments, 5′ region of the compaction oligonucleotide can have the same sequence as 3′ region. The 5′ region of the compaction oligonucleotide can have a sequence that is different from 3′ region. In some embodiments, 3′ region of the compaction oligonucleotide can have a sequence that is a reverse sequence of 5′ region. In some embodiments, 5′ region of the compaction oligonucleotide can have a sequence that is a reverse sequence of 3′ region.

In some embodiments, 3′ region of any of the compaction oligonucleotides can include an additional three bases at the terminal 3′ end which comprises 2′-O-methyl RNA bases (e.g., designated mUmUmU) or the terminal 3′ end lacks additional 2′-O-methyl RNA bases.

In some embodiments, the compaction oligonucleotides comprise one or more modified bases or linkages at their 5′ or 3′ ends to confer certain functionalities. In some embodiments, the compaction oligonucleotides comprise at least one phosphorothioate linkages at their 5′ and/or 3′ ends to confer exonuclease resistance. In some embodiments, at least one nucleotide at or near 3′ end comprises a 2′ fluoro base which confers exonuclease resistance. In some embodiments, 3′ end of the compaction oligonucleotides comprise at least one 2′-O-methyl RNA base which blocks polymerase-catalyzed extension. For example, 3′ end of the compaction oligonucleotide comprises three bases comprising 2′-O-methyl RNA base (e.g., designated mUmUmU). In some embodiments, the compaction oligonucleotides comprise a 3′ inverted dT at their 3′ ends which blocks polymerase-catalyzed extension. In some embodiments, the compaction oligonucleotides comprise 3′ phosphorylation which blocks polymerase-catalyzed extension. In some embodiments, the internal region of the compaction oligonucleotides comprise at least one locked nucleic acid (LNA) which increases the thermal stability of duplexes formed by hybridizing a compaction oligonucleotide to a concatemer molecule. In some embodiments, the compaction oligonucleotides comprise a phosphorylated 5′ end (e.g., using a polynucleotide kinase).

In some embodiments, the compaction oligonucleotide comprises the sequence 5′-CATGTAATGCACGTACTTTCAGGGTAAACATGTAATGCACGTACTTT CAGGGT-3′ (SEQ ID NO: 1). In some embodiments, the compaction oligonucleotides includes an additional three bases at the terminal 3′ end which comprises 2′-O-methyl RNA bases (e.g., designated mUmUmU) or the terminal 3′ end lacks additional 2′-O-methyl RNA bases.

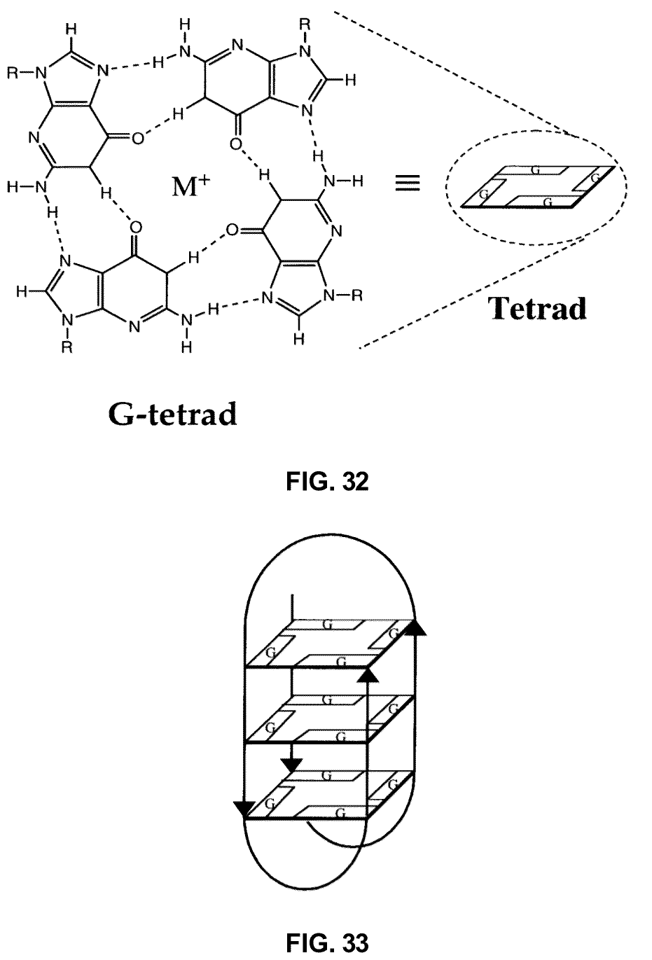

In some embodiments, the compaction oligonucleotides can include at least one region having consecutive guanines. For example, the compaction oligonucleotides can include at least one region having 2, 3, 4, 5, 6 or more consecutive guanines. In some embodiments, the compaction oligonucleotides comprise four consecutive guanines which can form a guanine tetrad structure (see FIG. 32). The guanine tetrad structure can be stabilized via Hoogsteen hydrogen bonding. The guanine tetrad structure can be stabilized by a central cation including potassium, sodium, lithium, rubidium or cesium.

At least one compaction oligonucleotide can form a guanine tetrad (FIG. 32) and hybridize to the universal binding sequences in a concatemer which can cause the concatemer to fold to form an intramolecular G-quadruplex structure (FIG. 33). The concatemers can self-collapse to form compact nanoballs. Formation of the guanine tetrads and G-quadruplexes in the nanoballs may increase the stability of the nanoballs to retain their compact size and shape which can withstand changes in pH, temperature and/or repeated flows of reagents during sequencing inside the cellular sample.

In some embodiments, the plurality of compaction oligonucleotides in the rolling circle amplification reaction have the same sequence. Alternatively, the plurality of compaction oligonucleotides in the rolling circle amplification reaction comprise a mixture of two or more different populations of compaction oligonucleotides having different sequences.

In some embodiment, the immobilized concatemer template molecule can self-collapse into a compact nucleic acid nanoball. The nanoballs can be imaged and a FWHM measurement can be obtained to give the shape/size of the nanoballs.

In some embodiments, inclusion of compaction oligonucleotides in the rolling circle amplification reaction can promote collapsing of a concatemer into a DNA nanoball. Conducting RCA with compaction oligonucleotides helps retain the compact size and shape of a DNA nanoball during multiple sequencing cycles which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

The single-stranded concatemers collapse into compact DNA nanoballs, where each nanoball carries numerous tandem copies of a polynucleotide unit along their lengths, where the polynucleotide unit includes a sequence-of-interest (e.g., that corresponds to target RNA or target cDNA) and at least a universal sequencing primer binding site. Each polynucleotide unit can bind a sequencing primer, a sequencing polymerase and a detectably-labeled nucleotide reagent (e.g., detectably labeled multivalent molecules), to form a detectable sequencing complex (e.g., a detectable ternary complex). Each nanoball carries numerous detectable sequencing complexes. Thus, the compact nature of the nanoballs increases the local concentration of detectably-labeled nucleotide reagents that are used during the sequencing workflow which increases the signal intensity emitted from a nanoball to give a discrete detectable signal which can be imaged as a fluorescent spot inside the cellular sample. Each spot corresponds to a concatemer and each concatemer corresponds to a target RNA molecule in the cellular sample. Multiple spots can be detected and imaged simultaneously in the cellular sample. The DNA nanoballs having compact shape and size that produce increased signal intensity and color differentiation during sequencing.

### Cellular Samples

In any of the methods described herein, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, or an FFPE cellular sample. In some embodiments, the cellular sample comprise one or more living cells or non-living cells.

In some embodiments, the cellular sample can be obtained from a virus, fungus, prokaryote or eukaryote. In some embodiments, the cellular sample can be obtained from an animal, insect or plant. In some embodiments, the cellular sample comprises one or more virally-infected cells.

In some embodiments, the cellular sample can be obtained from any organism including human, simian, ape, canine, feline, bovine, equine, murine, porcine, caprine, lupine, ranine, piscine, plant, insect or bacteria.

In some embodiments, the cellular sample can be obtained from any organ including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs.

In any of the methods described herein, the cellular sample harbors a plurality of RNA which include target RNA and non-target RNA. In some embodiments, cells typically produce RNA by gene expression which includes transcription of DNA (e.g., genomic DNA) into RNA molecules. The transcribed RNA can undergo splicing or may not be spliced. The transcribed RNA can be translated into a polypeptide (e.g., coding RNA), or do not undergo translation but can be processed into tRNA or rRNA (e.g., non-coding RNA).

In some embodiments, the plurality of RNA harbored by the cellular sample includes target and non-target RNA. In some embodiments, the plurality of RNA harbored by the cellular sample comprises wild type RNA, mutant RNA or splice variant RNA. In some embodiments, the plurality of RNA harbored by the cellular sample comprises pre-spliced RNA, partially spliced RNA, or fully spliced RNA. In some embodiments, the plurality of RNA harbored by the cellular sample comprises coding RNA, non-coding RNA, mRNA, tRNA, rRNA, microRNA (miRNA), mature microRNA, or immature microRNA. In some embodiments, the plurality of RNA harbored by the cellular sample comprises housekeeping RNA, cell-specific RNA, tissue-specific RNA or disease-specific RNA. In some embodiments, the plurality of RNA harbored by the cellular sample comprises RNA expressed by one or more cells in response to a stimulus such as heat, light, a chemical or a drug. In some embodiments, the plurality of RNA harbored by the cellular sample comprises RNA found in healthy cells or diseased cells. In some embodiments, the plurality of RNA harbored by the cellular sample comprises RNA transcribed from transgenic DNA sequences that are introduced into the cellular sample using recombinant DNA procedures. For example, the RNA can be transcribed from a transgenic DNA sequence that is controlled by an inducible or constitutive promoter sequence. In some embodiments, the plurality of RNA harbored by the cellular sample comprises RNA that is transcribed from DNA sequences that are not transgenic.

In any of the methods described herein, the cellular sample can be cultured on the support. In some embodiments, the methods comprise culturing the cellular sample on the support under a condition suitable for expanding the cellular sample for 2-10 generations or more. The cultured cellular sample can generate a colony of cells. In some embodiments, the methods comprise culturing the cellular sample to confluence or non-confluence. In some embodiments, the methods comprise culturing the cellular sample on the support in a simple or complex cell culture media. For example, the cell culture media comprises D-MEM high glucose (e.g., from Thermo Fisher Scientific, catalog No. 11965118), fetal bovine serum (e.g., 10% FBS; for example from Thermo Fisher Scientific, catalog No. A3160402), MEM non-essential amino acids (e.g., 0.1 mM MEM, for example from Thermo Fisher Scientific, catalog No. 11140050), L-glutamine (e.g., 6 mM L-glutamine, for example from Thermo Fisher Scientific, catalog No. A2916801), MEM sodium pyruvate (e.g., 1 mM sodium pyruvate, for example from Thermo Fisher Scientific, catalog No. 11360070), and an antibiotic (e.g., 1% penicillin-streptomycin-glutamine, for example from Thermo Fisher, catalog No. 10378016). In some embodiments, the methods comprise culturing the cellular sample at a humidity and temperature that is suitable for culturing the cell(s) on the support. Exemplary suitable conditions comprise approximately 37° C. with a humidified atmosphere of approximately 5-10% carbon dioxide in air. The cellular sample can be cultured with suitable aeration with oxygen and/or nitrogen.

In any of the methods described herein, the term “simple cell media” or related terms refers to a cell media that typically lacks ingredients to support cell growth and/or proliferation in culture. Simple cell media can be used for example to wash, suspend, or dilute the cellular sample. Simple cell media can be mixed with certain ingredients to prepare a cell media that can support cell growth and/or proliferation in culture. A simple cell media comprises any one or any combination of two or more of a buffer, a phosphate compound, a sodium compound, a potassium compound, a calcium compound, a magnesium compound and/or glucose. In some embodiments, the simple cell media comprises PBS (phosphate buffered saline), DPBS (Dulbecco's phosphate-buffered saline), HBSS (Hank's balanced salt solution), DMEM (Dulbecco's Modified Eagle's Medium), EMEM (Eagle's Minimum Essential Medium), and/or EBSS. In some embodiments, the cellular sample can be placed in a simple cell media prior to or during the step of conducting any of the nucleic acid methods described herein.

In any of the methods described herein, the term “complex cell media” or related terms refers to a cell media that can be used to support cell growth and/or proliferation in culture without supplementation or additives. Complex cell media can include any combination of two or more of a buffering system (e.g., HEPES), inorganic salt(s), amino acid(s), protein(s), polypeptide(s), carbohydrate(s), fatty acid(s), lipid(s), purine(s) and their derivatives (e.g., hypoxanthine), pyrimidine(s) and their derivatives, and/or trace element(s). Complex cell media includes fluids obtained from a fluid or tissue extract. Complex cell media includes artificial cell media. In some embodiments, complex cell media can be a serum-containing media, for example complex cell media includes fluids such as fetal bovine serum, blood plasma, blood serum, lymph fluid, human placental cord serum and amniotic fluid. In some embodiments, complex cell media can be a serum-free media, which are typically (but not necessarily) defined cell culture media. In some embodiments, complex cell media can be a chemically-defined media which typically (but not necessarily) include recombinant polypeptides, and ultra-pure inorganic and/or organic compounds. In some embodiments, complex cell media can be a protein-free media which include for example MEM (minimal essential media) and RPMI-1640 (Roswell Park Memorial Institute). In some embodiments, the complex cell media comprises IMDM (Iscove's Modified Dulbecco's Medium. In some embodiments, the complex cell media comprises DMEM (Dulbecco's Modified Eagle's Medium). In some embodiments, the cellular sample can be placed in a complex cell media prior to or during the step of conducting any of the nucleic acid methods described herein.

In any of the methods described herein, the cellular sample comprises a fixed cellular sample. In some embodiments, the cellular sample can be treated with a fixation reagent (e.g., a fixing reagent) that preserves the cell and its contents to inhibit degradation and can inhibit cell lysis. For example, the fixation reagent can preserve RNA harbored by the cellular sample. In some embodiments, the fixation reagent inhibits loss of nucleic acids from the cellular sample.

In some embodiments, the fixation reagent can cross-link the RNA to prevent the RNA from escaping the cellular sample. In some embodiments, a cross-linking fixation reagent comprises any combination of an aldehyde, formaldehyde, paraformaldehyde, formalin, glutaraldehyde, imidoesters, N-hydroxysuccinimide esters (NHS) and/or glyoxal (a bifunctional aldehyde).

In some embodiments, the fixation reagent comprises at least one alcohol, including methanol or ethanol. In some embodiments, the fixation reagent comprises at least one ketone, including acetone. In some embodiments, the fixation reagent comprises acetic acid, glacial acetic acid and/or picric acid. In some embodiments, the fixation reagent comprises mercuric chloride. In some embodiments, the fixation reagent comprises a zinc salt comprising zinc sulphate or zinc chloride. In some embodiments, the fixation reagent can denature polypeptides.

In some embodiments, the fixation reagent comprises 4% w/v of paraformaldehyde to water/PBS. In some embodiments, the fixation reagent comprises 10% of 35% formaldehyde at a neutral pH. In some embodiments, the fixation reagent comprises 2% v/v of glutaraldehyde to water/PBS. In some embodiments, the fixation reagent comprises 25% of 37% formaldehyde solution, 70% picric acid and 5% acetic acid.

In some embodiments, the cellular sample can be fixed on the support with 4% paraformaldehyde for about 30-60 minutes and washed with PBS.

In some embodiments, the cellular sample can be stained, de-stained or un-stained.

In any of the methods described herein, the cellular sample comprises a permeabilized cellular sample. In some embodiments, the methods comprise treating the cellular sample with a permeabilization reagent that alters the cell membrane to permit penetration of experimental reagents into the cells. For example, the permeabilization reagent removes membrane lipids from the cell membrane. In some embodiments, the cellular sample can be treated with a permeabilization reagent which comprises any combination of an organic solvent, detergent, chemical compound, cross-linking agent and/or enzyme. In some embodiments, the organic solvents comprise acetone, ethanol, and methanol. In some embodiments, the detergents comprise saponin, Triton X-100, Tween-20, sodium dodecyl sulfate (SDS), an N-lauroylsarcosine sodium salt solution, or a nonionic polyoxyethylene surfactant (e.g., NP40). In some embodiments, the cross-linking agent comprises paraformaldehyde. In some embodiments, the enzyme comprises trypsin, pepsin or protease (e.g. proteinase K). In some embodiments, the cells can be permeabilized using an alkaline condition, or an acidic condition with a protease enzyme. In some embodiments, the permeabilization reagent comprises water and/or PBS.

For example, the fixed cells can be permeabilized with 70% ethanol for about 30-60 minutes, and the permeabilizing reagent can be exchanged with PBS-T (e.g., PBS with 0.05% Tween-20). In some embodiments, the cells can be post-fixed with 3% paraformaldehyde and 0.1% glutaraldehyde for about 30-60 minutes, and washed with PBS-T multiple times.

In any of the methods described herein, the cellular sample is infused with a swellable polyelectrolyte hydrogel (U.S. Pat. No. 10,309,879 and Chen 2015 Science 347:543, the contents of these documents are incorporated by reference in their entireties). In some embodiments, a fixed and permeabilized cellular sample can be infused with sodium acrylate, acrylamide and a cross-linker N—N′-methylenebisacrylamide. In some embodiments, ammonium persulfate (APS) initiator and tetramethylethylenediamine (TEMED) accelerator were infused to achieve polymerization. In some embodiments, the cellular sample can be infused with proteinase K for proteolysis and incubated in a digestion buffer. In some embodiments, the gel inside the cellular sample can be swelled by addition of water.

In any of the methods described herein, the plurality of RNAs inside cellular sample can be converted to cDNA. In some embodiments, the methods comprise contacting the plurality of RNA inside the fixed and permeabilized cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample. In some embodiments, synthesis of second strand cDNA molecules is omitted. In some embodiments, the RNA inside the cellular sample is not converted into cDNA, where the RNA is hybridized to target-specific padlock probes.

In some embodiments, the reverse transcriptase enzyme exhibits RNA-dependent DNA polymerase activity. In some embodiments, the reverse transcriptase enzyme comprises a reverse transcriptase enzyme from AMV (avian myeloblastosis virus), M-MuLV (moloney murine leukemia virus), or HIV (human immunodeficiency virus). In some embodiment, the reverse transcriptase enzyme comprises a recombinant enzyme that exhibits reduced RNase H activity, for example REVERTAID (e.g., from Thermo Fisher Scientific, catalog No. EP0441). In some embodiments, the reverse transcriptase can be a commercially-available enzyme, including MULTISCRIBE (e.g., from Thermo Fisher Scientific, catalog #4311235), THERMOSCRIPT (e.g., from Thermo Fisher Scientific, catalog #12236-014), or ARRAYSCRIPT (e.g., from Ambion, catalog No. AM2048). In some embodiments, the reverse transcriptase enzyme comprises SUPERSCRIPT II (e.g., catalog No. 18064014), SUPERSCRIPT III (e.g., catalog No. 18080044), or SUPERSCRIPT IV enzymes (e.g., catalog No. 18090010) (all SUPERSCRIPT enzymes from Invitrogen). In some embodiments, the reverse transcription reaction can include an RNase inhibitor.

In some embodiments, the reverse transcription primers comprise a single-stranded oligonucleotide comprising DNA, RNA, or chimeric DNA/RNA. In some embodiments, the reverse transcription primers Any combination of adenine (A), thymine (T), guanine (G), cytosine (C), uracil (U) and/or inosine (I). In some embodiments, the reverse transcription primers can be any length, for example 5-25 bases, or 25-50 bases, or 50-75 bases, or 75-100 bases in length or longer. The reverse transcription primers each comprise a 5′ end and 3′ end. In some embodiments, 3′ end of the reverse transcription primers can include a 3′ OH moiety which serves as a nucleotide polymerization initiation site in a polymerase-catalyzed primer extension reaction. In some embodiments, 3′ end of the reverse transcription primers have a chain terminating moiety which blocks a polymerase-catalyzed primer extension reaction. The chain terminating moiety can be removed to convert 3′ sugar position to an extendible 3′OH.

In some embodiments, the reverse transcription primers are modified to confer resistance to nuclease degradation (e.g., ribonuclease degradation). For example, the reverse transcription primers comprise at least one phosphorothioate diester bond at their 5′ ends which can render the reverse transcription primers resistant to nuclease degradation. In some embodiments, the reverse transcription primers comprise 2-5 or more consecutive phosphorothioate diester bonds at their 5′ ends. In some embodiments, the plurality of reverse transcription primers comprise at least one ribonucleotide and/or at least one 2′-O-methyl, 2′-O-methoxyethyl (MOE), 2′ fluoro-base nucleotide. In some embodiments, the reverse transcription primers comprise phosphorylated 3′ ends. In some embodiments, the reverse transcription primers comprise locked nucleic acid (LNA) bases. In some embodiments, the reverse transcription primers comprise a phosphorylated 5′ end (e.g., using a polynucleotide kinase).

In some embodiments, the entire length of a reverse transcription primer can hybridize to a portion of an RNA molecule. In some embodiments, individual reverse transcription primers comprise a 3′ region having a sequence that hybridizes to a portion of an RNA molecule and a 5′ region that carries a tail that does not hybridize to an RNA molecule. In some embodiments, 5′ tail comprises a universal adaptor sequence including any one or any combination of two or more of a sample barcode sequence, an amplification primer binding site, a sequencing primer binding site, a compaction oligonucleotide binding site and/or a surface capture primer binding site. In some embodiments, 5′ tail comprises a unique identification sequence (e.g., unique molecular index (UMI). In some embodiments, 5′ tail comprises a restriction enzyme recognition sequence. In some embodiments, individual reverse transcription primers comprise at least a portion of 3′ region having a homopolymer sequence, for example poly-A, poly-T, poly-C, poly-G or poly-U. In some embodiments, the reverse transcription primers can hybridize to any portion of an RNA molecule, including 5′ or 3′ end of the RNA molecule, or an internal portion of the RNA molecule.

In some embodiments, the plurality of reverse transcription primers comprises a first sub-population of target-specific reverse transcription primers that hybridize selectively to the first target RNA (e.g., targeted transcriptomics). In some embodiments, the plurality of reverse transcription primers further comprise a second sub-population of target-specific reverse transcription primers that hybridize selectively to the second target RNA. In some embodiments, the target-specific reverse transcription primers comprise a pre-determined sequence at 3′ region which hybridizes to a target RNA molecule. In some embodiments, the pre-determined sequence portion of the reverse transcription primers can be 4-20 bases, or 20-40 bases, or 40-50 bases in length.

In some embodiments, the first sub-population of target-specific reverse transcription primers can selectively hybridize to an RNA transcribed in the cellular sample by a housekeeping gene. In some embodiments, selection of the housekeeping gene may be dependent upon the type of cellular sample to be used for the in situ methods described herein. Exemplary housekeeping genes include glyceraldehyde-3-phosphate dehydrogenase (GAPDH), beta-actins (ACTB), tubulins, PPIA (peptidyl-prolyl cis-trans isomerase), NME4 (NME/NM23 nucleoside diphosphate kinase 4), SMARCALI (SWI/SNF related matrix associated actin dependent regulator of chromatin, subfamily A like 1), and POMK (protein-O-mannose kinase). The skilled artisan can design the first sub-population of target-specific reverse transcription primers to hybridize to RNA transcripts from any of the numerous housekeeping genes.

In some embodiments, the second sub-population of target-specific reverse transcription primers can selectively hybridize to an RNA transcribed from a gene that is expressed in the cellular sample being examined (e.g., a cell-specific or tissue-specific RNA).

In some embodiments, the plurality of reverse transcription primers comprises a first sub-population of random-sequence reverse transcription primers that hybridize to the first target RNA (e.g., whole transcriptomics). In some embodiments, the plurality of reverse transcription primers further comprises a second sub-population of random-sequence reverse transcription primers that hybridize to the second target RNA. In some embodiments, the reverse transcription primers comprise a random and/or degenerate sequence at 3′ region which hybridizes to an RNA molecule. In some embodiments, the random-sequence or the degenerate-sequence portion of the reverse transcription primers can be 4-20 bases, or 20-40 bases, or 40-50 bases in length.

### Sequencing Polymerases

In any of the methods described herein, sequencing polymerases can be used for conducting sequencing reactions. In some embodiments, the sequencing polymerase(s) is/are capable of binding and incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

### Sequencing-by-Binding

In any of the methods described herein, the sequencing comprises conducting sequencing-by-binding (SBB) reactions inside the cellular sample, where the cDNA amplicons are the concatemer molecules. In some embodiments, the sequencing-by-binding (SBB) procedure employs non-labeled chain-terminating nucleotides. In some embodiments, a cycle of sequencing-by-binding (SBB) comprises the steps of (a) sequentially contacting a primed concatemer (e.g., a concatemer annealed to a plurality of sequencing primers) with at least two separate mixtures under ternary complex stabilizing conditions, wherein the at least two separate mixtures each include a polymerase and a nucleotide, whereby the sequentially contacting results in the primed concatemer being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed concatemer, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed concatemer after step (b), thereby producing an extended primer; and (e) repeating steps (a) through (d) at least once on the primed concatemer that comprises the extended primer. Exemplary sequencing-by-binding methods are described in U.S. Pat. Nos. 10,246,744 and 10,731,141 (where the contents of both patents are hereby incorporated by reference in their entireties).

### Nucleotides and Chain-Terminating Nucleotides

In any of the methods described herein, any of the sequencing methods described herein can employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar hydroxyl position where the sugar comprises a ribose or deoxyribose sugar moiety. In some embodiments, the chain terminating moiety is removable/cleavable from 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine) palladium (0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the methods for sequencing described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl) phosphine (TCEP) or bis-sulfo triphenyl phosphine (BS-TPP) or Tri (hydroxyproyl)phosphine (THPP). In some embodiments, the cleaving agent comprises 4-dimethylaminopyridine (4-DMAP).

In some embodiments, in any of the methods for sequencing described herein, the nucleotide comprises a chain terminating moiety which is selected from a group consisting of 3′-deoxy nucleotides, 2′, 3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, in any of the methods for sequencing described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine) palladium (0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl) phosphine (TCEP) or bis-sulfo triphenyl phosphine (BS-TPP) or Tri (hydroxyproyl)phosphine (THPP). In some embodiments, the cleaving agent comprises 4-dimethylaminopyridine (4-DMAP).

In some embodiments, in any of the methods for sequencing described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

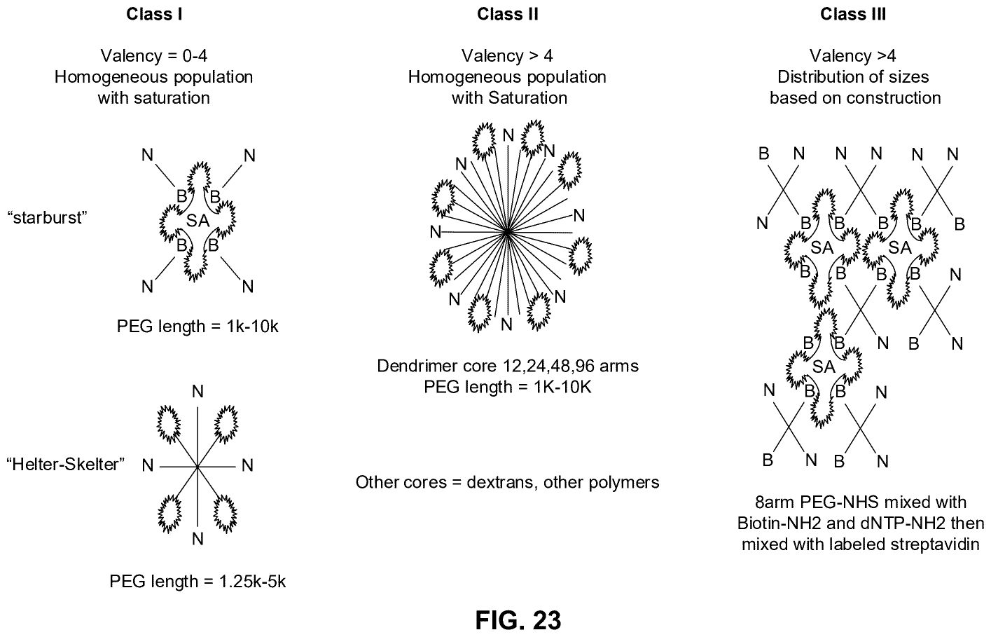

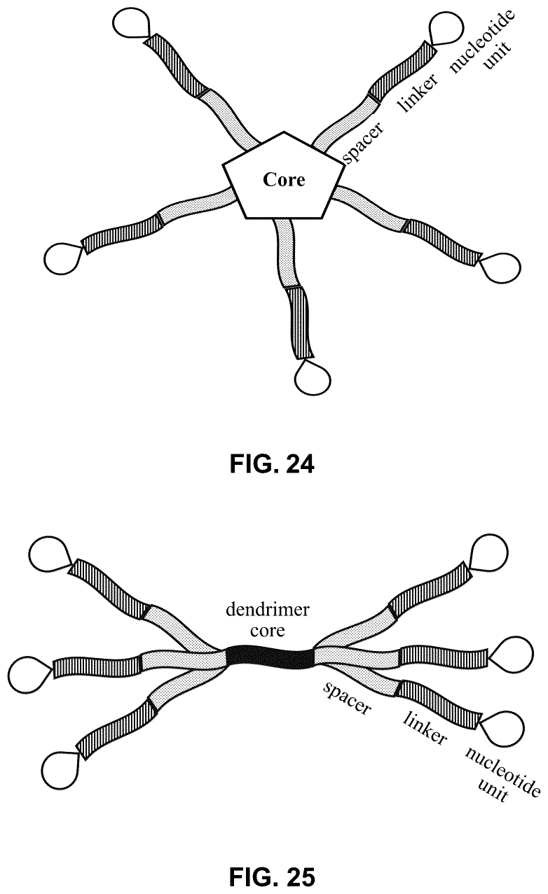

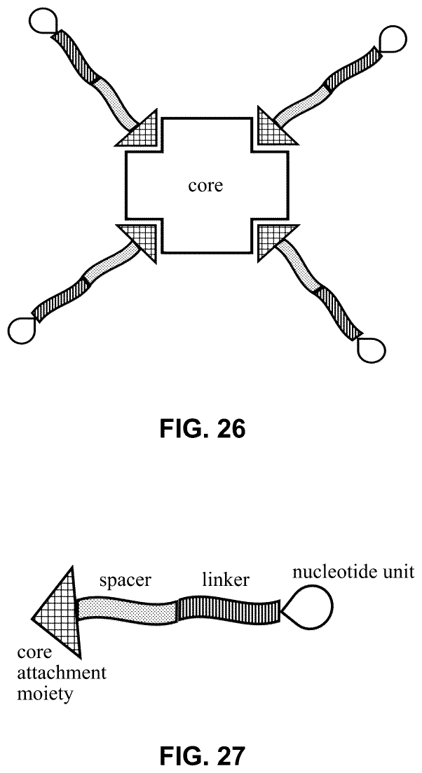

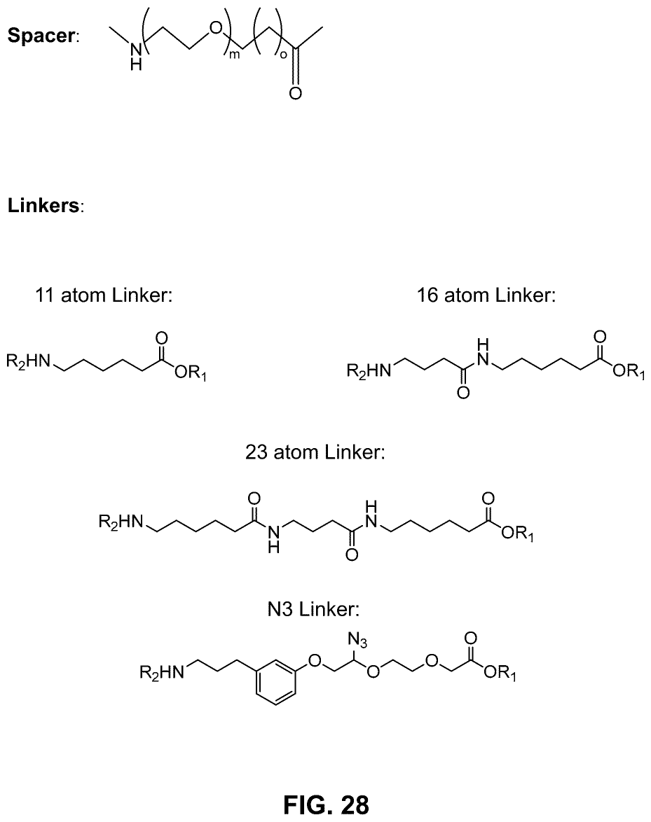

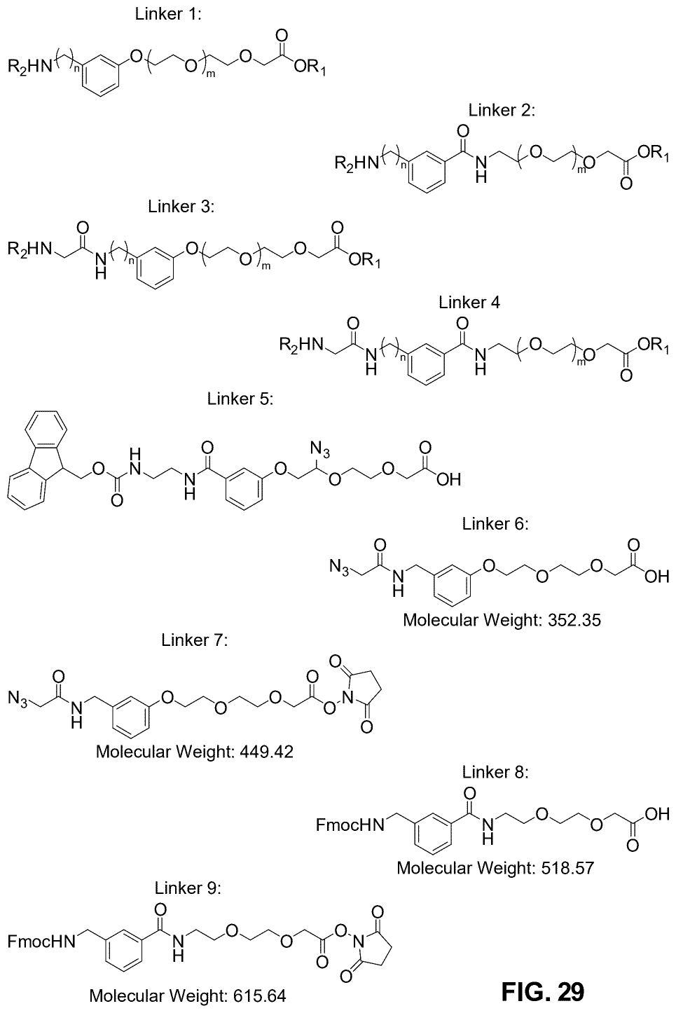

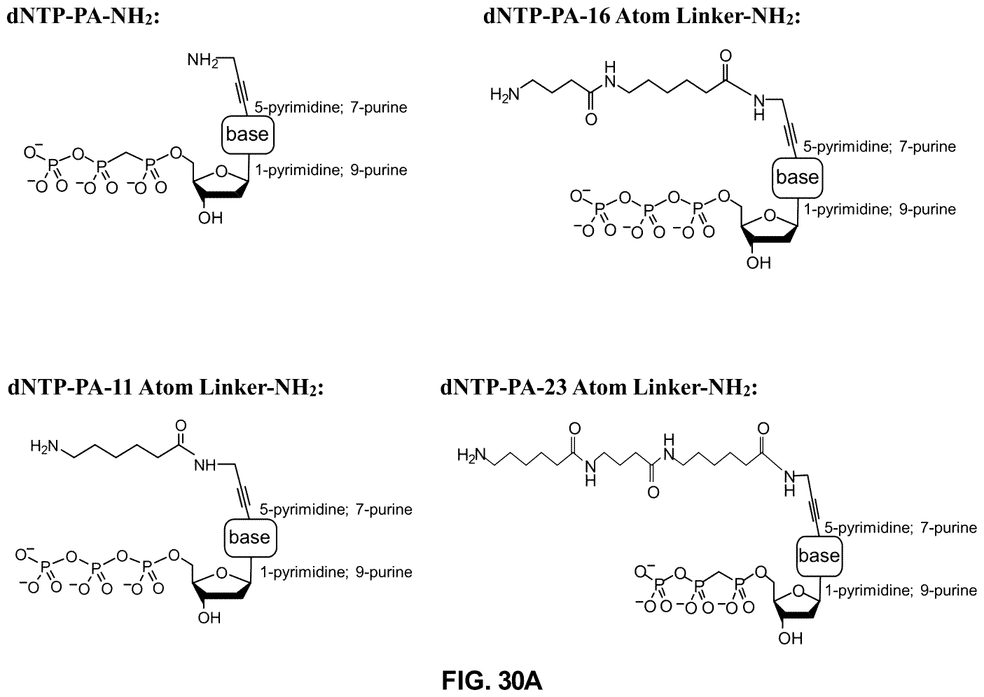

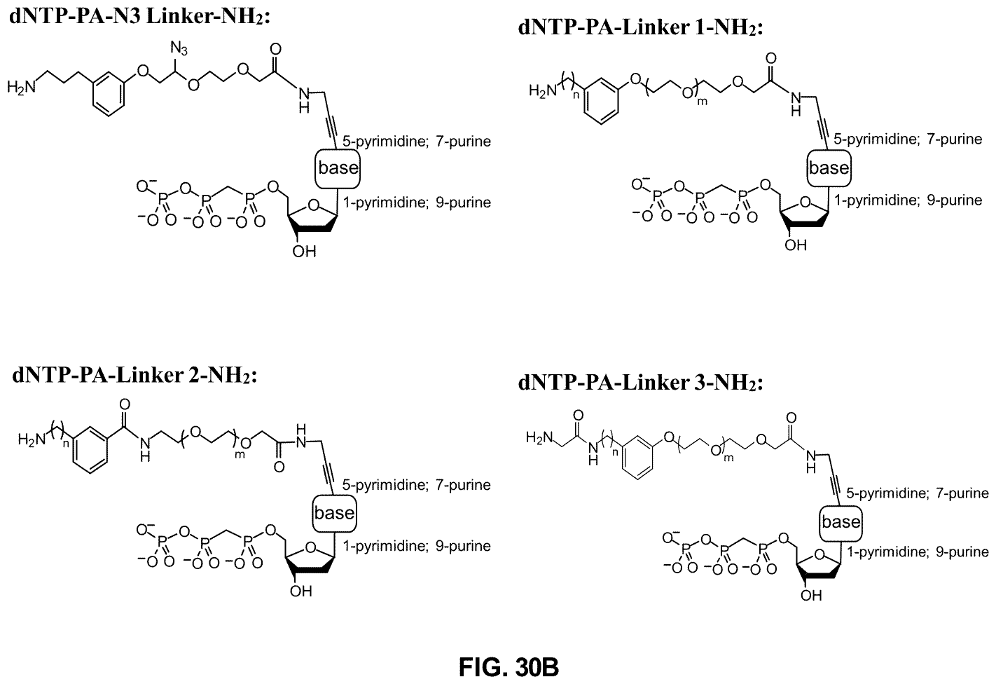

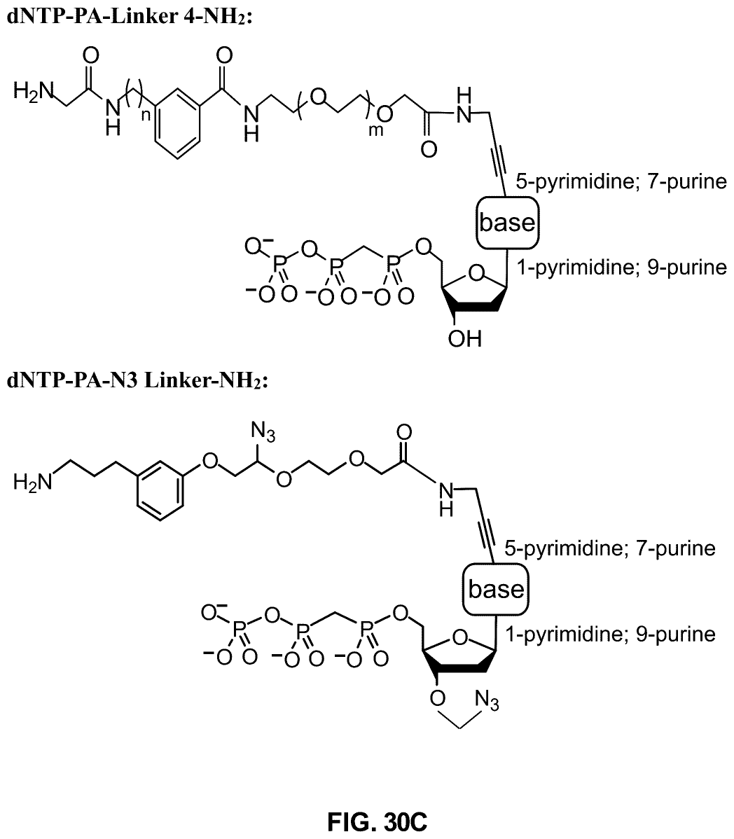

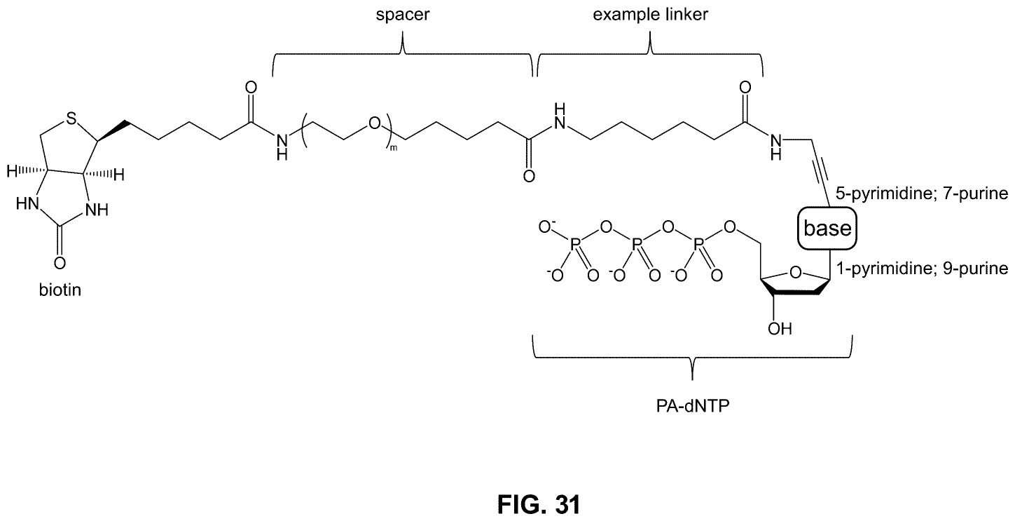

In any of the methods described herein, the sequencing employs at least one multivalent molecule which comprises a plurality of nucleotide arms attached to a core and having any configuration including a starburst, helter skelter, or bottle brush configuration (e.g., FIG. 23). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 subunits. In some embodiments, the linker also includes an aromatic moiety. An exemplary nucleotide arm is shown in FIG. 27. Exemplary multivalent molecules are shown in FIGS. 23-26. An exemplary spacer is shown in FIG. 28 (top) and exemplary linkers are shown in FIG. 28 (bottom) and FIG. 29. Exemplary nucleotides attached to a linker are shown in FIGS. 30A-30C. An exemplary biotinylated nucleotide arm is shown in FIG. 31.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type multivalent molecule having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar hydroxyl position where the sugar comprises a ribose or deoxyribose sugar moiety. In some embodiments, the chain terminating moiety is removable/cleavable from 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine) palladium (0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl) phosphine (TCEP) or bis-sulfo triphenyl phosphine (BS-TPP) or Tri (hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′, 3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises an streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some embodiments, any of the methods for sequencing nucleic acid molecules described herein can include forming a binding complex, where the binding complex comprises (i) a polymerase, a nucleic acid concatemer molecule duplexed with a primer, and a nucleotide, or the binding complex comprises (ii) a polymerase, a nucleic acid concatemer molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

In some embodiments, in any of the sequencing methods that employ multivalent molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. The concatemer template molecule comprises tandem repeat sequences of a sequence of interest and at least one universal sequencing primer binding site. The first and second nucleic acid primers can bind to a sequencing primer binding site along the concatemer template molecule. Exemplary multivalent molecules are shown in FIGS. 23-26.

In some embodiments, in any of the sequencing methods that employ multivalent molecules, the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers can bind to a sequencing primer binding site along the concatemer template molecule. Exemplary multivalent molecules are shown in FIGS. 23-26.

### Flow Cells

In any of the methods described herein, the cellular sample can be deposited onto a solid support (e.g., a flowcell). In some embodiments, the cellular sample is deposited onto a flowcell having walls (e.g., top or first wall, and bottom or second wall) and a gap in-between, where the gap can be filled with a fluid, where the flowcell is positioned in a fluorescence optical imaging system. The cellular sample has a thickness that may require using the imaging system to focus separately on the first and second surfaces of the flowcell, when using a traditional imaging system. For improved imaging of the sequencing reaction of the concatemers in the cellular sample, the flowcell can be positioned in a high performance fluorescence imaging system, which comprises two or more tube lenses which are designed to provide optimal imaging performance for the first and second surfaces of the flowcell at two or more fluorescence wavelengths. In some embodiments, the high-performance imaging system further comprises a focusing mechanism configured to refocus the optical system between acquiring images of the first and second surfaces of the flowcell. In some embodiments, the high performance imaging system is configured to image two or more fields-of-view on at least one of the first flowcell surface or the second flowcell surface.

### Supports and Coatings

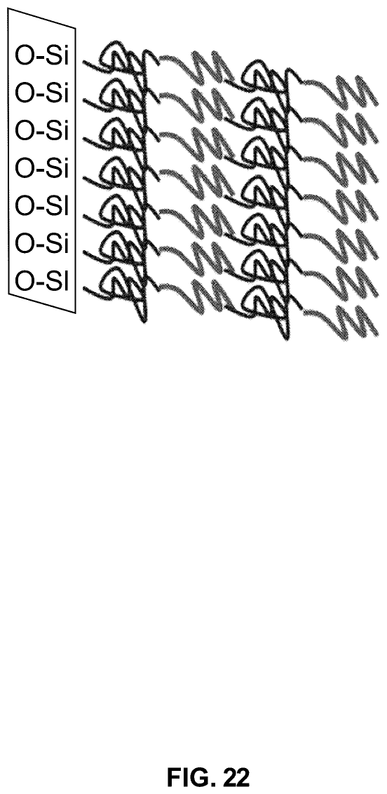

In any of the methods described herein, the solid support comprises a flowcell having a coating that promotes cell adhesion. In some embodiments, the flowcell comprises a support which can be a planar or non-planar support. The support can be solid or semi-solid. In some embodiments, the support can be porous, semi-porous or non-porous. The support can be made of any material such as glass, plastic or a polymer material. In some embodiments, the surface of the support can be coated with one or more compounds to produce a passivated layer on the support (FIG. 22). In some embodiments, the passivated layer forms a porous or semi-porous layer. In some embodiments, the support is coated with a lysine compound, poly-lysine compound, arginine compound or an amino-terminated compound. The support can be coated with an unbranched compound, a branched compound, or a mixture of unbranched and branched compounds. In some embodiments, the support is coated with surface primers for capturing nucleic acids from the cellular sample. Alternatively, the support lacks surface primers.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” can mean within one or more than one standard deviation per the practice in the art. Alternatively, “about” or “approximately” can mean a range of up to 10% (i.e., +10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges can include the endpoints of the ranges and/or subranges.

The term “polony” used herein refers to a nucleic acid library molecule can be clonally amplified in-solution or on-support to generate an amplicon that can serve as a template molecule for sequencing. In some embodiments, a linear library molecule can be circularized to generate a circularized library molecule, and the circularized library molecule can be clonally amplified in-solution or on-support to generate a concatemer. In some embodiments, the concatemer can serve as a nucleic acid template molecule which can be sequenced. The concatemer is sometimes referred to as a polony. In some embodiments, a polony includes nucleotide strands. Although “polony” is used embodiments herein for describing the application of the methods disclosed herein. Such methods may also be useful in other applications that works with clusters that may be generated using various sequencing reactions in NGS.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “polymerase” and its variants, as used herein, comprises any enzyme that can catalyze polymerization of nucleotides (including analogs thereof) into a nucleic acid strand. Typically but not necessarily such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and 3′-5′ exonuclease activity of a DNA polymerase.

As used herein, the term “binding complex” refers to a complex formed by binding together a nucleic acid duplex, a polymerase, and a free nucleotide or a nucleotide unit of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule.

A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

The term “persistence time” and related terms refers to the length of time that a binding complex remains stable without dissociation of any of the components, where the components of the binding complex include a nucleic acid template and nucleic acid primer, a polymerase, a nucleotide unit of a multivalent molecule or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The term “primer” and related terms used herein refers to an oligonucleotide, either natural or synthetic, that is capable of hybridizing with a DNA and/or RNA polynucleotide template to form a duplex molecule. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-mediated primer extension reaction. Alternatively, 3′ end of the primer can lack a 3′ OH moiety, or can include a terminal 3′ blocking group that inhibits nucleotide polymerization in a polymerase-mediated reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The template nucleic acid can be single-stranded or double-stranded, or the template nucleic acid can have single-stranded or double-stranded portions. The sequence of the template nucleic acid can be partially or wholly complementary to the sequence of the complementary strand. The template nucleic acid can be obtained from a naturally-occurring source, recombinant form, or chemically synthesized to include any type of nucleic acid analog. The template nucleic acid can be linear, circular, or other forms. The template nucleic acids can include an insert region having an insert sequence which is also known as a sequence of interest. The template nucleic acids can also include at least one adaptor sequence. The template nucleic acid can be a concatemer having two or tandem copies of a sequence of interest and at least one adaptor sequence. The insert region can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The insert region can be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid can be subjected to nucleic acid analysis, including sequencing and composition analysis.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary.

Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7 mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100:4319-48), acyclic moieties (Martinez, et al., 1999 Nucleic Acids Research 27:1271-1274; Martinez, et al., 1997 Bioorganic & Medicinal Chemistry Letters vol. 7:3013-3016), and other sugar moieties (Joeng, et al., 1993 J. Med. Chem. 36:2627-2638; Kim, et al., 1993 J. Med. Chem. 36:30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′, 3′-dideoxyribosyl; 2′, 3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Ha^{nd}book, (Eugene, Oreg.) 6th Edition; Lakowicz, Principles of Flu^{or}escence Spectroscopy, 2nd Ed., Plenum Press New York (1999), or Hermanson, B^{io}conjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6Iohexyl)-2-((1E,3E)-5-((E)-1-(6-((2,5-dioxopyrrolidin-1-yl)oxy)-6-oxohexyl)-3,3-dimethyl-5-indolin-2-ylidene) penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene) penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene) hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene) hepta-1,3,5-trien-1-yl]-3H-indolium-5-sulfon′te), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure can include but are not limited to: nucleotide transient-binding; nucleotide incorporation; de-blocking; washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

The term “adaptor” and related terms refers to oligonucleotides that can be operably linked (appended) to a target polynucleotide, where the adaptor confers a function to the co-joined adaptor-target molecule. Adaptors comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. An adaptor can include a sequence that is complementary to at least a portion of a primer, such as an amplification primer, a sequencing primer, or a capture primer (e.g., soluble or immobilized capture primers). Adaptors can include a random sequence or degenerate sequence. Adaptors can include at least one inosine residue. Adaptors can include at least one phosphorothioate, phosphorothiolate and/or phosphoramidate linkage. Adaptors can include a barcode sequence which can be used to distinguish polynucleotides (e.g., insert sequences) from different sample sources in a multiplex assay. Adaptors can include a unique identification sequence (e.g., unique molecular index, UMI; or a unique molecular tag) that can be used to uniquely identify a nucleic acid molecule to which the adaptor is appended. In some embodiments, a unique identification sequence can be used to increase error correction and accuracy, reduce the rate of false-positive variant calls and/or increase sensitivity of variant detection. Adaptors can include at least one restriction enzyme recognition sequence, including any one or any combination of two or more selected from a group consisting of type I, type II, type III, type IV, type Hs or type IIB.

The term “universal sequence”, “universal adaptor sequences” and related terms refers to a sequence in a nucleic acid molecule that is common among two or more polynucleotide molecules. For example, adaptors having the same universal sequence can be joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or support-immobilized capture primers).

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support is coated with one or more compounds to produce a passivated layer on the support. In some embodiments, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support.

In some embodiments, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

The present disclosure provides a plurality (e.g., two or more) of nucleic acid templates immobilized to a support. In some embodiments, the immobilized plurality of nucleic acid templates have the same sequence or have different sequences. In some embodiments, individual nucleic acid template molecules in the plurality of nucleic acid templates are immobilized to a different site on the support. In some embodiments, two or more individual nucleic acid template molecules in the plurality of nucleic acid templates are immobilized to a site on the support. In some embodiments, the support comprises a plurality of sites arranged in an array. The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support can have nucleic acid template molecules immobilized at a plurality of sites at a surface density of about 10²-10¹⁵ sites per mm², or more, to form a nucleic acid template array. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid templates to form a nucleic acid template array. In some embodiments, the nucleic acid templates that are immobilized at a plurality of pre-determined sites by hybridization to immobilized surface capture primers, or the nucleic acid templates are covalently attached to the surface capture primers. In some embodiments, the nucleic acid templates that are immobilized at a plurality of pre-determined sites, for example immobilized at 10²-10¹⁵ sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of pre-determined sites. In some embodiments, individual immobilized nucleic acid template molecules comprise one copy of a target sequence of interest, or comprise concatemers having two or more tandem copies of a target sequence of interest.

In some embodiments, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) are immobilized with nucleic acid templates to form a support immobilized with nucleic acid templates. In some embodiments, the nucleic acid templates that are immobilized at a plurality of randomly located sites by hybridization to immobilized surface capture primers, or the nucleic acid templates are covalently attached to the surface capture primer. In some embodiments, the nucleic acid templates that are immobilized at a plurality of randomly located sites, for example immobilized at 10²-10¹⁵ sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of randomly located sites. In some embodiments, individual immobilized nucleic acid template molecules comprise one copy of a target sequence of interest, or comprise concatemers having two or more tandem copies of a target sequence of interest.

In some embodiments, with respect to nucleic acid template molecules immobilized to pre-determined or random sites on the support, the plurality of immobilized nucleic acid template molecules on the support are in fluid communication with each other to permit flowing a solution of reagents (e.g., enzymes including polymerases, multivalent molecules, nucleotides, divalent cations and/or buffers and the like) onto the support so that the plurality of immobilized nucleic acid template molecules on the support can be reacted with the reagents in a massively parallel manner. In some embodiments, the fluid communication of the plurality of immobilized nucleic acid template molecules can be used to conduct nucleotide binding assays and/or conduct nucleotide polymerization reactions (e.g., primer extension or sequencing) on the plurality of immobilized nucleic acid template molecules, and to conduct detection and imaging for massively parallel sequencing. In some embodiments, the term “immobilized” and related terms refer to nucleic acid molecules or enzymes (e.g., polymerases) that are attached to the support at pre-determined or random locations, where the nucleic acid molecules or enzymes are attached directly to a support through covalent bond or non-covalent interaction, or the nucleic acid molecules or enzymes are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 900, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group can be either on the side chain of the polymer or directly attaches to a central core or central backbone of the polymer. The branched polymer can have linear backbone with one or more functional groups coming off the backbone for conjugation. The branched polymer can also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

As used herein, the term “clonally amplified” and it variants refers to a nucleic acid template molecule that has been subjected to one or more amplification reactions either in-solution or on-support. In the case of in-solution amplified template molecules, the resulting amplicons are distributed onto the support. Prior to amplification, the template molecule comprises a sequence of interest and at least one universal adaptor sequence. In some embodiments, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

As used herein, the term “sequencing” and its variants comprise obtaining sequence information from a nucleic acid strand, typically by determining the identity of at least some nucleotides (including their nucleobase components) within the nucleic acid template molecule. While in some embodiments, “sequencing” a given region of a nucleic acid molecule includes identifying each and every nucleotide within the region that is sequenced, in some embodiments “sequencing” comprises methods whereby the identity of only some of the nucleotides in the region is determined, while the identity of some nucleotides remains undetermined or incorrectly determined. Any suitable method of sequencing may be used. In an exemplary embodiment, sequencing can include label-free or ion based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, sequencing includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299 (5607): 682-686; Eid, et al., 2009 Science 323 (5910): 133-138; U.S. Pat. Nos. 7,170,050; 7,302,146; and 7,405,281). An example of sequence-by-hybridization includes SOLID sequencing (e.g., from Life Technologies; WO 2006/084132). An example of sequence-by-binding includes Omniome sequencing (e.g., U.S. Pat. No. 10,246,744).

As used herein, the term “strand displacing” refers to the ability of a polymerase to locally separate strands of double-stranded nucleic acids and synthesize a new strand in a template-based manner. Strand displacing polymerases displace a complementary strand from a template strand and catalyze new strand synthesis. Strand displacing polymerases include mesophilic and thermophilic polymerases. Strand displacing polymerases include wild type enzymes, and variants including exonuclease minus mutants, mutant versions, chimeric enzymes and truncated enzymes. Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi from Expedeon), or variant EquiPhi29 DNA polymerase (e.g., from Thermo Fisher Scientific), or chimeric QualiPhi DNA polymerase (e.g., from 4basebio).

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

When used in reference to nucleic acids, the terms “amplify”, “amplifying”, “amplification”, and other related terms include producing multiple copies of an original polynucleotide template molecule, where the copies comprise a sequence that is complementary to the template sequence, or the copies comprise a sequence that is the same as the template sequence. In some embodiments, the copies comprise a sequence that is substantially identical to a template sequence, or is substantially identical to a sequence that is complementary to the template sequence.

The term “support” as used herein refers to a substrate that is designed for deposition of biological molecules or biological samples for assays and/or analyses. Examples of biological molecules to be deposited onto a support include nucleic acids (e.g., DNA, RNA), polypeptides, saccharides, lipids, a single cell or multiple cells. Examples of biological samples include but are not limited to saliva, phlegm, mucus, blood, plasma, serum, urine, stool, sweat, tears and fluids from tissues or organs.

It is to be appreciated that the Detailed Description section, and not any other section, is intended to be used to interpret the claims. Other sections may set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

Additionally, some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

While preferred embodiments of the present invention have been shown and described herein, it will be obvious to those skilled in the art that such embodiments are provided by way of example only. The breadth and scope of this disclosure should not be limited by any of the above-described exemplary embodiments. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A computer-implemented method for base calling in sequencing data analysis, comprising:
obtaining, by a processor, a plurality of flow cell images of a sample from multiple z levels along an axial axis, where each of the plurality of flow cell images is acquired at a corresponding z level along the axial axis;
generating, by the processor, a plurality of processed images of the plurality of flow cell images;
filtering, by the processor, the plurality of flow cell images based on the plurality of processed images thereby generating a plurality of filtered images;
generating, by the processor, a first maximum intensity projection (MIP) image based on the plurality of filtered images; and
performing, by the processor, base callings using the first MIP image.

2. (canceled)

3. The computer-implemented method of claim 1, wherein the plurality of flow cell images is acquired at one or more sequencing cycles different from a reference cycle.

4. (canceled)

5. (canceled)

6. The computer-implemented method of claim 1, wherein the multiple z levels covers at least some of a thickness of the sample along the axial axis.

7. (canceled)

8. (canceled)

9. The computer-implemented method of claim 1, wherein the sample is an in situ sample immobilized on a support of a flow cell.

10-17. (canceled)

18. The computer-implemented method of claim 1, wherein obtaining the plurality of flow cell images of the sample from multiple z levels along the axial axis comprises:
obtaining the plurality of flow cell images of the sample from multiple z levels along the axial axis from a first color channel.

19. (canceled)

20. (canceled)

21. The computer-implemented method of claim 1, wherein performing, by the processor, base callings using the first MIP image comprises:
performing, by the processor, base callings using the first MIP image from a first color channel and one or more MIP images corresponding to one or more color channels different from the first color channel.

22. The computer-implemented method of claim 1, wherein performing, by the processor, base callings using the first MIP image comprises:
performing, by the processor, base callings using the first MIP image from a first color channel and a corresponding MIP image corresponding to each color channel of the sequencing system different from the first color channel.

23. The computer-implemented method of claim 1, wherein generating the plurality of processed images comprises:
selecting a kernel; and
generating the plurality of processed images by convolving the plurality of flow cell images with the selected kernel.

24. The computer-implemented method of claim 1, wherein generating the plurality of processed images further comprises:
selecting a first kernel and a second kernel;
generating first blurred images by convolving the plurality of flow cell images using the first kernel; and
generating second blurred images by convolving the plurality of flow cell images using the second kernel.

25-31. (canceled)

32. The computer-implemented method of claim 1, wherein filtering the plurality of flow cell images based on the plurality of processed images comprises:
subtracting each of the plurality of processed images from a corresponding flow cell image of the plurality of flow cell images, thereby generating the plurality of filtered images.

33. The computer-implemented method of claim 1, wherein filtering the plurality of flow cell images based on the plurality of processed images further comprises:
adding a predetermined offset to the subtracted images, thereby generating the plurality of filtered images.

34. The computer-implemented method of claim 1, wherein generating the first MIP image based on the plurality of filtered images comprises:
computing a maximum intensity for each pixel of the first MIP image among intensities of the plurality of filtered images at the corresponding pixels.

35-42. (canceled)

43. The computer-implemented method of claim 1, wherein performing base callings using the first MIP image comprises:
performing one or more primary analysis steps to adjust image intensities of polonies in the first MIP image or the one or more MIP images; and
making base calls for the polonies based on the adjusted image intensities; wherein the one or more primary analysis steps comprises:
background subtraction;
image sharpening;
intensity offset adjustment;
color correction;
intensity normalization;
phasing and prephasing correction;
image registration;
quality score estimation; or
a combination thereof.

44-48. (canceled)

49. The computer-implemented method of claim 1, wherein the method further comprises:
obtaining, by the processor, a second MIP image based on the plurality of flow cell images; and
performing image registration of the plurality of flow cell images, the plurality of processed images, the plurality of filtered images, or their combinations based on the second MIP image.

50-54. (canceled)

55. The computer-implemented method of claim 49, wherein performing base callings using the first MIP image comprises:
performing base callings based on image intensities of polonies from the first MIP image and location information of the polonies from the second MIP image of the plurality of flow cell images.

56-66. (canceled)

67. The computer-implemented method of claim 1, wherein obtaining the plurality of flow cell images of the sample comprises:
generating, by the sequencing system, the plurality of flow cell images by conducting one or more cycles of sequencing reactions of the sample immobilized on the support, wherein the plurality flow cell images are generated from two or more different z levels along an axial axis from two or more color channels.

68-87. (canceled)

88. A computer-implemented system for base calling in sequencing data analysis, comprising:
one or more hardware processors;
one or more data storage devices storing instructions executable by the one or more hardware processors to cause the one or more hardware processors to perform operations, the operations comprising:
obtaining, by a processor, a plurality of flow cell images of a sample from multiple z levels along an axial axis, where each of the plurality of flow cell images is acquired at a corresponding location along the axial axis;
generating, by the processor, a plurality of processed images of the plurality of flow cell images;
filtering, by the processor, the plurality of flow cell images based on the plurality of processed images thereby generating a plurality of filtered images;
generating, by the processor, a first maximum intensity projection (MIP) image based on the plurality of filtered images; and
performing, by the processor, base callings using the first MIP image.

89-132. (canceled)

133. The computer-implemented system of claim 1, wherein the method further comprises:
registering, the first MIP images, to one or more images of the sample, wherein the one or more images comprises staining of: membranes, nuclei, or their combinations of the sample.

134-181. (canceled)

182. A method for staining cells or tissue, comprising:
selecting one or more primary antibodies, each of the one or more primary antibody binding specifically to a corresponding protein;
selecting one or more secondary antibodies that binds to the one or more primary antibodies;
labeling the one or more secondary antibodies with a fluorescent label; and
generating one or more images of the corresponding proteins, the one or more images contains fluorescent signal generated from the fluorescent label.

183-187. (canceled)

188. A computer-implemented method for base calling in sequencing data analysis, comprising:
obtaining, by a processor, a first plurality of flow cell images of a sample from multiple z levels along an axial axis, wherein each of the first plurality of flow cell images is acquired at a corresponding z level along the axial axis;
generating, by the processor, a first plurality of processed images of the first plurality of flow cell images;
filtering, by the processor, the first plurality of flow cell images based on the first plurality of processed images thereby generating a first plurality of filtered images;
obtaining, by the processor, a 3D polony map of the sample;
extracting, by the processor, image intensity of polonies based on the 3D polony map from:
a second plurality of flow cell images;
a second plurality of processed images;
a second plurality of filtered images; or
their combinations; and
performing, by the processor, base callings based on the extracted image intensity of the polonies.

189-300. (canceled)
